SEQUENCE LISTING (243 sequences)

SEQ ID NO: 1 - protein, 999 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 2 - protein, 505 aa, Artificial Sequence
  Human MerTK ECD
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLII

SEQ ID NO: 3 - protein, 507 aa, Artificial Sequence
  Cyno MerTK ECD
MGLAPLPLPLLLGLFLPALWSRAITEAREEAKPYPLFPGPLPGSLQTDHTSLLSLPHTSGYQPALMFSPTQPGRPYTGNVAIPRVTSAGSKLLPPLAFKHTVGHIILSEHKDVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIHNSTAHSILISWVPGFDGYSPFRNCSVQVKEVDPLSNGSVMIFNTSASPHMYQIKQLQALANYSIGVSCMNEIGWSAVSPWILASTTEGAPSVAPLNVTVFLNESRDNVDIRWMKPLTKRQAGELVGYRISHVWQSAGISKELLEEVGQNNSRAQISVQVHNATCTVRIAAVTKGGVGPFSDPVKIFIPAHGWVDHAPSSTPAPGNADPVLII

SEQ ID NO: 4 - protein, 497 aa, Artificial Sequence
  Murine MerTK ECD
MVLAPLLLGLLLLPALWSGGTAEKWEETELDQLFSGPLPGRLPVNHRPFSAPHSSRDQLPPPQTGRSHPAHTAAPQVTSTASKLLPPVAFNHTIGHIVLSEHKNVKFNCSINIPNTYQETAGISWWKDGKELLGAHHSITQFYPDEEGVSIIALFSIASVQRSDNGSYFCKMKVNNREIVSDPIYVEVQGLPYFIKQPESVNVTRNTAFNLTCQAVGPPEPVNIFWVQNSSRVNEKPERSPSVLTVPGLTETAVFSCEAHNDKGLTVSKGVHINIKVIPSPPTEVHILNSTAHSILVSWVPGFDGYSPLQNCSIQVKEADRLSNGSVMVFNTSASPHLYEIQQLQALANYSIAVSCRNEIGWSAVSPWILASTTEGAPSVAPLNITVFLNESNNILDIRWTKPPIKRQDGELVGYRISHVWESAGTYKELSEEVSQNGSWAQIPVQIHNATCTVRIAAITKGGIGPFSEPVNIIIPEHSKVDYAPSSTPAPGNTDSM

SEQ ID NO: 5 - protein, 118 aa, Artificial Sequence
  MTK-201 Heavy Chain Variable
QVQLRQSGPGLVQPSQSLSITCTVSGFSLSNYGLHWVRQSPGKGLEWLGVIWSGGITDYNAAFISRLSIIKDNSRSQVFFKMNSLQADDTAIYYCARKGHDPYAMDFWGQGTSVTVSS

SEQ ID NO: 6 - protein, 116 aa, Artificial Sequence
  MTK-202 Heavy Chain Variable
EVQLVESGGGLVKPGGSLKLSCAASGFTFSDFGMHWVRQVPEKGLEWVAFISGGSNTIYYTDTVKGRFTISRDNAKNTLFLQMTSLRSEDTAMYYCVRNGNSFAYWGQGTLVTVSA

SEQ ID NO: 7 - protein, 117 aa, Artificial Sequence
  MTK-203 Heavy Chain Variable
QVQLQQPGAELVKPGASVKVSCKASGYTFTNYWMHWVKQRPGQGLEWIGRIHPFDTETNDNQKFKGKATLTVDKSSSTAYMQLSSLTSEDSAVYYCVIPANGGFAYWGQGTLVTVSA

SEQ ID NO: 8 - protein, 116 aa, Artificial Sequence
  MTK-204 Heavy Chain Variable
EVQLVESGGGLVKPGGSLKLSCAASGFTFSDYGMHWVRQAPEKGLEWVAYISSGTSTIYYEDTVKGRFTISRDNAKNTLFLQMTSLRSEDTAMYYCARGGLLFDYWGQGTTLTVSS

SEQ ID NO: 9 - protein, 121 aa, Artificial Sequence
  MTK-206 Heavy Chain Variable
QVQLQQSGAELARPGASVKMSCKASGYTFTSYTMHWVKQRPGQGLEWIGYINPSSGYIKNSQKFKDKATLTADKSSSTAYMQLSSLTSEDSAVYYCARTVYGYYGSSPDYWGQGTTLTVSS

SEQ ID NO: 10 - protein, 121 aa, Artificial Sequence
  MTK-207 Heavy Chain Variable
QVQLKESGPGLVAPSQSLSITCTVSGISLTSYGVSWVRQPPGKGLEWLGIIWGDGSTNYHSALISRLSISKDNSKSQVFLKLNSLQTDDTATYYCAKGRYGNYEDWYFDVWGTGTTVTVSS

SEQ ID NO: 11 - protein, 118 aa, Artificial Sequence
  MTK-208 Heavy Chain Variable
QVQLQQPGAELVVPGASVRLSCKASGYTFINYWMHWVKQRPGQGLEWIGEIDPSDSYANYNQKFKDKSTLSLDKSSSTAYMQLSSLTSEDSAVYYCARCDYYGRLDYWGQGTTLTVSS

SEQ ID NO: 12 - protein, 114 aa, Artificial Sequence
  MTK-209 Heavy Chain Variable
QVQLQQPGAELVKPGASVKVSCKASGYTFTNYWIHWVKQRPGQGLEWIGRIHPFDSEPNYNQKFKGKATLTVDKSSSTAYMHLTSLTSEDSAVYYCALASFAYWGQGTLVTVSA

SEQ ID NO: 13 - protein, 123 aa, Artificial Sequence
  MTK-210 Heavy Chain Variable
QVQLQQSGPELVKPGASVKISCKASDYEFSRSWMNWVKQRPGKGLEWIGRIYPGDGDTNYNGKFKGKATLTADKSSNTAYMQLNSLTSEDSAVYFCARKGGYYYSISNYFDYWGQGTTLTVSS

SEQ ID NO: 14 - protein, 122 aa, Artificial Sequence
  MTK-211 Heavy Chain Variable
QVQLQQSGAELMKPGASVKLSCKATGYTFTGYWIEWVKQRPGHGLEWIGEILPGSGGSNYNEKFKGKATFTADTSSNTAYIQLSSLTTEDSAIYCCARGEPSYFSDAGFAYWGQGTLVTVSA

SEQ ID NO: 15 - protein, 120 aa, Artificial Sequence
  MTK-212 Heavy Chain Variable
QVQLQQSGPELVKPGASVKISCKASGYAFSSSWVNWVKQRPGKGLEWIGRIYPGDGDSNYNGNFKGKATLTADKSSNTAYLQLSSLTSEDSAVYFCARKSHFIYYTLDYWGQGTPVTVSS

SEQ ID NO: 16 - protein, 119 aa, Artificial Sequence
  MTK-213 Heavy Chain Variable
QVQLQQPGAELVKPGASVKMSCKASGYTFTSYWITWVKQRPGQGHEWIGEIYPGIGSTNYNEKFKSKATLTVDTSSRTAYMQLSSLTSEDSAVYFCARGAYTKYYFDYWGQGTTLTVSS

SEQ ID NO: 17 - protein, 114 aa, Artificial Sequence
  MTK-214 Heavy Chain Variable
QVQLQQPGAELVKPGASVKVSCKASGYTFTNYWMHWMKQRPGQGLEWIGRIHPFDSDTNYNEKFTDKATLTVDKSSSTAYMQLSSLTSEDSAVYYCAIGTLIYWGQGTTLTASS

SEQ ID NO: 18 - protein, 118 aa, Artificial Sequence
  MTK-215 Heavy Chain Variable
EVQLVESGGGLVKPGGSLKLSCAASGFIFSDYGMHWVRQAPEKGLEWVAYISSGSSTIYYGDTVKGRFTISRDNAKNTLFLQMTSLRSEDTAMYYCAKGGRYGAMDYWGHGTSVTVSS

SEQ ID NO: 19 - protein, 116 aa, Artificial Sequence
  MTK-216 Heavy Chain Variable
EVQLVESGGGLVKPGGSLKLSCAASGFTFSDYGMHWVRQAPEKGLEWVAFISSGSTTIYYADTVKGRFTISRDNAKNTLILQMTSLRSEDTAIYYCTRGGFHLDYWGQGTTLTVSS

SEQ ID NO: 20 - protein, 122 aa, Artificial Sequence
  MTK-217 Heavy Chain Variable
QVQLQQPGTEVVKPGASVKMSCRASGYTFTSYWITWVKQRPGQGLEWIGDIHPGSGINNYNEKFRSKATLTVDTSSSTVYMELSSLTSEDSAVYYCAKGGYYGSSYYVMDYWGQGTSVTVSS

SEQ ID NO: 21 - protein, 118 aa, Artificial Sequence
  MTK-218 Heavy Chain Variable
QVQLQQSGPELVKPGTSVKISCKASGYSFTSYYIHWVKQRPGQGLDWIGWIFPGNRNTQYNEKFKGKATLTADTSSSTAYMQLSSLTSEDSAVYYCARLGLIYALDYWGQGTSVTVSS

SEQ ID NO: 22 - protein, 119 aa, Artificial Sequence
  MTK-219 Heavy Chain Variable
QVQLQQSGADLMKPGASVKLSCKATGNTFTGYWIEWVKQRPGHGLEWIGEILTGSGSTNYNERFKGKATFTADTSSNTAYMQLSSLTTEDSAIYYCAGSYFTNSWFPYWGQGSLVTVSA

SEQ ID NO: 23 - protein, 116 aa, Artificial Sequence
  MTK-220 Heavy Chain Variable
EVQLVESGGGLVKPGGSLKLSCAASGFTFSDYGMHWVRQAPEKGLEWVAFISGGSGTIYYEDTVKGRFTISRDNAKNTLFLQMTSLRSEDTAMYYCTRGGNYFDYWGQGTTLTVSS

SEQ ID NO: 24 - protein, 123 aa, Artificial Sequence
  MTK-221 Heavy Chain Variable
QVTLKESGPGMLQSSQTLSLTCSFSGFSLSTSGMGVSWIRQPSGKGLEWLAHIYWDDDKRYNTSLQSRLTISKDTSRNQVFLKITSVDTADTATYFCGRLGAYSAFPYYFDYWGQGTTLTVSS

SEQ ID NO: 25 - protein, 119 aa, Artificial Sequence
  MTK-222 Heavy Chain Variable
QVQLQQSGADLMKPGASVKLSCKATGYTFTGYWIEWVKQRPGHGLEWIGEILTGSGSSNYNEKFKGKATFTADSSSNTAFMQLSSLTTEDSAIYYCAGSYYSNSWFPYWGQGSLVTVSA

SEQ ID NO: 26 - protein, 113 aa, Artificial Sequence
  MTK-223 Heavy Chain Variable
DVQLQESGPGLVKPSQSLSLTCSVTGYSITSGYYWNWIRQFPGNKLEWMGYISYDGTNNYNPSLKNRISITRDTSENQFFLKLNSVTTEDTATYFCVRGAGYWGQGTTLTVSS

SEQ ID NO: 27 - protein, 114 aa, Artificial Sequence
  MTK-224 Heavy Chain Variable
QVQLQQPGAELVKPGASVKVSCKASGYTFTSYWMHWVKQRPGQGLEWIGRIHPFDSDTNYNQKFKGKATLTVDKSSSTAYMQLSSLTSEDSAVYYCSIAGFAYWGQGTLVTVSA

SEQ ID NO: 28 - protein, 123 aa, Artificial Sequence
  MTK-225 Heavy Chain Variable
EVQLQQSGPELVKPGASVKIFCKASGYTFTAYNMDWVKQSHGKSLEWIGDINPNNGGTIYNQKFKGKATLTVDKSSSTAYMELRSLTSEDTAVYYCARRRRYGNTYYWYFDVWGTGTTVTVSS

SEQ ID NO: 29 - protein, 118 aa, Artificial Sequence
  MTK-226 Heavy Chain Variable
QVQLQQSGPELVKPGASVKISCKASGYSFTSYYIHWVKQRPGQGLEWIGWIFPGNGNTKYNENFRGKATLTADTSSSTAYMQLSSLTSEDSAVYYCARLGLIYALDYWGQGTSVTVSS

SEQ ID NO: 30 - protein, 124 aa, Artificial Sequence
  MTK-227 Heavy Chain Variable
EVKLVESGGGLVQPGGSLSLSCAASGFTFTDNYMTWVRQPPGKALEWLGFSRNKANGYTTEYSASVKGRFTISRDSSQSILYLQMNALRAEDSGTYYCARFPPHYYAGGYFDVWGTGTTVTVSS

SEQ ID NO: 31 - protein, 118 aa, Artificial Sequence
  MTK-228 Heavy Chain Variable
QDQLQQSGPELVKPGASVKISCKASGYSFTSYYIHWVKQRPGQGLEWVGWIFPGNGNTKYNERFKGKATLTADTSSSTAYMQLSSLTSEDSAVYYCARLGLIYALDYWGQGTSVTVSS

SEQ ID NO: 32 - protein, 124 aa, Artificial Sequence
  MTK-229 Heavy Chain Variable
QVQLQQSGPELVKPGASVKISCKASGYEFSRSWMNWVKQRPGKGLEWIGRIYPGDGDTNYKGKLKGQATLTADKSSSTAYMQLSSLTFEDSAVYFCARGGGYYYDSRYEGFVYWGQGTVVTVSA

SEQ ID NO: 33 - protein, 116 aa, Artificial Sequence
  MTK-230 Heavy Chain Variable
EVQLQQSGPELVKPGASVRIPCKASGYTFSDYNMDWVKQSHGKSLEWIGNINPNIGGTIYSQKFKGKATLTVDKSSSTAYMELRSLTSEDTAVYYCARGDYGFASWGQGTLVTVSA

SEQ ID NO: 34 - protein, 108 aa, Artificial Sequence
  MTK-201 Light Chain Variable
QIVLTQSPPIMSAFLGERVTMTCTASSSVSSSYLHWYRQMPGSSPKLWIYDTSNLASGVPARFSGSGSGTSYSLTISSMEAEDAATYYCHQYHHSPYTFGGGTKLEIK

SEQ ID NO: 35 - protein, 112 aa, Artificial Sequence
  MTK-202 Light Chain Variable
NIMMTQSPSSLAVSAGEKVTMNCKSSQSVLYSSNQKNYLAWYQQKPGQSPKLLIYWASTRESGVPDRFTGSGSGTDFTLTISNIQAEDLAIYYCHQYFFSWTFGGGTKLEIK

SEQ ID NO: 36 - protein, 107 aa, Artificial Sequence
  MTK-203 Light Chain Variable
ETTVTQSPASLSVATGEKVTIRCITSTDIGDDMNWYQQKPGEPPKVLISEGNSLRPGVPSRFSSSGYGTDFVFTIENTLSEDVADYFCLQSDNLPLTFGSGTKLEIK

SEQ ID NO: 37 - protein, 112 aa, Artificial Sequence
  MTK-204 Light Chain Variable
NIMMTQSPSSLAVSAGEKVTMSCKSSQSVLYSSNQKNYLAWYQQKPGQSPKLLIQWASTRESGVPDRFTGSGSETDFTLTIRSVQAEDLAVYYCHQYFSSWTFGGGTKLEIK

SEQ ID NO: 38 - protein, 107 aa, Artificial Sequence
  MTK-206 Light Chain Variable
DIQMTQSPSSLSASLGERVSLTCRASQDIGSNLNWLQQEPDGTIKRLIYATSSLESGVPKRFSGSRSGSDYSLTISSLESEDFVDYYCLQYDSSPWTFGGGTKLEIK

SEQ ID NO: 39 - protein, 107 aa, Artificial Sequence
  MTK-207 Light Chain Variable
DIQMTQSPASLSASVGETVTITCRASGNIHNYLAWYQQKQGKSPQLLVYNAKTLAAGVPSRFSGSGSGTQYSLKINSLQPEDFGSYYCQHFWSIPLTFGAGTKLELK

SEQ ID NO: 40 - protein, 107 aa, Artificial Sequence
  MTK-208 Light Chain Variable
QVVLTQSPAIMSASPGEKVTITCSASSSVSYMLWFQQKPGTSPKLWIYSTSNLASGVPARFSGSGSGTSYSLTISRLEAEDAATYYCQQRSSYPPITFGAGTKLELK

SEQ ID NO: 41 - protein, 107 aa, Artificial Sequence
  MTK-209 Light Chain Variable
ETTVTQSPASLSMAMGEKATIRCITSTDIDDKMNWYQQKPGKPPKLLISAGNTLRPGVPSRFSSSGYGTDFVLTVENMLSEDVADYYCLQGGALPLTFGSGTKLEIK

SEQ ID NO: 42 - protein, 107 aa, Artificial Sequence
  MTK-210 Light Chain Variable
QIVLTQSPAIMSASPGVKVTMTCSASSSVSYMHWYQQKPGSSPRLLIYDTSNLASGVPVRFSGSGSGTSYSLTISRMEAEDAATYYCQQWSSYPPYTFGGGTKLEIK

SEQ ID NO: 43 - protein, 106 aa, Artificial Sequence
  MTK-211 Light Chain Variable
QIVLTQSPAVMSASPGEKVTMTCSASSSITYMHWYQQKPGTSPKRWIYDTSKLASGVPARFSGSGSGTSYFLTISSMEAEDVATYYCHQRSTYPYTFGGGTKLEIM

SEQ ID NO: 44 - protein, 107 aa, Artificial Sequence
  MTK-212 Light Chain Variable
DIVMTQSHKFKSTSVGDRVSIACKASQDVGTSVAWYQQKPGKSPKLLIYWASSRHTGVPDRFTGSGSGTDFTLTISNVQSEDLTDYFCQQYSTYPYTFGGGTKLEIK

SEQ ID NO: 45 - protein, 108 aa, Artificial Sequence
  MTK-213 Light Chain Variable
DIVMTQSHKFMSTSVGDRVSITCKASQDVGDAVAWYQQKPGQTPKLLIYWASTRHTGVPDRFTGSGSGTDFTLTISNVQSEDLADYFCQQYSSYLMYTFGGGTKLEIK

SEQ ID NO: 46 - protein, 107 aa, Artificial Sequence
  MTK-214 Light Chain Variable
DILLTQSPATLSVTPGETVSLSCRASQSIYKKLHWFQQRSHRSPRLLIKFASDSISGIPSRFTGSGSGTDYTLSINSVKPEDEAIYYCLQGYSTPLTFGAGTKLELK

SEQ ID NO: 47 - protein, 107 aa, Artificial Sequence
  MTK-215 Light Chain Variable
DIVLTQSPATLSVTPGDSVSLSCRASQSISNNLHWYQQKSHESPRLLIKNPSQSISGIPSRFGARGSGTDFTLIINSVETEDLGVYFCQQSNSWPFTFGSGTKLEIK

SEQ ID NO: 48 - protein, 112 aa, Artificial Sequence
  MTK-216 Light Chain Variable
SIMMTQSPSSLAVSAGEKVTMGCKSSQSVLYSANQQNYLAWYQQKPGQSLKLLIYWASTRESGVPDRFTGSGSGTDFTLTISSVQAEDLAVYYCHQYFSSWTFGGGTKLEIK

SEQ ID NO: 49 - protein, 107 aa, Artificial Sequence
  MTK-217 Light Chain Variable
DILLTQSPAILSVSPGERVSFSCRASQTIGTSIHWYQQRTNDSPRLLIKYASESISGIPSRFSGSGSGTDFTLSINSVESEDIADYYCQQSNDWPYTFGGGTKLEIK

SEQ ID NO: 50 - protein, 112 aa, Artificial Sequence
  MTK-218 Light Chain Variable
DAVMTQTPLSLPVSLGDQASISCRSSQSLENSNGHTYLHWYLQKPGQSPQLLIYRVSNRFSGVQDRFSGSGSGTDFTLKISRVEAEDLGVYFCLQVTHVPFTFGSGTKLEIK

SEQ ID NO: 51 - protein, 111 aa, Artificial Sequence
  MTK-219 Light Chain Variable
DIVLTQSPASLAVSLGQRATISCRASESVDSYGNSFMHWYQQKPGQPPKLLIYRAYTLESGIPARFSGSGSRTDFTLTINPVEADDVATYYCQQSNEDPRTFGGGTKLEIK

SEQ ID NO: 52 - protein, 112 aa, Artificial Sequence
  MTK-220 Light Chain Variable
NIMLTQSPSSLAVSAGEKVTMTCKSSQSVLYSSNQKNYLAWYQQKPGQSPKLLIYWASTRESGVPDRFTGSGSGTDFTLTISNVQAEDLAVYYCHQYFSSWTFGGGTKLEIK

SEQ ID NO: 53 - protein, 106 aa, Artificial Sequence
  MTK-221 Light Chain Variable
DIQMTQSPSSLSASLGGKVTITCKASQDIHKHIAWYQHKPGKGPRLLIHYTSTLQPGIPSRFSGSGSGRDYSFSISNLEPEDLATYYCLQYDNLWTFGGGTKLEIK

SEQ ID NO: 54 - protein, 111 aa, Artificial Sequence
  MTK-222 Light Chain Variable
DIVLTQSPVSLAVSLGQRATISCRASESVDSYGSSFMHWYQQKPGQPPKLLIYRASSLESGIPARFSGSGSRTDFTLTINPVETDDVATYYCQQSNEDPRTFGGGTKLEIK

SEQ ID NO: 55 - protein, 113 aa, Artificial Sequence
  MTK-223 Light Chain Variable
DVVMTQTPLSLPVSLGDQASISCRSSQSLVHSDGNTYLHWYLQKPGQSPKLLIYKVSNRFSGVPDRFSGSGSGTDFTLKISRVEAEDLGVYFCSQSTHVPTWTFGGGTKLEIK

SEQ ID NO: 56 - protein, 107 aa, Artificial Sequence
  MTK-224 Light Chain Variable
DIVMTQSPATLSVTPGDRVSLSCRASQIISDYLNWYQQKSHESPRLLIKYASQSISGIPSRFSGSGSGSDFTLSINSVEPEDVGVYYCQNGHSFPLTFGAGTKLELK

SEQ ID NO: 57 - protein, 107 aa, Artificial Sequence
  MTK-225 Light Chain Variable
DIQMTQSPASLSVSVGETVTITCRASENIYSSLAWYQQKQGKSPQLLVYAATNLADGVPSRFSGSGSGTQFYLKINGLQSEDFGSYYCQHFWGTSYTFGGGTKLEIK

SEQ ID NO: 58 - protein, 112 aa, Artificial Sequence
  MTK-226 Light Chain Variable
DAVMTQTPLSLPVSLGDQASISCRSSQSLESSNGNTYLHWYLQKPGQSPQLLIYRVSNRFSGVLDRFSGSGSGTDFTLQISRVEAEDLGVYFCLQVTHVPFTFGSGTKLEIK

SEQ ID NO: 59 - protein, 107 aa, Artificial Sequence
  MTK-227 Light Chain Variable
DIQMTQSPASLSASVGETVTITCRASENIYSYLAWYQQKQGKSPQLLVYNAKTLAEGVPSRFSGSGSGTQFSLKINSLQPADFGSYYCQHYYRTPPTFGGGTKLEIK

SEQ ID NO: 60 - protein, 112 aa, Artificial Sequence
  MTK-228 Light Chain Variable
DAVMTQTPLSLPVSLGDQASISCRSSQSLENSNGNTYLHWYLQKPGQSPQLLIYRVSNRFSGVLDRFSGSGSGTDFTLKISRVEAEDLGVYFCLQVTHVPFTFGSGTKLEIK

SEQ ID NO: 61 - protein, 107 aa, Artificial Sequence
  MTK-229 Light Chain Variable
QIVLTQSPAIMSASPGEEVTMTCSASSSVSYMHWYQQKSGTSPKRWIYDTSKLASGVSGRFSGSGSGTSYSLTISSMEAEDAATYYCQQWSSNPPITFGAGTKLELK

SEQ ID NO: 62 - protein, 111 aa, Artificial Sequence
  MTK-230 Light Chain Variable
DIVLTQSPASLTVSLGQRATISCRASRSVSTSGYSYMHWYQQKPGQPPKLLIYLASNLESGVPARFSGSRSGTDFTLNIHPVEEEDAATYYCQHSREYPFTFGSGTKLEIK

SEQ ID NO: 63 - protein, 5 aa, Artificial Sequence
  HVR-H1 MTK-201
NYGLH

SEQ ID NO: 64 - protein, 5 aa, Artificial Sequence
  HVR-H1 MTK-202
DFGMH

SEQ ID NO: 65 - protein, 5 aa, Artificial Sequence
  HVR-H1 MTK-203,  MTK-208, MTK-214
NYWMH

SEQ ID NO: 66 - protein, 5 aa, Artificial Sequence
  HVR-H1 MTK-204, MTK-215, MTK-216, MTK-220
DYGMH

SEQ ID NO: 67 - protein, 5 aa, Artificial Sequence
  HVR-H1 MTK-206
SYTMH

SEQ ID NO: 68 - protein, 5 aa, Artificial Sequence
  HVR-H1 MTK-207
SYGVS

SEQ ID NO: 69 - protein, 5 aa, Artificial Sequence
  HVR-H1 MTK-209
NYWIH

SEQ ID NO: 70 - protein, 5 aa, Artificial Sequence
  HVR-H1 MTK-210, MTK-229
RSWMN

SEQ ID NO: 71 - protein, 5 aa, Artificial Sequence
  HVR-H1 MTK-211, MTK-219, MTK-222
GYWIE

SEQ ID NO: 72 - protein, 5 aa, Artificial Sequence
  HVR-H1 MTK-212
SSWVN

SEQ ID NO: 73 - protein, 5 aa, Artificial Sequence
  HVR-H1 MTK-213, MTK-217
SYWIT

SEQ ID NO: 74 - protein, 5 aa, Artificial Sequence
  HVR-H1 MTK-218, MTK-226, MTK-228
SYYIH

SEQ ID NO: 75 - protein, 7 aa, Artificial Sequence
  HVR-H1 MTK-221
TSGMGVS

SEQ ID NO: 76 - protein, 6 aa, Artificial Sequence
  HVR-H1 MTK-223
SGYYWN

SEQ ID NO: 77 - protein, 5 aa, Artificial Sequence
  HVR-H1 MTK-224
SYWMH

SEQ ID NO: 78 - protein, 5 aa, Artificial Sequence
  HVR-H1 MTK-225
AYNMD

SEQ ID NO: 79 - protein, 5 aa, Artificial Sequence
  HVR-H1 MTK-227
DNYMT

SEQ ID NO: 80 - protein, 5 aa, Artificial Sequence
  HVR-H1 MTK-230
DYNMD

SEQ ID NO: 81 - protein, 16 aa, Artificial Sequence
  HVR-H2 MTK-201
VIWSGGITDYNAAFIS

SEQ ID NO: 82 - protein, 17 aa, Artificial Sequence
  HVR-H2 MTK-202
FISGGSNTIYYTDTVKG

SEQ ID NO: 83 - protein, 17 aa, Artificial Sequence
  HVR-H2 MTK-203
RIHPFDTETNDNQKFKG

SEQ ID NO: 84 - protein, 17 aa, Artificial Sequence
  HVR-H2 MTK-204
YISSGTSTIYYEDTVKG

SEQ ID NO: 85 - protein, 17 aa, Artificial Sequence
  HVR-H2 MTK-206
YINPSSGYIKNSQKFKD

SEQ ID NO: 86 - protein, 16 aa, Artificial Sequence
  HVR-H2 MTK-207
IIWGDGSTNYHSALIS

SEQ ID NO: 87 - protein, 17 aa, Artificial Sequence
  HVR-H2 MTK-208
EIDPSDSYANYNQKFKD

SEQ ID NO: 88 - protein, 17 aa, Artificial Sequence
  HVR-H2 MTK-209
RIHPFDSEPNYNQKFKG

SEQ ID NO: 89 - protein, 17 aa, Artificial Sequence
  HVR-H2 MTK-210
RIYPGDGDTNYNGKFKG

SEQ ID NO: 90 - protein, 17 aa, Artificial Sequence
  HVR-H2 MTK-211
EILPGSGGSNYNEKFKG

SEQ ID NO: 91 - protein, 17 aa, Artificial Sequence
  HVR-H2 MTK-212
RIYPGDGDSNYNGNFKG

SEQ ID NO: 92 - protein, 17 aa, Artificial Sequence
  HVR-H2 MTK-213
EIYPGIGSTNYNEKFKS

SEQ ID NO: 93 - protein, 17 aa, Artificial Sequence
  HVR-H2 MTK-214
RIHPFDSDTNYNEKFTD

SEQ ID NO: 94 - protein, 17 aa, Artificial Sequence
  HVR-H2 MTK-215
YISSGSSTIYYGDTVKG

SEQ ID NO: 95 - protein, 17 aa, Artificial Sequence
  HVR-H2 MTK-216
FISSGSTTIYYADTVKG

SEQ ID NO: 96 - protein, 17 aa, Artificial Sequence
  HVR-H2 MTK-217
DIHPGSGINNYNEKFRS

SEQ ID NO: 97 - protein, 17 aa, Artificial Sequence
  HVR-H2 MTK-218
WIFPGNRNTQYNEKFKG

SEQ ID NO: 98 - protein, 17 aa, Artificial Sequence
  HVR-H2 MTK-219
EILTGSGSTNYNERFKG

SEQ ID NO: 99 - protein, 17 aa, Artificial Sequence
  HVR-H2 MTK-220
FISGGSGTIYYEDTVKG

SEQ ID NO: 100 - protein, 16 aa, Artificial Sequence
  HVR-H2 MTK-221
HIYWDDDKRYNTSLQS

SEQ ID NO: 101 - protein, 17 aa, Artificial Sequence
  HVR-H2 MTK-222
EILTGSGSSNYNEKFKG

SEQ ID NO: 102 - protein, 16 aa, Artificial Sequence
  HVR-H2 MTK-223
YISYDGTNNYNPSLKN

SEQ ID NO: 103 - protein, 17 aa, Artificial Sequence
  HVR-H2 MTK-224
RIHPFDSDTNYNQKFKG

SEQ ID NO: 104 - protein, 17 aa, Artificial Sequence
  HVR-H2 MTK-225
DINPNNGGTIYNQKFKG

SEQ ID NO: 105 - protein, 17 aa, Artificial Sequence
  HVR-H2 MTK-226
WIFPGNGNTKYNENFRG

SEQ ID NO: 106 - protein, 19 aa, Artificial Sequence
  HVR-H2 MTK-227
FSRNKANGYTTEYSASVKG

SEQ ID NO: 107 - protein, 17 aa, Artificial Sequence
  HVR-H2 MTK-228
WIFPGNGNTKYNERFKG

SEQ ID NO: 108 - protein, 17 aa, Artificial Sequence
  HVR-H2 MTK-229
RIYPGDGDTNYKGKLKG

SEQ ID NO: 109 - protein, 17 aa, Artificial Sequence
  HVR-H2 MTK-230
NINPNIGGTIYSQKFKG

SEQ ID NO: 110 - protein, 10 aa, Artificial Sequence
  HVR-H3 MTK-201
KGHDPYAMDF

SEQ ID NO: 111 - protein, 7 aa, Artificial Sequence
  HVR-H3 MTK-202
NGNSFAY

SEQ ID NO: 112 - protein, 8 aa, Artificial Sequence
  HVR-H3 MTK-203
PANGGFAY

SEQ ID NO: 113 - protein, 7 aa, Artificial Sequence
  HVR-H3 MTK-204
GGLLFDY

SEQ ID NO: 114 - protein, 12 aa, Artificial Sequence
  HVR-H3 MTK-206
TVYGYYGSSPDY

SEQ ID NO: 115 - protein, 13 aa, Artificial Sequence
  HVR-H3 MTK-207
GRYGNYEDWYFDV

SEQ ID NO: 116 - protein, 9 aa, Artificial Sequence
  HVR-H3 MTK-208
CDYYGRLDY

SEQ ID NO: 117 - protein, 5 aa, Artificial Sequence
  HVR-H3 MTK-209
ASFAY

SEQ ID NO: 118 - protein, 14 aa, Artificial Sequence
  HVR-H3 MTK-210
KGGYYYSISNYFDY

SEQ ID NO: 119 - protein, 13 aa, Artificial Sequence
  HVR-H3 MTK-211
GEPSYFSDAGFAY

SEQ ID NO: 120 - protein, 11 aa, Artificial Sequence
  HVR-H3 MTK-212
KSHFIYYTLDY

SEQ ID NO: 121 - protein, 10 aa, Artificial Sequence
  HVR-H3 MTK-213
GAYTKYYFDY

SEQ ID NO: 122 - protein, 5 aa, Artificial Sequence
  HVR-H3 MTK-214
GTLIY

SEQ ID NO: 123 - protein, 9 aa, Artificial Sequence
  HVR-H3 MTK-215
GGRYGAMDY

SEQ ID NO: 124 - protein, 7 aa, Artificial Sequence
  HVR-H3 MTK-216
GGFHLDY

SEQ ID NO: 125 - protein, 13 aa, Artificial Sequence
  HVR-H3 MTK-217
GGYYGSSYYVMDY

SEQ ID NO: 126 - protein, 9 aa, Artificial Sequence
  HVR-H3 MTK-218, MTK-226, MTK-228
LGLIYALDY

SEQ ID NO: 127 - protein, 10 aa, Artificial Sequence
  HVR-H3 MTK-219
SYFTNSWFPY

SEQ ID NO: 128 - protein, 7 aa, Artificial Sequence
  HVR-H3 MTK-220
GGNYFDY

SEQ ID NO: 129 - protein, 13 aa, Artificial Sequence
  HVR-H3 MTK-221
LGAYSAFPYYFDY

SEQ ID NO: 130 - protein, 10 aa, Artificial Sequence
  HVR-H3 MTK-222
SYYSNSWFPY

SEQ ID NO: 131 - protein, 4 aa, Artificial Sequence
  HVR-H3 MTK-223
GAGY

SEQ ID NO: 132 - protein, 5 aa, Artificial Sequence
  HVR-H3 MTK-224
AGFAY

SEQ ID NO: 133 - protein, 14 aa, Artificial Sequence
  HVR-H3 MTK-225
RRRYGNTYYWYFDV

SEQ ID NO: 134 - protein, 13 aa, Artificial Sequence
  HVR-H3 MTK-227
FPPHYYAGGYFDV

SEQ ID NO: 135 - protein, 15 aa, Artificial Sequence
  HVR-H3 MTK-229
GGGYYYDSRYEGFVY

SEQ ID NO: 136 - protein, 7 aa, Artificial Sequence
  HVR-H3 MTK-230
GDYGFAS

SEQ ID NO: 137 - protein, 12 aa, Artificial Sequence
  HVR-L1 MTK-201
TASSSVSSSYLH

SEQ ID NO: 138 - protein, 17 aa, Artificial Sequence
  HVR-L1 MTK-202, MTK-204, MTK-220
KSSQSVLYSSNQKNYLA

SEQ ID NO: 139 - protein, 11 aa, Artificial Sequence
  HVR-L1 MTK-203
ITSTDIGDDMN

SEQ ID NO: 140 - protein, 11 aa, Artificial Sequence
  HVR-L1 MTK-206
RASQDIGSNLN

SEQ ID NO: 141 - protein, 11 aa, Artificial Sequence
  HVR-L1 MTK-207
RASGNIHNYLA

SEQ ID NO: 142 - protein, 10 aa, Artificial Sequence
  HVR-L1 MTK-208
SASSSVSYML

SEQ ID NO: 143 - protein, 11 aa, Artificial Sequence
  HVR-L1 MTK-209
ITSTDIDDKMN

SEQ ID NO: 144 - protein, 10 aa, Artificial Sequence
  HVR-L1 MTK-210, MTK-229
SASSSVSYMH

SEQ ID NO: 145 - protein, 10 aa, Artificial Sequence
  HVR-L1 MTK-211
SASSSITYMH

SEQ ID NO: 146 - protein, 11 aa, Artificial Sequence
  HVR-L1 MTK-205, MTK-212
KASQDVGTSVA

SEQ ID NO: 147 - protein, 11 aa, Artificial Sequence
  HVR-L1 MTK-213
KASQDVGDAVA

SEQ ID NO: 148 - protein, 11 aa, Artificial Sequence
  HVR-L1 MTK-214
RASQSIYKKLH

SEQ ID NO: 149 - protein, 11 aa, Artificial Sequence
  HVR-L1 MTK-215
RASQSISNNLH

SEQ ID NO: 150 - protein, 17 aa, Artificial Sequence
  HVR-L1 MTK-216
KSSQSVLYSANQQNYLA

SEQ ID NO: 151 - protein, 11 aa, Artificial Sequence
  HVR-L1 MTK-217
RASQTIGTSIH

SEQ ID NO: 152 - protein, 16 aa, Artificial Sequence
  HVR-L1 MTK-218
RSSQSLENSNGHTYLH

SEQ ID NO: 153 - protein, 15 aa, Artificial Sequence
  HVR-L1 MTK-219
RASESVDSYGNSFMH

SEQ ID NO: 154 - protein, 11 aa, Artificial Sequence
  HVR-L1 MTK-221
KASQDIHKHIA

SEQ ID NO: 155 - protein, 15 aa, Artificial Sequence
  HVR-L1 MTK-222
RASESVDSYGSSFMH

SEQ ID NO: 156 - protein, 16 aa, Artificial Sequence
  HVR-L1 MTK-223, MTK-231
RSSQSLVHSDGNTYLH

SEQ ID NO: 157 - protein, 11 aa, Artificial Sequence
  HVR-L1 MTK-224
RASQIISDYLN

SEQ ID NO: 158 - protein, 11 aa, Artificial Sequence
  HVR-L1 MTK-225
RASENIYSSLA

SEQ ID NO: 159 - protein, 16 aa, Artificial Sequence
  HVR-L1 MTK-226
RSSQSLESSNGNTYLH

SEQ ID NO: 160 - protein, 11 aa, Artificial Sequence
  HVR-L1 MTK-227
RASENIYSYLA

SEQ ID NO: 161 - protein, 16 aa, Artificial Sequence
  HVR-L1 MTK-228
RSSQSLENSNGNTYLH

SEQ ID NO: 162 - protein, 15 aa, Artificial Sequence
  HVR-L1 MTK-230
RASRSVSTSGYSYMH

SEQ ID NO: 163 - protein, 7 aa, Artificial Sequence
  HVR-L2 MTK-201, MTK-210
DTSNLAS

SEQ ID NO: 164 - protein, 7 aa, Artificial Sequence
  HVR-L2 MTK-202, MTK-204, MTK-216, MTK-220
WASTRES

SEQ ID NO: 165 - protein, 7 aa, Artificial Sequence
  HVR-L2 MTK-203
EGNSLRP

SEQ ID NO: 166 - protein, 7 aa, Artificial Sequence
  HVR-L2 MTK-206
ATSSLES

SEQ ID NO: 167 - protein, 7 aa, Artificial Sequence
  HVR-L2 MTK-207
NAKTLAA

SEQ ID NO: 168 - protein, 7 aa, Artificial Sequence
  HVR-L2 MTK-208
STSNLAS

SEQ ID NO: 169 - protein, 7 aa, Artificial Sequence
  HVR-L2 MTK-209
AGNTLRP

SEQ ID NO: 170 - protein, 7 aa, Artificial Sequence
  HVR-L2 MTK-211, MTK-229
DTSKLAS

SEQ ID NO: 171 - protein, 7 aa, Artificial Sequence
  HVR-L2 MTK-212
WASSRHT

SEQ ID NO: 172 - protein, 7 aa, Artificial Sequence
  HVR-L2 MTK-213, MTK-232
WASTRHT

SEQ ID NO: 173 - protein, 7 aa, Artificial Sequence
  HVR-L2 MTK-214
FASDSIS

SEQ ID NO: 174 - protein, 7 aa, Artificial Sequence
  HVR-L2 MTK-215
NPSQSIS

SEQ ID NO: 175 - protein, 7 aa, Artificial Sequence
  HVR-L2 MTK-217
YASESIS

SEQ ID NO: 176 - protein, 7 aa, Artificial Sequence
  HVR-L2 MTK-218, MTK-226, MTK-228
RVSNRFS

SEQ ID NO: 177 - protein, 7 aa, Artificial Sequence
  HVR-L2 MTK-219
RAYTLES

SEQ ID NO: 178 - protein, 7 aa, Artificial Sequence
  HVR-L2 MTK-221
YTSTLQP

SEQ ID NO: 179 - protein, 7 aa, Artificial Sequence
  HVR-L2 MTK-222
RASSLES

SEQ ID NO: 180 - protein, 7 aa, Artificial Sequence
  HVR-L2 MTK-223, MTK-231
KVSNRFS

SEQ ID NO: 181 - protein, 7 aa, Artificial Sequence
  HVR-L2 MTK-224
YASQSIS

SEQ ID NO: 182 - protein, 7 aa, Artificial Sequence
  HVR-L2 MTK-225
AATNLAD

SEQ ID NO: 183 - protein, 7 aa, Artificial Sequence
  HVR-L2 MTK-227
NAKTLAE

SEQ ID NO: 184 - protein, 7 aa, Artificial Sequence
  HVR-L2 MTK-230
LASNLES

SEQ ID NO: 185 - protein, 9 aa, Artificial Sequence
  HVR-L3 MTK-201
HQYHHSPYT

SEQ ID NO: 186 - protein, 8 aa, Artificial Sequence
  HVR-L3 MTK-202
HQYFFSWT

SEQ ID NO: 187 - protein, 9 aa, Artificial Sequence
  HVR-L3 MTK-203
LQSDNLPLT

SEQ ID NO: 188 - protein, 8 aa, Artificial Sequence
  HVR-L3 MTK-204, MTK-216, MTK-220
HQYFSSWT

SEQ ID NO: 189 - protein, 9 aa, Artificial Sequence
  HVR-L3 MTK-206
LQYDSSPWT

SEQ ID NO: 190 - protein, 9 aa, Artificial Sequence
  HVR-L3 MTK-207
QHFWSIPLT

SEQ ID NO: 191 - protein, 10 aa, Artificial Sequence
  HVR-L3 MTK-208
QQRSSYPPIT

SEQ ID NO: 192 - protein, 9 aa, Artificial Sequence
  HVR-L3 MTK-209
LQGGALPLT

SEQ ID NO: 193 - protein, 10 aa, Artificial Sequence
  HVR-L3 MTK-210
QQWSSYPPYT

SEQ ID NO: 194 - protein, 9 aa, Artificial Sequence
  HVR-L3 MTK-211
HQRSTYPYT

SEQ ID NO: 195 - protein, 9 aa, Artificial Sequence
  HVR-L3 MTK-212
QQYSTYPYT

SEQ ID NO: 196 - protein, 10 aa, Artificial Sequence
  HVR-L3 MTK-213
QQYSSYLMYT

SEQ ID NO: 197 - protein, 9 aa, Artificial Sequence
  HVR-L3 MTK-214
LQGYSTPLT

SEQ ID NO: 198 - protein, 9 aa, Artificial Sequence
  HVR-L3 MTK-215
QQSNSWPFT

SEQ ID NO: 199 - protein, 9 aa, Artificial Sequence
  HVR-L3 MTK-217
QQSNDWPYT

SEQ ID NO: 200 - protein, 9 aa, Artificial Sequence
  HVR-L3 MTK-218, MTK-226, MTK-228
LQVTHVPFT

SEQ ID NO: 201 - protein, 9 aa, Artificial Sequence
  HVR-L3 MTK-219, MTK-222
QQSNEDPRT

SEQ ID NO: 202 - protein, 8 aa, Artificial Sequence
  HVR-L3 MTK-221
LQYDNLWT

SEQ ID NO: 203 - protein, 10 aa, Artificial Sequence
  HVR-L3 MTK-223
SQSTHVPTWT

SEQ ID NO: 204 - protein, 9 aa, Artificial Sequence
  HVR-L3 MTK-224
QNGHSFPLT

SEQ ID NO: 205 - protein, 9 aa, Artificial Sequence
  HVR-L3 MTK-225
QHFWGTSYT

SEQ ID NO: 206 - protein, 9 aa, Artificial Sequence
  HVR-L3 MTK-227
QHYYRTPPT

SEQ ID NO: 207 - protein, 10 aa, Artificial Sequence
  HVR-L3 MTK-229
QQWSSNPPIT

SEQ ID NO: 208 - protein, 9 aa, Artificial Sequence
  HVR-L3 MTK-230
QHSREYPFT

SEQ ID NO: 209 - protein, 113 aa, Artificial Sequence
  MTK-231 Heavy Chain Variable
DVQLQESGPGLVKPSQSLSLTCSVTGYSITSGYYWNWIRQFPGNKLEWMGYISYDGTYNYHPSLKNRISITRDTSKNQFFLTLNSVTTEDTATFYCTRGGGYWGQGTSVTVSS

SEQ ID NO: 210 - protein, 117 aa, Artificial Sequence
  MTK-232 Heavy Chain Variable
EVQLQESGPSLVKPSQTLSLTCSVTGDSITSGYWNWIRKFPGNKLEYMGYINYSGSTNYNPSLKSRISITRDTSKNQYYLQLNSVTTEDTATYYCARWNYLYYFDYWGQGTTLTVSS

SEQ ID NO: 211 - protein, 112 aa, Artificial Sequence
  MTK-231 Light Chain Variable
DVVMTQTPLSLPVSLGDQASISCRSSQSLVHSDGNTYLHWYLQKPGQSPKLLIYKVSNRFSGVPDRFSGSGSGTDFTLKISRLEAEDLGIYFCSQTTHIPLTFGSGTKLEIK

SEQ ID NO: 212 - protein, 107 aa, Artificial Sequence
  MTK-232 Light Chain Variable
DSVMTQSHKFMSTLVGDRVSITCKASQDVGTAVAWYQQKPGQSPKVLIYWASTRHTGVPDRFTGSGSGTDFTLTITNVQSEDLADYFCHQYSTYPYTFGGGTKLEIK

SEQ ID NO: 213 - protein, 6 aa, Artificial Sequence
  HVR-H1 MTK-231
SGYYWN

SEQ ID NO: 214 - protein, 5 aa, Artificial Sequence
  HVR-H1 MTK-232
SGYWN

SEQ ID NO: 215 - protein, 16 aa, Artificial Sequence
  HVR-H2 MTK-231
YISYDGTYNYHPSLKN

SEQ ID NO: 216 - protein, 16 aa, Artificial Sequence
  HVR-H2 MTK-232
YINYSGSTNYNPSLKS

SEQ ID NO: 217 - protein, 4 aa, Artificial Sequence
  HVR-H3 MTK-231
GGGY

SEQ ID NO: 218 - protein, 9 aa, Artificial Sequence
  HVR-H3 MTK-232
WNYLYYFDY

SEQ ID NO: 219 - protein, 9 aa, Artificial Sequence
  HVR-L3 MTK-231
SQTTHIPLT

SEQ ID NO: 220 - protein, 11 aa, Artificial Sequence
  HVR-L1 MTK-232
KASQDVGTAVA

SEQ ID NO: 221 - protein, 9 aa, Artificial Sequence
  HVR-L3 MTK-232
HQYSTYPYT

SEQ ID NO: 222 - protein, 117 aa, Artificial Sequence
  MTK-205 Heavy Chain Variable
EVQLQQSGPELVKPGASVKISCKTSGYTFTEYIMHWVKQSHGKSLEWIGGINPNNLGIIYNQKFKGKATLTVDKSSSTAYMELRSLTSEDSEVYYCARDGYFGFDYWGQGTTLKVSS

SEQ ID NO: 223 - protein, 107 aa, Artificial Sequence
  MTK-205 Light Chain Variable
DIVMTQSHKFMSTSVGDRVSITCKASQDVGTSVAWYQQKPGHSPKLLIYWTSTRRTGVPDRFTGSGSGTDFTLTISNVQSEDLADYFCHQYNSYPLTFGAGTKLELK

SEQ ID NO: 224 - protein, 5 aa, Artificial Sequence
  HVR-H1 MTK-205
EYIMH

SEQ ID NO: 225 - protein, 17 aa, Artificial Sequence
  HVR-H2 MTK-205
GINPNNLGIIYNQKFKG

SEQ ID NO: 226 - protein, 8 aa, Artificial Sequence
  HVR-H3 MTK-205
DGYFGFDY

SEQ ID NO: 227 - protein, 7 aa, Artificial Sequence
  HVR-L2 MTK-205
WTSTRRT

SEQ ID NO: 228 - protein, 9 aa, Artificial Sequence
  HVR-L3 MTK-205
HQYNSYPLT

SEQ ID NO: 229 - protein, 67 aa, Artificial Sequence
  N-terminal domain (NT) of human MerTK ECD
AITEAREEAKPYPLFPGPFPGSLQTDHTPLLSLPHASGYQPALMFSPTQPGRPHTGNVAIPQVTSVE

SEQ ID NO: 230 - protein, 107 aa, Artificial Sequence
  Immunoglobulin-like domain (Ig1) of human MerTK ECD
SKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQ

SEQ ID NO: 231 - protein, 85 aa, Artificial Sequence
  Immunoglobulin-like domain (Ig2) of human MerTK ECD
GLPHFTKQPESMNVTRNTAFNLTCQAVGPPEPVNIFWVQNSSRVNEQPEKSPSVLTVPGLTEMAVFSCEAHNDKGLTVSKGVQIN

SEQ ID NO: 232 - protein, 99 aa, Artificial Sequence
  Fibronectin type III domain (FN1) of human MerTK ECD
IKAIPSPPTEVSIRNSTAHSILISWVPGFDGYSPFRNCSIQVKEADPLSNGSVMIFNTSALPHLYQIKQLQALANYSIGVSCMNEIGWSAVSPWILAST

SEQ ID NO: 233 - protein, 98 aa, Artificial Sequence
  Fibronectin type III domain (FN2) of human MerTK ECD
TEGAPSVAPLNVTVFLNESSDNVDIRWMKPPTKQQDGELVGYRISHVWQSAGISKELLEEVGQNGSRARISVQVHNATCTVRIAAVTRGGVGPFSDPV

SEQ ID NO: 234 - protein, 29 aa, Artificial Sequence
  Juxta membrane domain region (JM) of human MerTK ECD
KIFIPAHGWVDYAPSSTPAPGNADPVLII

SEQ ID NO: 235 - protein, 27 aa, Artificial Sequence
  N-terminal domain (NT) of human Axl ECD
APRGTQAEESPFVGNPGNITGARGLTG

SEQ ID NO: 236 - protein, 84 aa, Artificial Sequence
  Immunoglobulin-like domain (Ig1) of human Axl ECD
TLRCQLQVQGEPPEVHWLRDGQILELADSTQTQVPLGEDEQDDWIVVSQLRITSLQLSDTGQYQCLVFLGHQTFVSQPGYVGLE

SEQ ID NO: 237 - protein, 89 aa, Artificial Sequence
  Immunoglobulin-like domain (Ig2) of human Axl ECD
GLPYFLEEPEDRTVAANTPFNLSCQAQGPPEPVDLLWLQDAVPLATAPGHGPQRSLHVPGLNKTSSFSCEAHNAKGVTTSRTATITVLP

SEQ ID NO: 238 - protein, 106 aa, Artificial Sequence
  Fibronectin type III domain (FN1) of human Axl ECD
QQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEG

SEQ ID NO: 239 - protein, 87 aa, Artificial Sequence
  Fibronectin type III domain (FN2) of human Axl ECD
VPLGPPENISATRNGSQAFVHWQEPRAPLQGTLLGYRLAYQGQDTPEVLMDIGLRQEVTLELQGDGSVSNLTVCVAAYTAAGDGPWS

SEQ ID NO: 240 - protein, 33 aa, Artificial Sequence
  Juxta membrane domain (JM) of human Axl ECD
LPVPLEAWRPGQAQPVHQLVKEPSTPAFSWPWW

SEQ ID NO: 241 - protein, 19 aa, Artificial Sequence
  Signal sequence in MerTK constructs
MGWSCIILFLVATATGVHS

SEQ ID NO: 242 - protein, 16 aa, Artificial Sequence
  Signal sequence in Axl constructs
MGWSCIILFLVATATG

SEQ ID NO: 243 - protein, 27 aa, Artificial Sequence
  Linker+His+Avi tag
GGSGHHHHHHGGGLNDIFEAQKIEWHE

(19) United States
(12) Patent Application Publication
(10) Pub. No.: US 2024/0294650 A1
(43) Pub. Date: Sep. 5, 2024

(54) ANTI-MERTK ANTIBODIES AND METHODS OF USE THEREOF

(71) Applicant: Alector LLC, South San Francisco, CA (US)

(72) Inventors: Michael KURNELLAS, San Mateo, CA (US); Santiago Viveros SALAZAR, San Carlos, CA (US); Marina ROELL, Concord, CA (US); Angie Grace YEE, San Francisco, CA (US); Seung-Joo LEE, South San Francisco, CA (US); Tina SCHWABE, San Francisco, CA (US); Maxime CHAPON, South San Francisco, CA (US); William Francis ESTACIO, Richmond, CA (US); Arnon ROSENTHAL, Woodside, CA (US)

(21) Appl. No.: 17/995,189

(22) PCT Filed: Mar. 30, 2021
(86) PCT No.: PCT/US2021/024970

§ 371 Date: Sep. 30, 2022

Related U.S. Application Data
(60) Provisional application No. 63/003,070, filed on Mar. 31, 2020.
(60) Provisional application No. 63/165,592, filed on Mar. 24, 2021.

(51) Int. Cl. C07K 16/28; G01N 33/573

(52) U.S. Cl. CPC C07K 16/2863; G01N 33/573; C07K 2317/33; C07K 2317/75; C07K 2317/76; C07K 2317/92; G01N 2333/9121

(57) ABSTRACT

The present disclosure is generally directed to antibodies, e.g., monoclonal, antibodies, antibody fragments, etc., that specifically bind a MerTK polypeptide, e.g. a mammalian MerTK or human MerTK, and use of such compositions in preventing, reducing risk, or treating a disease or disorder an individual in need thereof.

### CROSS-REFERENCE TO RELATED APPLICATIONS

This application claims the priority benefit of U.S. Provisional Application No. 63/003,070, filed Mar. 31, 2020, and U.S. Provisional Application No. 63/165,592, filed Mar. 24, 2021, each of which is herein incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 4503_011PC02_SL_ST25.TXT, date recorded: Mar. 30, 2021, size: 135,229 bytes).

### FIELD OF THE PRESENT DISCLOSURE

The present disclosure relates to anti-MerTK antibodies and uses (e.g., therapeutic uses) of such antibodies.

### BACKGROUND OF THE PRESENT DISCLOSURE

Mer Tyrosine Kinase (MerTK) belongs to the TAM (Tyro3, Axl, and MerTK) family of receptor tyrosine kinases. MerTK is a single-pass type 1 transmembrane protein with an extracellular domain having two immunoglobulin (Ig)-like and two fibronectin (FN) type III motifs (Graham et al, 2014, Nat Rev Cancer, 14:769-785; Rothlin et al, 2015, Annu Rev Immunol, 33:355-391).

Several ligands of MerTK have been identified, including Protein S (ProS or ProS1), Growth arrest specific gene 6 (Gas6), Tubby, Tubby-like protein 1 (TULP-1), and Galectin-3. MerTK transduces signals from the extracellular space via activation following ligand binding, leading to MerTK tyrosine auto-phosphorylation (Cummings et al, 2013, Clin Cancer Res, 19:5275-5280; Verma et al, 2011, Mol Cancer Ther, 10:1763-1773) and subsequent ERK and AKT-associated signal transduction.

MerTK has been identified as a multiple sclerosis (MS) susceptibility gene, and both rare and common variants lead to an increased risk of developing MS or altering disease course (Ma et al, 2011, PLoS ONE, 6:1-6; Binder et al, 2016, PLoS Genetics, pp. 1-25; Shen et al, 2021, Cell Reports, 34, 108835). MerTK regulates clearance of myelin debris by phagocytosis; efficient myelin debris clearance is a crucial step for tissue repair and remyelination. Inhibition or loss of MerTK reduced myelin phagocytosis (Healy et al, 2016, J Immunol, 196:3375-3384; Healy et al, 2017, Neurol Neuroimmunol Neuroinflamm, 4:e402; Tondo et al, 2019, Disease Markers, ID2387614:1-13; Weinger et al, 2009, Neurobiology, 175:283-293; Sharit-Zagardo et al, 2018, Pharmacol Ther, 188:97-117; Shen et al, 2021, Cell Reports, 34:108835). Additionally, mutations in MerTK reduced the ability of retinal pigment epithelial (RPE) cells to phagocytose photoreceptor outer segments, leading to the accumulation of debris separating photoreceptor cells from RPE cells, resulting in their degeneration and subsequent loss of vision (Lorach et al, 2018, Nature Scientific Reports, 8:11312).

There is a need for novel therapeutic anti-MerTK antibodies that are effective at treating or preventing autoimmune disorders (e.g., multiple sclerosis) and disorders associated with retinal ganglion degeneration. The present disclosure meets this need by providing anti-MerTK antibodies that agonize MerTK activity, including increasing phagocytosis.

All references cited herein, including patent applications and publications, are hereby incorporated by reference in their entirety.

### SUMMARY OF THE PRESENT DISCLOSURE

The present disclosure is generally directed to anti-Mer Tyrosine Kinase (MerTK) antibodies and methods of using such antibodies. The methods provided herein find use in preventing or treating an autoimmune disorder, such as for example, multiple sclerosis in an individual. In some aspects, the present disclosure provides a method for treating an autoimmune disorder (e.g., multiple sclerosis) in an individual, the method comprising administering to the individual in need thereof a therapeutically effective amount of an anti-MerTK antibody.

In one aspect, the present disclosure relates to an isolated anti-MerTK antibody that binds to a MerTK protein, wherein the antibody comprises a heavy chain variable region and a light chain variable region, wherein the heavy chain variable region comprises: an HVR-H1 comprising an amino acid sequence selected from the group consisting of SEQ ID NOs: 63, 64, 65, 66, 67, 68, 69, 70, 71, 72, 73, 74, 75, 76, 77, 78, 79, 80, 213, 214, and 224; an HVR-H2 comprising an amino acid sequence selected from the group consisting of SEQ ID NOs: 81, 82, 83, 84, 85, 86, 87, 88, 89, 90, 91, 92, 93, 94, 95, 96, 97, 98, 99, 100, 101, 102, 103, 104, 105, 106, 107, 108, 109, 215, 216, and 225; and an HVR-H3 comprising an amino acid sequence selected from the group consisting of SEQ ID NOs: 110, 111, 112, 113, 114, 115, 116, 117, 118, 119, 120, 121, 122, 123, 124, 125, 126, 127, 128, 129, 130, 131, 132, 133, 134, 135, 136, 217, 218, and 226; and the light chain variable region comprises: an HVR-L1 comprising an amino acid sequence selected from the group consisting of SEQ ID NOs: 137, 138, 139, 140, 141, 142, 143, 144, 145, 146, 147, 148, 149, 150, 151, 152, 153, 154, 155, 156, 157, 158, 159, 160, 161, 162, and 220; an HVR-L2 comprising an amino acid sequence selected from the group consisting of SEQ ID NOs: 163, 164, 165, 166, 167, 168, 169, 170, 171, 172, 173, 174, 175, 176, 177, 178, 179, 180, 181, 182, 183, 184, and 227; and an HVR-L3 comprising an amino acid sequence selected from the group consisting of SEQ ID NOs: 185, 186, 187, 188, 189, 190, 191, 192, 193, 194, 195, 196, 197, 198, 199, 200, 201, 202, 203, 204, 205, 206, 207, 208, 219, 221, and 228.

In one aspect, the present disclosure relates to an isolated anti-MerTK antibody that binds to a MerTK protein, wherein the antibody comprises an HVR-H1, HVR-H2, HVR-H3, HVR-L1, HVR-L2, and HVR-L3 comprising the amino acid sequences of (i) SEQ ID NOs: 63, 81, 110, 137, 163, and 185, respectively; (ii) SEQ ID NOs: 64, 82, 111, 138, 164, 186, respectively; (iii) SEQ ID NOs: 65, 83, 112, 139, 165, 187, respectively; (iv) SEQ ID NOs: 66, 84, 113, 138, 164, 188, respectively; (v) SEQ ID NOs: 224, 225, 226, 146, 227, and 228 respectively; (vi) SEQ ID NOs: 67, 85, 114, 140, 166, and 189, respectively; (vii) SEQ ID NOs: 68, 86, 115, 141, 167, and 190, respectively; (viii) SEQ ID NOs: 65, 87, 116, 142, 168, and 191, respectively; (ix) SEQ ID NOs: 69, 88, 117, 143, 169, and 192, respectively; (x) SEQ ID NOs: 70, 89, 118, 144, 163, and 193, respectively; (xi) SEQ ID NOs: 71, 90, 119, 145, 170, 194, respectively; (xii) SEQ ID NOs: 72, 91, 120, 146, 171, 195, respectively; (xiii) SEQ ID NOs: 73, 92, 121, 147, 172, 196, respectively; (xiv) SEQ ID NOs: 65, 93, 122, 148, 173, 197, respectively; (xv) SEQ ID NOs: 66, 94, 123, 149, 174, 198, respectively; (xvi) SEQ ID NOs: 66, 95, 124, 150, 164, 188, respectively; (xvii) SEQ ID NOs: 73, 96, 125, 151, 175, and 199, respectively; (xviii) SEQ ID NOs: 74, 97, 126, 152, 176, and 200, respectively; (xix) SEQ ID NOs: 71, 98, 127, 153, 177, and 201, respectively; (xx) SEQ ID NOs: 66, 99, 128, 138, 164, and 188, respectively; (xxi) SEQ ID NOs: 75, 100, 129, 154, 178, and 202, respectively; (xxii) SEQ ID NOs: 71, 101, 130, 155, 179, and 201, respectively; (xxiii) SEQ ID NOs: 76, 102, 131,155, 179, 201, respectively; (xxiv) SEQ ID NOs: 77, 103, 132, 157, 181, and 204, respectively; (xxv) SEQ ID NOs: 78, 104, 133, 158, 182, and 205, respectively; (xxvi) SEQ ID NOs: 74, 105, 126, 159, 176, and 200, respectively; (xxvii) SEQ ID NOs: 79, 106, 134, 160, 183, and 206, respectively; (xxviii) SEQ ID NOs: 74, 107, 126, 161, 176, and 200, respectively; (xxix) SEQ ID NOs: 70, 108, 135, 144, 170, and 207, respectively; (xxx) SEQ ID NOs: 80, 109, 136, 162, 184, and 208, respectively; (xxxi) SEQ ID NOs: 213, 215, 217, 156, 180, and 219, respectively; or (xxxii) SEQ ID NOs: 214, 216, 218, 220, 172, and 221, respectively.

In some aspects that may be combined with any of the aspects provided herein, an anti-MerTK antibody of the present disclosure is an isolated antibody that binds to a MerTK protein, wherein the antibody comprises the HVR-H1, HVR-H2, HVR-H3, HVR-L1, HVR-L2 and HVR-L3 sequences of MTK-201, MTK-202, MTK-203, MTK-204, MTK-205, MTK-206, MTK-207, MTK-208, MTK-209, MTK-210, MTK-211, MTK-212, MTK-213, MTK-214, MTK-215, MTK-216, MTK-217, MTK-218, MTK-219, MTK-220, MTK-221, MTK-222, MTK-223, MTK-224, MTK-225, MTK-226, MTK-227, MTK-228, MTK-229, MTK-230, MTK-231, or MTK-232 antibody. In some aspects, the HVRs are the Kabat-defined HVRs, the Chothia-defined HVRs, or the AbM-defined HVRs.

In some aspects that may be combined with any of the aspects provided herein, an anti-MerTK antibody of the present disclosure is an isolated antibody that binds to a MerTK protein, wherein the antibody comprises a heavy chain variable region, wherein the heavy chain variable region comprises an amino acid sequence selected from the group consisting of SEQ ID NOs:5, 6, 7, 8, 9, 10, 11, 12, 13, 14, 15, 16, 17, 18, 19, 20, 21, 22, 23, 24, 25, 26, 27, 28, 29, 30, 31, 32, 33, 209, 210, and 222.

In some aspects that may be combined with any of the aspects provided herein, an anti-MerTK antibody of the present disclosure is an isolated antibody that binds to a MerTK protein, wherein the antibody comprises a light chain variable region, wherein the light chain variable region comprises an amino acid sequence selected from the group consisting of SEQ ID NOs: 34, 35, 36, 37, 38, 39, 40, 41, 42, 43, 44, 45, 46, 47, 48, 49, 50, 51, 52, 53, 54, 55, 56, 57, 58, 59, 60, 61, 62, 211, 212, and 223.

In some aspects that may be combined with any of the aspects provided herein, an anti-MerTK antibody of the present disclosure is an isolated antibody that binds to a MerTK protein, wherein the antibody comprises a heavy chain variable region and a light chain variable region, wherein the heavy chain variable region comprises an amino acid sequence selected from SEQ ID NOs: 5, 6, 7, 8, 9, 10, 11, 12, 13, 14, 15, 16, 17, 18, 19, 20, 21, 22, 23, 24, 25, 26, 27, 28, 29, 30, 31, 32, 33, 209, 210, and 222 and the light chain variable region comprises an amino acid sequence selected from SEQ ID NOs: 34, 35, 36, 37, 38, 39, 40, 41, 42, 43, 44, 45, 46, 47, 48, 49, 50, 51, 52, 53, 54, 55, 56, 57, 58, 59, 60, 61, 62, 211, 212, 223.

In some aspects that may be combined with any of the aspects provided herein, an anti-MerTK antibody of the present disclosure is an isolated antibody that binds to a MerTK protein, wherein the antibody a heavy chain variable region and a light chain variable region comprising the amino acid sequences of SEQ ID NOs:5 and 34, respectively; SEQ ID NOs:6 and 35, respectively; SEQ ID NOs:7 and 36, respectively; respectively; SEQ ID NOs:8 and 37, respectively; SEQ ID NOs:222 and 223, respectively; SEQ ID NOs:9 and 38, respectively; SEQ ID NOs:10 and 39, respectively; SEQ ID NOs:11 and 40, respectively; SEQ ID NOs:12 and 41, respectively; SEQ ID NOs:13 and 42, respectively; SEQ ID NOs:14 and 43, respectively; SEQ ID NOs:15 and 44, respectively; SEQ ID NOs:16 and 45, respectively; SEQ ID NOs:17 and 46, respectively; SEQ ID NOs:18 and 47, respectively; SEQ ID NOs:19 and 48, respectively; SEQ ID NOs:20 and 49, respectively; SEQ ID NOs:21 and 50, respectively; SEQ ID NOs:22 and 51, respectively; SEQ ID NOs:23 and 52, respectively; SEQ ID NOs:24 and 53, respectively; SEQ ID NOs:25 and 54, respectively; SEQ ID NOs:26 and 55, respectively; SEQ ID NOs:27 and 56, respectively; SEQ ID NOs:28 and 57, respectively; SEQ ID NOs:29 and 58, respectively; SEQ ID NOs:30 and 59, respectively; SEQ ID NOs:31 and 60, respectively; SEQ ID NOs:32 and 61, respectively; SEQ ID NOs:33 and 62, respectively; SEQ ID NOs:209 and 211, respectively; and SEQ ID NOs:210 and 212, respectively.

In one aspect, the present disclosure relates to an isolated antibody that binds to a MerTK protein, wherein the antibody competitively inhibits binding of one or more of the antibodies of any of the aspects herein for binding to MerTK.

In another aspect, the present disclosure relates to an isolated antibody that binds to a MerTK protein, wherein the antibody binds essentially the same or an overlapping epitope on MerTK as an antibody of any of the aspects herein. In another aspect, the present disclosure relates to an isolated antibody that binds to a MerTK protein, wherein the antibody binds the same epitope on MerTK as an antibody of any of the aspects herein.

In certain aspects that may be combined with any of the aspects herein, the MerTK protein is a mammalian protein or a human protein. In certain aspects that may be combined with any of the aspects herein, the MerTK protein is a wild-type protein. In certain aspects that may be combined with any of the aspects herein, the MerTK protein is a naturally occurring variant. In certain aspects that may be combined with any of the aspects herein, an anti-MerTK antibody binds to human MerTK and to cynomolgus monkey MerTK and/or murine MerTK.

In some aspects that may be combined with any of the aspects herein, an anti-MerTK antibody of the present disclosure does not inhibit or reduce binding of one or more ligands to MerTK. In some aspects that may be combined with any of the aspects herein, an anti-MerTK antibody of the present disclosure does not inhibit or reduce binding of ProS to MerTK. In some aspects that may be combined with any of the aspects herein, an anti-MerTK antibody of the present disclosure does not inhibit or reduce binding of Gas6 to MerTK. In some aspects that may be combined with any of the aspects herein, an anti-MerTK antibody of the present disclosure does not inhibit or reduce binding of Gas6 to MerTK and does not inhibit or reduce binding of ProS to MerTK.

In some aspects that may be combined with any of the aspects provided herein, an anti-MerTK antibody of the present disclosure increases phagocytosis. In some aspects that may be combined with any of the aspects provided herein, an anti-MerTK antibody of the present disclosure increases clearance of myelin debris by phagocytosis. In some aspects that may be combined with any of the aspects provided herein, an anti-MerTk antibody of the present disclosure increases photoreceptor outer segment phagocytosis by retinal pigment epithelial cells.

In some aspects that may be combined with any of the aspects provided herein, an anti-MerTK antibody of the present disclosure does not reduce efferocytosis by more than 40%. In some aspects that may be combined with any of the aspects provided herein, an anti-MerTK antibody of the present disclosure does not reduce efferocytosis by more than 30%. In some aspects that may be combined with any of the aspects provided herein, an anti-MerTK antibody of the present disclosure does not reduce efferocytosis by more than 20%. In some aspects that may be combined with any of the aspects provided herein, an anti-MerTK antibody of the present disclosure does not reduce efferocytosis by more than 10%.

In some aspects that may be combined with any of the aspects provided herein, an anti-MerTK antibody of the present disclosure increases phosphorylation of MerTK in the absence of Gas6. In some aspects that may be combined with any of the aspects provided herein, an anti-MerTK antibody of the present disclosure increases phosphorylation of MerTK in the presence of Gas6.

In some aspects that may be combined with any of the aspects provided herein, an anti-MerTK antibody of the present disclosure increases phosphorylation of protein kinase B (AKT).

In some aspects that may be combined with any of the aspects provided herein, an anti-MerTK antibody of the present disclosure increases monocyte chemoattractant protein-1 (MCP-1) expression in macrophages. In some aspects that may be combined with any of the aspects provided herein, an anti-MerTK antibody of the present disclosure increases MCP-1 expression in macrophages in the presence of ProS. In some aspects that may be combined with any of the aspects provided herein, an anti-MerTK antibody of the present disclosure increases MCP-1 expression in macrophages in the absence of ProS.

In some aspects that may be combined with any of the aspects provided herein, an anti-MerTK antibody of the present disclosure binds to the N-terminal domain of MerTK, Ig-like domain 1, Ig-like domain 2, fibronectin type III domain 1, fibronectin type III domain 2, and/or juxtamembrane domain of MerTK.

In some aspects that may be combined with any of the aspects provided herein, an anti-MerTK antibody of the present disclosure binds to cynomolgus MerTK, but not murine MerTK, binds to murine MerTK, but not cynomolgus MerTK, or binds to cynomolgus and murine MerTK. In some aspects that may be combined with any of the aspects provided herein, an anti-MerTK antibody of the present disclosure binds human MerTK with an affinity of less than 440 nM, less than 400 nM, less than 350 nM, less than 300 nM, less than 250 nM, less than 200 nM, less than 150 nM, less than 100 nM, or less than 50 nM.

In some aspects that may be combined with any of the aspects provided herein, an anti-MerTK antibody of the present disclosure binds Gas6 in the absence of MerTK. In some aspects that may be combined with any of the aspects provided herein, an anti-MerTK antibody of the present disclosure binds ProS in the absence of MerTK.

In some aspects that may be combined with any of the aspects herein, the anti-MerTK antibody of the present disclosure is a monoclonal antibody. In some aspects that may be combined with any of the aspects herein, the antibody is a human antibody. In some aspects that may be combined with any of the aspects herein, the antibody is a humanized antibody. In some aspects that may be combined with any of the aspects herein, the antibody is a bispecific antibody. In some aspects that may be combined with any of the aspects herein, the antibody is a multivalent antibody. In some aspects that may be combined with any of the aspects herein, the antibody is a chimeric antibody.

In some aspects that may be combined with any of the aspects herein, the anti-MerTK antibody of the present disclosure is of the IgG class, the IgM class, or the IgA class. In some aspects, the antibody is of the IgG class and has an IgG1, IgG2, or IgG4 isotype. In certain aspects that may be combined with any of the aspects herein, the antibody is a full-length antibody. In certain aspects that may be combined with any of the aspects herein, the antibody is an antibody fragment. In certain aspects that may be combined with any of the aspects herein, the antibody is an antibody fragment that binds to an epitope on human MerTK or a mammalian MerTK protein. In certain aspects that may be combined with any of the aspects herein, the antibody fragment is a Fab, Fab′, Fab′-SH, F(ab′)2, Fv, or scFv fragment.

In another aspect, the present disclosure relates to an isolated nucleic acid comprising a nucleic acid sequence encoding an anti-MerTK antibody of any of the preceding aspects. In some aspects, the present disclosure relates to a vector comprising the nucleic acid of any of the preceding aspects. In some aspects, the present disclosure relates to an isolated host cell comprising the nucleic acid of any of the preceding aspects or the vector of any of the preceding aspects. In some aspects, the present disclosure relates to an isolated host cell comprising (i) a nucleic acid comprising a nucleic acid sequence encoding the VH of an anti-MerTK antibody of any of the preceding aspects and (ii) a nucleic acid comprising a nucleic acid sequence encoding the VL of the anti-MerTK antibody.

In another aspect, the present disclosure relates to a method of producing an antibody that binds to human MerTK antibody, comprises culturing the host cell of any of the preceding aspects so that the anti-MerTK antibody is produced. In certain aspects, the method further comprises recovering the anti-MerTK antibody produced by the cell.

In another aspect, the present disclosure relates to a pharmaceutical composition comprises an anti-MerTK antibody of any one of the preceding aspects and a pharmaceutically acceptable carrier.

In one aspect, the present disclosure relates to a method of detecting MerTk in a sample comprising contacting said sample with an anti-MerTK antibody of any of the preceding aspects, optionally wherein the method further comprises detecting the binding of the antibody to MerTK in the sample.

It is to be understood that one, some, or all of the properties of the various aspects described herein may be combined to form other aspects of the present disclosure. These and other aspects of the disclosure will become apparent to one of skill in the art. These and other aspects of the disclosure are further described by the detailed description that follows.

### BRIEF DESCRIPTION OF THE DRAWINGS

FIG. 1 sets forth data showing phosphorylation of MerTK (pMerTK) in human macrophages following addition of anti-MerTK antibodies of the present disclosure.

FIG. 2 sets forth data showing equilibrium dissociation constants (KD) of anti-MerTK antibodies of the present disclosure.

FIGS. 3A, 3B, and 3C set forth data showing the effect of anti-MerTK antibodies of the present disclosure on MCP-1 expression in macrophages.

FIG. 4 sets forth data showing the effect of anti-MerTK antibodies of the present disclosure on tyrosine phosphorylation of MerTK (pMerTK) in the presence or absence of Gas6 protein.

### DETAILED DESCRIPTION OF THE PRESENT DISCLOSURE

The present disclosure relates to anti-MerTK antibodies (e.g., monoclonal antibodies); methods of making and using such antibodies; pharmaceutical compositions comprising such antibodies; nucleic acids encoding such antibodies; and host cells comprising nucleic acids encoding such antibodies.

The techniques and procedures described or referenced herein are generally well understood and commonly employed using conventional methodology by those skilled in the art, such as, for example, the widely utilized methodologies such as those described in Sambrook et al. Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003); Monoclonal Antibodies: A Practical Approach (P. Shepherd and C. Dean, eds., Oxford University Press, 2000).

### I. Definitions

The terms “MerTK” or “MerTK polypeptide” or “MerTK protein” are used interchangeably herein refer herein to any native MerTK from any vertebrate source, including mammals such as primates (e.g., humans and cynomolgus monkeys (cynos)) and rodents (e.g., mice and rats), unless otherwise indicated. MerTK is also referred to as c-mer, MER, Proto-oncogene c-Mer, Receptor Tyrosine Kinase MerTK, Tyrosine-protein Kinase Mer, STK Kinase, RP38, and MGC133349. In some aspects, the term encompasses both wild-type sequences and naturally occurring variant sequences, e.g., splice variants or allelic variants. In some aspects, the term encompasses “full-length,” unprocessed MerTK as well as any form of MerTK that results from processing in the cell. In some aspects, the MerTK is human MerTK. As used herein the term “human MerTK” refers to a polypeptide with the amino acid sequence of SEQ ID NO:1.

The terms “anti-MerTK antibody,” “MerTK antibody,” an “antibody that binds to MerTK,” and “antibody that specifically binds MerTK” refer to an antibody that is capable of binding MerTK with sufficient affinity such that the antibody is useful as a diagnostic and/or therapeutic agent in targeting MerTK. In one aspect, the extent of binding of an anti-MerTK antibody to an unrelated, non-MerTK polypeptide is less than about 10% of the binding of the antibody to MerTK as measured, e.g., by a radioimmunoassay (RIA). In certain aspects, an antibody that binds to MerTK has a dissociation constant (KD) of <1 μM, <100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹ M to 10⁻¹³ M). In certain aspects, an anti-MerTK antibody binds to an epitope of MerTK that is conserved among MerTK from different species.

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of about any of 10⁻⁴ M or lower, 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value.

The term “immunoglobulin” (Ig) is used interchangeably with “antibody” herein. The term “antibody” herein is used in the broadest sense and specially covers monoclonal antibodies, polyclonal antibodies, multispecific antibodies (e.g., bispecific antibodies) including those formed from at least two intact antibodies, and antigen-binding antibody fragments so long as they exhibit the desired biological activity.

“Native antibodies” are usually heterotetrameric glycoproteins of about 150,000 Daltons, composed of two identical light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, C T, 1994, page 71 and Chapter 6.

The light chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”), and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example, Abbas et al., Cellular and Molecular Immunology, 4^{th} ed. (W.B. Saunders Co., 2000).

The “variable region” or “variable domain” of an antibody, such as an anti-MerTK antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-MerTK antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as a monoclonal anti-MerTK antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure can be made by a variety of techniques, including, but not limited to one or more of the following methods, immunization methods of animals including, but not limited to rats, mice, rabbits, guinea pigs, hamsters and/or chickens with one or more of DNA(s), virus-like particles, polypeptide(s), and/or cell(s), the hybridoma methods, B-cell cloning methods, recombinant DNA methods, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody, such as an anti-MerTK antibody of the present disclosure, in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” refers to a molecule other than an intact antibody that comprises a portion of an intact antibody that binds the antigen to which the intact antibody binds. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-MerTK antibodies of the present disclosure, produces two identical antigen-binding fragments, called “Fab” fragments, and a residual “Fc” fragment, a designation reflecting the ability to crystallize readily. The Fab fragment consists of an entire light chain along with the variable region domain of the heavy chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both heavy chains held together by disulfides. The effector functions of antibodies are determined by sequences in the Fc region, the region which is also recognized by Fc receptors (FcR) found on certain types of cells.

“Functional fragments” of antibodies, such as anti-MerTK antibodies of the present disclosure, comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

As used herein, a “chimeric antibody” refers to an antibody (immunoglobulin), such as a chimeric anti-MerTK antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include PRIMATIZED© antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as humanized forms of anti-MerTK antibodies of the present disclosure, are chimeric antibodies comprising amino acid residues from non-human HVRs and amino acid residues from human FRs. In certain aspects, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one that possesses an amino-acid sequence corresponding to that of an antibody, such as an anti-MerTK antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries and yeast-display libraries. Human antibodies can be prepared by administering the antigen to a transgenic animal that has been modified to produce such antibodies in response to antigenic challenge, but whose endogenous loci have been disabled, e.g., immunized xenomice as well as generated via a human B-cell hybridoma technology.

The term “hypervariable region,” “HVR,” or “HV,” when used herein refers to the regions of an antibody-variable domain, such as that of an anti-MerTK antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. Naturally occurring camelid antibodies consisting of a heavy chain only are functional and stable in the absence of light chain.

A number of HVR delineations are in use and are encompassed herein. In some aspects, the HVRs may be Kabat complementarity-determining regions (CDRs) based on sequence variability and are the most commonly used (Kabat et al., supra). In some aspects, the HVRs may be Chothia CDRs. Chothia refers instead to the location of the structural loops (Chothia and Lesk J. Mol. Biol. 196:901-917 (1987)). In some aspects, the HVRs may be AbM HVRs. The AbM HVRs represent a compromise between the Kabat CDRs and Chothia structural loops, and are used by Oxford Molecular's AbM antibody-modeling software. In some aspects, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred aspect) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may comprise pre-existing amino acid sequence changes. In some aspects, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one aspect, the VL acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the V_{L}, the subgroup may be subgroup kappa I, kappa II, kappa III or kappa IV as in Kabat et al., supra. Additionally, for the V_{H}, the subgroup may be subgroup I, subgroup II, or subgroup III as in Kabat et al., supra.

An “amino-acid modification” at a specified position, e.g., of an anti-MerTK antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

“Fv” is the minimum antibody fragment which comprises a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains, which enables the sFv to form the desired structure for antigen binding.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least 80% homology with a native sequence Fc region and/or with an Fc region of a parent polypeptide, and most preferably at least 90% homology therewith, more preferably at least 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

The term “compete” when used in the context of antibodies that compete for the same epitope or overlapping epitopes means competition between antibody as determined by an assay in which the antibody being tested prevents or inhibits (e.g., reduces) specific binding of a reference molecule (e.g., a ligand, or a reference antibody) to a common antigen (e.g., MerTK or a fragment thereof). Numerous types of competitive binding assays can be used to determine if antibody competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antibody and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit (e.g., reduce) specific binding of a reference antibody to a common antigen by at least 20%, 30%, 40%, 50%, 60%, 70%, 80%, 85%, 90%, 95%, 97.5%, and/or near 100%.

As used herein, an “interaction” between a MerTK polypeptide and a second polypeptide encompasses, without limitation, protein-protein interaction, a physical interaction, a chemical interaction, binding, covalent binding, and ionic binding. As used herein, an antibody “inhibits interaction” between two polypeptides when the antibody disrupts, reduces, or completely eliminates an interaction between the two polypeptides. An antibody of the present disclosure, thereof, “inhibits interaction” between two polypeptides when the antibody thereof binds to one of the two polypeptides. In some aspects, the interaction can be inhibited by at least any of 20%, 30%, 40%, 50%, 60%, 70%, 80%, 85%, 90%, 95%, 97.5%, and/or near 100%.

The term “epitope” includes any determinant capable of being bound by an antibody. An epitope is a region of an antigen that is bound by an antibody that targets that antigen, and when the antigen is a polypeptide, includes specific amino acids that directly contact the antibody. Most often, epitopes reside on polypeptides, but in some instances, can reside on other kinds of molecules, such as nucleic acids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of polypeptides and/or macromolecules.

An “isolated” antibody, such as an isolated anti-MerTK antibody of the present disclosure, is one that has been identified, separated and/or recovered from a component of its production environment (e.g., naturally or recombinantly). Preferably, the isolated antibody is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred aspects, the antibody will be purified: (1) to greater than 95% by weight of antibody as determined by, for example, the Lowry method, and in some aspects, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T-cells since at least one component of the antibody's natural environment will not be present. Ordinarily, however, an isolated polypeptide or antibody will be prepared by at least one purification step.

An “isolated” nucleic acid molecule encoding an antibody, such as an anti-MerTK antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure.

“Carriers” as used herein include pharmaceutically acceptable carriers, excipients, or stabilizers that are nontoxic to the cell or mammal being exposed thereto at the dosages and concentrations employed.

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some aspects, the individual is human.

The term “about” as used herein refers to the usual error range for the respective value readily known to the skilled person in this technical field. In some aspects when “about” is used to modify a numberic value or numeric range, the term indicates that deviations of up t 10% above and down to 10% below the value or range remain within the intended meaning of the recited value or range. Reference to “about” a value or parameter herein includes (and describes) aspects that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspects of the present disclosure described herein include “comprising,” “consisting,” and “consisting essentially of” aspects.

### II. Anti-MerTK Antibodies

Provided herein are anti-MerTK antibodies. Antibodies provided herein are useful, e.g., for the diagnosis or treatment of the MerTK associated disorders. Provided herein are anti-MerTK antibodies.

In one aspect, the present disclosure provides isolated (e.g., monoclonal) antibodies that bind to an epitope within a MerTK protein or polypeptide of the present disclosure. MerTK proteins or polypeptides of the present disclosure include, without limitation, a mammalian MerTK protein or polypeptide, human MerTK protein or polypeptide, mouse (murine) MerTK protein or polypeptide, and cynomolgus (cyno) MerTK protein or polypeptide. MerTK proteins and polypeptides of the present disclosure include naturally occurring variants of MerTK. In some aspects, MerTK proteins and polypeptides of the present disclosure are membrane bound. In some aspects, MerTK proteins and polypeptides of the present disclosure are a soluble extracellular domain of MerTK.

In some aspects, MerTK is expressed in a cell. In some aspects, MerTK is expressed in phagocytic cells, including without limitation, macrophages, dendritic cells, or microglia. In some aspects, MerTK is expressed in microglia. In some aspects, MerTK is expressed in astrocytes, monocytes, natural killer cells, natural killer T cells, endothelial cells, megakaryocytes, and platelets. In some aspects, high levels of MerTK expression are also found in ovary, prostate, testis, lung, retina, and kidney.

### Antibody Activities

In some aspects, an anti-MerTK antibody is provided that binds to human MerTK but does not bind to cyno MerTK. In some aspects, an anti-MerTK antibody is provided that binds to human MerTK but does not bind to murine MerTK. In some aspects, an anti-MerTK antibody is provided that binds to human MerTK but does not bind to cyno MerTK and does not bind to murine MerTK. In some aspects, an anti-MerTK antibody is provided that binds to human MerTK and binds to cyno MerTK. In some aspects, an anti-MerTK antibody is provided that binds to human MerTK and binds to cyno MerTK but does not bind to murine MerTK. In some aspects, an anti-MerTK antibody is provide that binds to human MerTK and binds to murine MerTK. In some aspects, an anti-MerTK antibody is provide that binds to human MerTK and binds to murine MerTK but does not bind cyno MerTK. In some aspects, an anti-MerTK antibody is provide that binds to human MerTK, binds to murine MerTK, and binds cyno MerTK.

### MerTK Binding Partners

MerTK proteins of the present disclosure interact with (e.g., bind) one or more ligands or binding partners, including, without limitation, Protein S (ProS or ProS1), Growth arrest specific gene 6 (Gas6), Tubby, Tubby-like protein 1 (TULP-1), and Galectin-3. Anti-MerTK antibodies of the present disclosure can affect the interaction of MerTK with one or more of its various ligands and binding partners.

Anti-MerTK antibodies of the present disclosure do not block or inhibit binding of Gas6 ligand and/or of ProS ligand binding to MerTK. Accordingly, in some aspects, an anti-MerTK antibody of the present disclosure does not inhibit or reduce binding between MerTK and one or more MerTK ligands. In some aspects, an anti-MerTK antibody of the present disclosure does not inhibit or reduce binding of ProS to MerTK. In some aspects, an anti-MerTK antibody of the present disclosure does not reduce binding of ProS to MerTK by more than 30%. In some aspects, an anti-MerTK antibody of the present disclosure does not reduce binding of ProS to MerTK by more than 20%. In some aspects, an anti-MerTK antibody of the present disclosure does not reduce binding of ProS to MerTK by more than 10%. In some aspects, an anti-MerTK antibody of the present disclosure does not reduce binding of ProS to MerTK by more than 5%. In some aspects, an anti-MerTK antibody of the present disclosure does not inhibit or reduce binding of Gas6 to MerTK. In some aspects, an anti-MerTK antibody of the present disclosure does not reduce binding of Gas6 to MerTK by more than 30%. In some aspects, an anti-MerTK antibody of the present disclosure does not reduce binding of Gas6 to MerTK by more than 20%. In some aspects, an anti-MerTK antibody of the present disclosure does not reduce binding of Gas6 to MerTK by more than 10%. In some aspects, an anti-MerTK antibody of the present disclosure does not reduce binding of Gas6 to MerTK by more than 5%. In some aspects, an anti-MerTK antibody of the present disclosure does not inhibit or reduce binding of Gas6 to MerTK and does not inhibit or reduce binding of ProS to MerTK. In some aspects, an anti-MerTK antibody of the present disclosure does not reduce binding of ProS to MerTK by more than 30% and does not reduce binding of Gas6 to MerTK by more than 30%. In some aspects, an anti-MerTK antibody of the present disclosure does not reduce binding of ProS to MerTK by more than 20% and does not reduce binding of Gas6 to MerTK by more than 20%. In some aspects, an anti-MerTK antibody of the present disclosure does not reduce binding of ProS to MerTK by more than 10% and does not reduce binding of Gas6 to MerTK by more than 10%. In some aspects, an anti-MerTK antibody of the present disclosure does not reduce binding of ProS to MerTK by more than 5% and does not reduce binding of Gas6 to MerTK by more than 5%. In some aspects, an anti-MerTK antibody of the present disclosure does not inhibit or reduce Gas6 ligand binding and/or does not inhibit or reduce ProS ligand binding to MerTK in vitro.

Further provided herein are methods of screening for anti-MerTK antibodies that bind MerTK and that do not block or reduce the interactions between MerTK and one or more MerTK ligands or binding partners.

### Efferocytosis

Efferocytosis refers to phagocytic clearance of dying or apoptotic cells. Efferocytosis can be accomplished by professional phagocytes (e.g., macrophages, dendritic cells, microglia), non-professional phagocytes (e.g., epithelial cells, fibroblasts, retinal pigment epithelial cells), or specialized phagocytes. (Elliott et al, 2017, J Immunol, 198:1387-1394.) Efferocytosis leads to the removal of dead or dying cells before their membrane integrity is breached and their cellular contents leak into the surrounding tissue, thus preventing exposure of tissue to toxic enzymes, oxidants, and other intracellular components.

Apoptotic cells expose a variety of molecules on their cell surface (“eat-me” signals) that are recognized by receptors on phagocytic cells. One such “eat me” signaling molecules is phosphatidylserine (PtdSer), which is normally confined to the inner leaflet of the cell membrane. During apoptosis, PtdSer is exposed to the outer leaflet of the cell membrane. MerTK ligands ProS and Gas6 contain gamma-carboxylated glutamic acid residues near their N-terminal domains; gamma-carboxylation of the glutamic acid domain enables binding to phosphatidylserine. Gas6 or ProS bind to PtdSer on apoptotic cells and simultaneously bind MerTK on phagocytes. Such ligand engagement with MerTK activates efferocytosis.

The ability of an antibody to block (or not block) efferocytosis can be determined, e.g., using the methods in Example 7 herein. For instance, an efferocytosis assay can comprise (i) adding apoptotic cells to phagocytic cells that have been exposed or not exposed to an antibody or exposed to a test antibody and a negative control antibody and (ii) determining the uptake of the apoptotic cells by the phagocytic cells. The phagocytic cells can be professional phagocytes or non-professional phagocytes as discussed above. In some aspects, the phagocytic cells are macrophages. In some aspects, the phagocytic cells (e.g., macrophages) are starved (e.g., for about an hour) prior to the exposure to the antibody and/or the apoptotic cells. In some aspects, the phagocytic cells (e.g., macrophages) are incubated with the antibody for about 5 minutes to about an hour (e.g., for about 30 minutes) prior to the exposure to the apoptotic cells, e.g., at about 37° C. The apoptotic cells can be, e.g., Jurkat cells that were treated with an apoptosis-inducing agent such as 1 μM staurosporin (SigmaAldrich). The apoptotic cells can be labeled cells (e.g., dyed cells). In some aspects, the apoptotic cells are exposed to the phagocytic cells (e.g., macrophages) for about an hour. An antibody that does not block efferocytosis does not significantly increase the uptake of apoptotic cells in such an assay as compared to the uptake in the absence of the antibody or in the presence of a negative control antibody. In some aspects, an anti-MerTK antibody provided herein antibody does not diminish efferocytosis by more than 50%. In some aspects, an anti-MerTK antibody provided herein antibody does not diminish efferocytosis by more than 45%. In some aspects, an anti-MerTK antibody provided herein antibody does not diminish efferocytosis by more than 40%. In some aspects, an anti-MerTK antibody provided herein antibody does not diminish efferocytosis by more than 35%. In some aspects, an anti-MerTK antibody provided herein antibody does not diminish efferocytosis by more than 30%. In some aspects, an anti-MerTK antibody provided herein antibody does not diminish efferocytosis by more than 25%. In some aspects, an anti-MerTK antibody provided herein antibody does not diminish efferocytosis by more than 20%. In some aspects, an anti-MerTK antibody provided herein antibody does not diminish efferocytosis by more than 15%. In some aspects, an anti-MerTK antibody provided herein antibody does not diminish efferocytosis by more than 10%. In some aspects, an anti-MerTK antibody provided herein antibody does not diminish efferocytosis by more than 5%.

### Phagocytosis

Phagocytosis refers to the process by which phagocytes ingest or engulf apoptotic cells, particles, or cell debris. Within the central nervous system, phagocytosis is a critical process required for proper neural circuit development and maintaining homeostasis. Destruction of myelin sheathes within the CNS, as occurs in multiple sclerosis, produces degenerating myelin at site of injury and inflammation. The resulting myelin debris must be cleared through phagocytosis from sites of injury to promote repair. Studies utilizing human macrophages and microglia have demonstrated that MerTK is an essential phagocytic receptor for myelin, expression of MerTK correlates with myelin phagocytosis in vitro, and MerTK levels are reduced in MS patient macrophages (Healy et al, 2016, J Immunol, 196:3375-3384; Healy et al, 2017, Neurol Neuroimmunol Neuroinflamm, 4:e402; Galloway et al, 2019, Front Immunol, 10:article 790).

The ability of an antibody to increase phagocytosis can be determined using assays known in the art (see e.g., Healy et al, 2016, J Immunol, 196:3375-3384). For example, a phagocytosis assay can comprise (i) adding myelin to cells (e.g. myeloid cells) in the presence and the absence of an antibody or in the presence of the test antibody and a negative control antibody, and (ii) determining the uptake of the myelin by the cells. The cells can be plated cells (e.g., plated myeloid cells). The cells (e.g. myeloid cells) can be polarized. (See e.g., Durafourt et al, 2012, Glia 60:717-727.) The myelin can be labeled. For example, the myelin can be dyed using, e.g., a pH-sensitive dye such as ph-Rhodamine (Invitrogen). In order to obtain dyed myelin, myelin and a dye (e.g., a pH-sensitive dye) can be incubated, e.g., for about 1 hour, optionally in PBS. The myelin (e.g. dyed myelin) can be added to the cells (e.g., myeloid cells) to a final concentration of, e.g., about 5 μg/ml to about 20 μg/ml. Thus, in some aspects, myelin (e.g. dyed myelin) is added to the cells (e.g., myeloid cells) to a final concentration of about 5 μg/ml or about 20 μg/ml. An antibody that increases phagocytosis of myelin (i.e., increases clearance of myelin by phagocytosis) increases the uptake of myelin by cells in such an assay as compared to the uptake in the absence of the antibody or in the presence of a negative control antibody.

### Macrophage Chemoattractant Protein-1

Monocyte chemoattractant protein-1 (MCP-1) recruits immune cells to sites of injury. Anti-MerTK antibodies of the present disclosure increased MCP-1 levels in M2c-polarized macrophages, indicating that the anti-MerTk antibodies are effective at activating or increasing the activity of MerTK and thus effective at enhancing phagocytosis and efferocytosis.

pAKT

The protein kinase B (AKT) signaling pathway is a signal transduction pathway that promotes cell survival and growth, a process initiated by phosphorylation of AKT (pAKT). MerTK ligand Gas6 increases pAKT levels in cells. Anti-MerTK antibodies of the present disclosure increased pAKT levels in cells in the absence of Gas6. In some aspects, an anti-MerTK antibody of the present disclosure increases pAKT levels by at least 1-fold, by at least 2-fold, by at least 3-fold, or by at least 4-fold.

pMerTK

MerTK ligand Gas6 increases phosphorylation of MerTK (pMerTK). Anti-MerTK antibodies of the present disclosure increased phosphorylation of MerTK (pMerTK) in the absence of Gas6. Additionally, anti-MerTk antibodies of the present disclosure were effective at increasing pMerTK to levels higher than observed with Gas6 alone. In some aspects, an anti-MerTK antibody of the present disclosure increases pMerTK levels by at least 1-fold, by at least 2-fold, by at least 3-fold, or by at least 4-fold.

Accordingly, in some aspects, an anti-MerTK antibody of the present disclosure increases MerTK activity, including by not limited to increasing phagocytosis by a phagocytic cell, does not reduce efferocytosis by more than 40%, increases pMerTK levels, increases pAKT levels, and increases MCP-1 expression, or any combination thereof. Not wishing to be bound by theory, increased activity of MerTK by anti-MetTK antibodies of the present disclosure allows for increased ability of MerTK to interact with its binding proteins (e.g., Gas6, ProS) to signal microglia and astrocytes to enhance phagocytosis of degraded myelin, enhance their migration to sites where myelin regeneration is needed, and enhance survival and/or proliferation of microglia.

Membrane-bound MerTK has been shown to be proteolytically cleaved, leading to formation of soluble MerTK (sMerTK), which has been shown to inhibit thrombocytosis in mice and inhibit efferocytosis in vitro. MerTK is cleaved by metalloproteinases (e.g., ADAM17, ADAM10) at proline 485 in mice macrophages. (Thorp et al, 2011, J. Biol. Chem., 38:33335-33344). Not wishing to be bound by theory, reduction of MerTK cleavage by an anti-MerTK antibody of the present disclosure is an effective means to reduce the formation of sMerTK and thus maintain or increase MerTK activity and signaling, resulting in increased phagocytosis and efferocytosis activity.

### A. Exemplary Antibodies and Certain Other Antibody Aspects

In some aspects, provided herein are anti-MerTK antibodies comprising at least one, two, three, four, five, or six HVRs selected from: (a) HVR-H1 comprising an amino acid sequence selected from the group consisting of SEQ ID NOs: 63, 64, 65, 66, 67, 68, 69, 70, 71, 72, 73, 74, 75, 76, 77, 78, 79, 80, 213, 214, and 224; (b) HVR-H2 comprising an amino acid sequence selected from the group consisting of SEQ ID NOs: 81, 82, 83, 84, 85, 86, 87, 88, 89, 90, 91, 92, 93, 94, 95, 96, 97, 98, 99, 100, 101, 102, 103, 104, 105, 106, 107, 108, 109, 215, 216, and 225; (c) HVR-H3 comprising an amino acid sequence selected from the group consisting of SEQ ID NOs: 110, 111, 112, 113, 114, 115, 116, 117, 118, 119, 120, 121, 122, 123, 124, 125, 126, 127, 128, 129, 130, 131, 132, 133, 134, 135, 136, 217, 218, and 226; (d) HVR-L1 comprising an amino acid sequence selected from the group consisting of SEQ ID NOs: 137, 138, 139, 140, 141, 142, 143, 144, 145, 146, 147, 148, 149, 150, 151, 152, 153, 154, 155, 156, 157, 158, 159, 160, 161, 162, and 220; (e) HVR-L2 comprising an amino acid sequence selected from the group consisting of SEQ ID NOs: 163, 164, 165, 166, 167, 168, 169, 170, 171, 172, 173, 174, 175, 176, 177, 178, 179, 180, 181, 182, 183, 184, and 227; and (f) HVR-L3 comprising an amino acid sequence selected from the group consisting of SEQ ID NOs: 185, 186, 187, 188, 189, 190, 191, 192, 193, 194, 195, 196, 197, 198, 199, 200, 201, 202, 203, 204, 205, 206, 207, 208, 219, 221, and 228.

In some aspects, provided herein are anti-MerTK antibodies comprising at least one, at least two, or all three V_{H} HVR sequences selected from (a) HVR-H1 comprising an amino acid sequence selected from the group consisting of SEQ ID NOs: 63, 64, 65, 66, 67, 68, 69, 70, 71, 72, 73, 74, 75, 76, 77, 78, 79, 80, 213, 214, and 224; (b) HVR-H2 comprising an amino acid sequence selected from the group consisting of SEQ ID NOs: 81, 82, 83, 84, 85, 86, 87, 88, 89, 90, 91, 92, 93, 94, 95, 96, 97, 98, 99, 100, 101, 102, 103, 104, 105, 106, 107, 108, 109, 215, 216, and 225; and (c) HVR-H3 comprising an amino acid sequence selected from the group consisting of SEQ ID NOs: 110, 111, 112, 113, 114, 115, 116, 117, 118, 119, 120, 121, 122, 123, 124, 125, 126, 127, 128, 129, 130, 131, 132, 133, 134, 135, 136, 217, 218, and 226.

In some aspects, provided herein are anti-MerTK antibodies comprising at least one, at least two, or all three V_{L} HVR sequences selected from (a) HVR-L1 comprising an amino acid sequence selected from the group consisting of SEQ ID NOs: 137, 138, 139, 140, 141, 142, 143, 144, 145, 146, 147, 148, 149, 150, 151, 152, 153, 154, 155, 156, 157, 158, 159, 160, 161, 162, and 220; (b) HVR-L2 comprising an amino acid sequence selected from the group consisting of SEQ ID NOs: 163, 164, 165, 166, 167, 168, 169, 170, 171, 172, 173, 174, 175, 176, 177, 178, 179, 180, 181, 182, 183, 184, and 227; and (c) HVR-L3 comprising an amino acid sequence selected from the group consisting of SEQ ID NOs: 185, 186, 187, 188, 189, 190, 191, 192, 193, 194, 195, 196, 197, 198, 199, 200, 201, 202, 203, 204, 205, 206, 207, 208, 219, 221, and 228.

In some aspects, provided herein are anti-MerTK antibodies comprising (a) a V_{H} domain comprising at least one, at least two, or all three V_{H} HVR sequences selected from (i) HVR-H1 comprising an amino acid sequence selected from the group consisting of SEQ ID NOs: 63, 64, 65, 66, 67, 68, 69, 70, 71, 72, 73, 74, 75, 76, 77, 78, 79, 80, 213, 214, and 224, (ii) HVR-H2 comprising an amino acid sequence selected from the group consisting of SEQ ID NOs: 81, 82, 83, 84, 85, 86, 87, 88, 89, 90, 91, 92, 93, 94, 95, 96, 97, 98, 99, 100, 101, 102, 103, 104, 105, 106, 107, 108, 109, 215, 216, and 225, and (iii) HVR-H3 comprising an amino acid sequence selected from the group consisting of SEQ ID NOs: 110, 111, 112, 113, 114, 115, 116, 117, 118, 119, 120, 121, 122, 123, 124, 125, 126, 127, 128, 129, 130, 131, 132, 133, 134, 135, 136, 217, 218, and 226, and (b) a V_{L} domain comprising at least one, at least two, or all three V_{L} HVR sequences selected from (i) HVR-L1 comprising an amino acid sequence selected from the group consisting of SEQ ID NOs: 137, 138, 139, 140, 141, 142, 143, 144, 145, 146, 147, 148, 149, 150, 151, 152, 153, 154, 155, 156, 157, 158, 159, 160, 161, 162, and 220, (ii) HVR-L2 comprising an amino acid sequence selected from the group consisting of SEQ ID NOs: 163, 164, 165, 166, 167, 168, 169, 170, 171, 172, 173, 174, 175, 176, 177, 178, 179, 180, 181, 182, 183, 184, and 227, and (iii) HVR-L3 comprising an amino acid sequence selected from the group consisting of SEQ ID NOs: 185, 186, 187, 188, 189, 190, 191, 192, 193, 194, 195, 196, 197, 198, 199, 200, 201, 202, 203, 204, 205, 206, 207, 208, 219, 221, and 228.

In some aspects, provided herein are anti-MerTK antibodies comprising: (a) HVR-H1 comprising the amino acid sequence of SEQ ID NO:63; (b) HVR-H2 comprising the amino acid sequence of SEQ ID NO:81; (c) HVR-H3 comprising the amino acid sequence of SEQ ID NO:110; (d) HVR-L1 comprising the amino acid sequence of SEQ ID NO:137; (e) HVR-L2 comprising the amino acid sequence of SEQ ID NO:163; and (f) HVR-L3 comprising the amino acid sequence of SEQ ID NO:185; (a) HVR-H1 comprising the amino acid sequence of SEQ ID NO:64; (b) HVR-H2 comprising the amino acid sequence of SEQ ID NO:82; (c) HVR-H3 comprising the amino acid sequence of SEQ ID NO:111; (d) HVR-L1 comprising the amino acid sequence of SEQ ID NO:138; (e) HVR-L2 comprising the amino acid sequence of SEQ ID NO:164; and (f) HVR-L3 comprising the amino acid sequence of SEQ ID NO:186; (a) HVR-H1 comprising the amino acid sequence of SEQ ID NO:65; (b) HVR-H2 comprising the amino acid sequence of SEQ ID NO:83; (c) HVR-H3 comprising the amino acid sequence of SEQ ID NO:112; (d) HVR-L1 comprising the amino acid sequence of SEQ ID NO:139; (e) HVR-L2 comprising the amino acid sequence of SEQ ID NO:165; and (f) HVR-L3 comprising the amino acid sequence of SEQ ID NO:187; (a) HVR-H1 comprising the amino acid sequence of SEQ ID NO:66; (b) HVR-H2 comprising the amino acid sequence of SEQ ID NO:84; (c) HVR-H3 comprising the amino acid sequence of SEQ ID NO:113; (d) HVR-L1 comprising the amino acid sequence of SEQ ID NO:138; (e) HVR-L2 comprising the amino acid sequence of SEQ ID NO:164; and (f) HVR-L3 comprising the amino acid sequence of SEQ ID NO:188; (a) HVR-H1 comprising the amino acid sequence of SEQ ID NO:67; (b) HVR-H2 comprising the amino acid sequence of SEQ ID NO:85; (c) HVR-H3 comprising the amino acid sequence of SEQ ID NO:114; (d) HVR-L1 comprising the amino acid sequence of SEQ ID NO:140; (e) HVR-L2 comprising the amino acid sequence of SEQ ID NO:166; and (f) HVR-L3 comprising the amino acid sequence of SEQ ID NO:189; (a) HVR-H1 comprising the amino acid sequence of SEQ ID NO:68; (b) HVR-H2 comprising the amino acid sequence of SEQ ID NO:86; (c) HVR-H3 comprising the amino acid sequence of SEQ ID NO:115; (d) HVR-L1 comprising the amino acid sequence of SEQ ID NO:141; (e) HVR-L2 comprising the amino acid sequence of SEQ ID NO:167; and (f) HVR-L3 comprising the amino acid sequence of SEQ ID NO:190; (a) HVR-H1 comprising the amino acid sequence of SEQ ID NO:65; (b) HVR-H2 comprising the amino acid sequence of SEQ ID NO:87; (c) HVR-H3 comprising the amino acid sequence of SEQ ID NO:116; (d) HVR-L1 comprising the amino acid sequence of SEQ ID NO:142; (e) HVR-L2 comprising the amino acid sequence of SEQ ID NO:168; and (f) HVR-L3 comprising the amino acid sequence of SEQ ID NO:191; (a) HVR-H1 comprising the amino acid sequence of SEQ ID NO:69; (b) HVR-H2 comprising the amino acid sequence of SEQ ID NO:88; (c) HVR-H3 comprising the amino acid sequence of SEQ ID NO:117; (d) HVR-L1 comprising the amino acid sequence of SEQ ID NO:143; (e) HVR-L2 comprising the amino acid sequence of SEQ ID NO:169; and (f) HVR-L3 comprising the amino acid sequence of SEQ ID NO:192; (a) HVR-H1 comprising the amino acid sequence of SEQ ID NO:70; (b) HVR-H2 comprising the amino acid sequence of SEQ ID NO:89; (c) HVR-H3 comprising the amino acid sequence of SEQ ID NO:118; (d) HVR-L1 comprising the amino acid sequence of SEQ ID NO:144; (e) HVR-L2 comprising the amino acid sequence of SEQ ID NO:163; and (f) HVR-L3 comprising the amino acid sequence of SEQ ID NO:193; (a) HVR-H1 comprising the amino acid sequence of SEQ ID NO:71; (b) HVR-H2 comprising the amino acid sequence of SEQ ID NO:90; (c) HVR-H3 comprising the amino acid sequence of SEQ ID NO:119; (d) HVR-L1 comprising the amino acid sequence of SEQ ID NO:145; (e) HVR-L2 comprising the amino acid sequence of SEQ ID NO:170; and (f) HVR-L3 comprising the amino acid sequence of SEQ ID NO:194; (a) HVR-H1 comprising the amino acid sequence of SEQ ID NO:72; (b) HVR-H2 comprising the amino acid sequence of SEQ ID NO:91; (c) HVR-H3 comprising the amino acid sequence of SEQ ID NO:120; (d) HVR-L1 comprising the amino acid sequence of SEQ ID NO:146; (e) HVR-L2 comprising the amino acid sequence of SEQ ID NO:171; and (f) HVR-L3 comprising the amino acid sequence of SEQ ID NO:195; (a) HVR-H1 comprising the amino acid sequence of SEQ ID NO:73; (b) HVR-H2 comprising the amino acid sequence of SEQ ID NO:92; (c) HVR-H3 comprising the amino acid sequence of SEQ ID NO:121; (d) HVR-L1 comprising the amino acid sequence of SEQ ID NO:147; (e) HVR-L2 comprising the amino acid sequence of SEQ ID NO:172; and (f) HVR-L3 comprising the amino acid sequence of SEQ ID NO:196; (a) HVR-H1 comprising the amino acid sequence of SEQ ID NO:65; (b) HVR-H2 comprising the amino acid sequence of SEQ ID NO:93; (c) HVR-H3 comprising the amino acid sequence of SEQ ID NO:122; (d) HVR-L1 comprising the amino acid sequence of SEQ ID NO:148; (e) HVR-L2 comprising the amino acid sequence of SEQ ID NO:173; and (f) HVR-L3 comprising the amino acid sequence of SEQ ID NO:197; (a) HVR-H1 comprising the amino acid sequence of SEQ ID NO:66; (b) HVR-H2 comprising the amino acid sequence of SEQ ID NO:94; (c) HVR-H3 comprising the amino acid sequence of SEQ ID NO:123; (d) HVR-L1 comprising the amino acid sequence of SEQ ID NO:149; (e) HVR-L2 comprising the amino acid sequence of SEQ ID NO:174; and (f) HVR-L3 comprising the amino acid sequence of SEQ ID NO:198; (a) HVR-H1 comprising the amino acid sequence of SEQ ID NO:66; (b) HVR-H2 comprising the amino acid sequence of SEQ ID NO:95; (c) HVR-H3 comprising the amino acid sequence of SEQ ID NO:124; (d) HVR-L1 comprising the amino acid sequence of SEQ ID NO:150; (e) HVR-L2 comprising the amino acid sequence of SEQ ID NO:164; and (f) HVR-L3 comprising the amino acid sequence of SEQ ID NO:188; (a) HVR-H1 comprising the amino acid sequence of SEQ ID NO:73; (b) HVR-H2 comprising the amino acid sequence of SEQ ID NO:96; (c) HVR-H3 comprising the amino acid sequence of SEQ ID NO:125; (d) HVR-L1 comprising the amino acid sequence of SEQ ID NO:151; (e) HVR-L2 comprising the amino acid sequence of SEQ ID NO:175; and (f) HVR-L3 comprising the amino acid sequence of SEQ ID NO:199; (a) HVR-H1 comprising the amino acid sequence of SEQ ID NO:74; (b) HVR-H2 comprising the amino acid sequence of SEQ ID NO:97; (c) HVR-H3 comprising the amino acid sequence of SEQ ID NO:126; (d) HVR-L1 comprising the amino acid sequence of SEQ ID NO:152; (e) HVR-L2 comprising the amino acid sequence of SEQ ID NO:176; and (f) HVR-L3 comprising the amino acid sequence of SEQ ID NO:200; (a) HVR-H1 comprising the amino acid sequence of SEQ ID NO:71; (b) HVR-H2 comprising the amino acid sequence of SEQ ID NO:98; (c) HVR-H3 comprising the amino acid sequence of SEQ ID NO:127; (d) HVR-L1 comprising the amino acid sequence of SEQ ID NO:153; (e) HVR-L2 comprising the amino acid sequence of SEQ ID NO:177; and (f) HVR-L3 comprising the amino acid sequence of SEQ ID NO:201; (a) HVR-H1 comprising the amino acid sequence of SEQ ID NO:66; (b) HVR-H2 comprising the amino acid sequence of SEQ ID NO:99; (c) HVR-H3 comprising the amino acid sequence of SEQ ID NO:128; (d) HVR-L1 comprising the amino acid sequence of SEQ ID NO:138; (e) HVR-L2 comprising the amino acid sequence of SEQ ID NO:164; and (f) HVR-L3 comprising the amino acid sequence of SEQ ID NO:188; (a) HVR-H1 comprising the amino acid sequence of SEQ ID NO:75; (b) HVR-H2 comprising the amino acid sequence of SEQ ID NO:100; (c) HVR-H3 comprising the amino acid sequence of SEQ ID NO:129; (d) HVR-L1 comprising the amino acid sequence of SEQ ID NO:154; (e) HVR-L2 comprising the amino acid sequence of SEQ ID NO:178; and (f) HVR-L3 comprising the amino acid sequence of SEQ ID NO:202; (a) HVR-H1 comprising the amino acid sequence of SEQ ID NO:71; (b) HVR-H2 comprising the amino acid sequence of SEQ ID NO:101; (c) HVR-H3 comprising the amino acid sequence of SEQ ID NO:130; (d) HVR-L1 comprising the amino acid sequence of SEQ ID NO:155; (e) HVR-L2 comprising the amino acid sequence of SEQ ID NO:179; and (f) HVR-L3 comprising the amino acid sequence of SEQ ID NO:201; (a) HVR-H1 comprising the amino acid sequence of SEQ ID NO:76; (b) HVR-H2 comprising the amino acid sequence of SEQ ID NO:102; (c) HVR-H3 comprising the amino acid sequence of SEQ ID NO:131; (d) HVR-L1 comprising the amino acid sequence of SEQ ID NO:156; (e) HVR-L2 comprising the amino acid sequence of SEQ ID NO:180; and (f) HVR-L3 comprising the amino acid sequence of SEQ ID NO:203; (a) HVR-H1 comprising the amino acid sequence of SEQ ID NO:77; (b) HVR-H2 comprising the amino acid sequence of SEQ ID NO:103; (c) HVR-H3 comprising the amino acid sequence of SEQ ID NO:132; (d) HVR-L1 comprising the amino acid sequence of SEQ ID NO:157; (e) HVR-L2 comprising the amino acid sequence of SEQ ID NO:181; and (f) HVR-L3 comprising the amino acid sequence of SEQ ID NO:204; (a) HVR-H1 comprising the amino acid sequence of SEQ ID NO:78; (b) HVR-H2 comprising the amino acid sequence of SEQ ID NO:104; (c) HVR-H3 comprising the amino acid sequence of SEQ ID NO:133; (d) HVR-L1 comprising the amino acid sequence of SEQ ID NO:158; (e) HVR-L2 comprising the amino acid sequence of SEQ ID NO:182; and (f) HVR-L3 comprising the amino acid sequence of SEQ ID NO:205; (a) HVR-H1 comprising the amino acid sequence of SEQ ID NO:74; (b) HVR-H2 comprising the amino acid sequence of SEQ ID NO:105; (c) HVR-H3 comprising the amino acid sequence of SEQ ID NO:126; (d) HVR-L1 comprising the amino acid sequence of SEQ ID NO:159; (e) HVR-L2 comprising the amino acid sequence of SEQ ID NO:176; and (f) HVR-L3 comprising the amino acid sequence of SEQ ID NO:200; (a) HVR-H1 comprising the amino acid sequence of SEQ ID NO:79; (b) HVR-H2 comprising the amino acid sequence of SEQ ID NO:106; (c) HVR-H3 comprising the amino acid sequence of SEQ ID NO:134; (d) HVR-L1 comprising the amino acid sequence of SEQ ID NO:160; (e) HVR-L2 comprising the amino acid sequence of SEQ ID NO:183; and (f) HVR-L3 comprising the amino acid sequence of SEQ ID NO:206; (a) HVR-H1 comprising the amino acid sequence of SEQ ID NO:74; (b) HVR-H2 comprising the amino acid sequence of SEQ ID NO:107; (c) HVR-H3 comprising the amino acid sequence of SEQ ID NO:126; (d) HVR-L1 comprising the amino acid sequence of SEQ ID NO:161; (e) HVR-L2 comprising the amino acid sequence of SEQ ID NO:176; and (f) HVR-L3 comprising the amino acid sequence of SEQ ID NO:200; (a) HVR-H1 comprising the amino acid sequence of SEQ ID NO:70; (b) HVR-H2 comprising the amino acid sequence of SEQ ID NO:108; (c) HVR-H3 comprising the amino acid sequence of SEQ ID NO:135; (d) HVR-L1 comprising the amino acid sequence of SEQ ID NO: 144; (e) HVR-L2 comprising the amino acid sequence of SEQ ID NO:170; and (f) HVR-L3 comprising the amino acid sequence of SEQ ID NO:207; (a) HVR-H1 comprising the amino acid sequence of SEQ ID NO:80; (b) HVR-H2 comprising the amino acid sequence of SEQ ID NO:109; (c) HVR-H3 comprising the amino acid sequence of SEQ ID NO:136; (d) HVR-L1 comprising the amino acid sequence of SEQ ID NO:162; (e) HVR-L2 comprising the amino acid sequence of SEQ ID NO:184; and (f) HVR-L3 comprising the amino acid sequence of SEQ ID NO:208; (a) HVR-H1 comprising the amino acid sequence of SEQ ID NO:213; (b) HVR-H2 comprising the amino acid sequence of SEQ ID NO:215; (c) HVR-H3 comprising the amino acid sequence of SEQ ID NO:217; (d) HVR-L1 comprising the amino acid sequence of SEQ ID NO:156; (e) HVR-L2 comprising the amino acid sequence of SEQ ID NO:180; and (f) HVR-L3 comprising the amino acid sequence of SEQ ID NO:219; (a) HVR-H1 comprising the amino acid sequence of SEQ ID NO:214; (b) HVR-H2 comprising the amino acid sequence of SEQ ID NO:216; (c) HVR-H3 comprising the amino acid sequence of SEQ ID NO:218; (d) HVR-L1 comprising the amino acid sequence of SEQ ID NO:220; (e) HVR-L2 comprising the amino acid sequence of SEQ ID NO:172; and (f) HVR-L3 comprising the amino acid sequence of SEQ ID NO:221; (a) HVR-H1 comprising the amino acid sequence of SEQ ID NO:224; (b) HVR-H2 comprising the amino acid sequence of SEQ ID NO:225; (c) HVR-H3 comprising the amino acid sequence of SEQ ID NO:226; (d) HVR-L1 comprising the amino acid sequence of SEQ ID NO:146; (e) HVR-L2 comprising the amino acid sequence of SEQ ID NO:227; and (f) HVR-L3 comprising the amino acid sequence of SEQ ID NO:228.

In another aspect, an anti-MerTK antibody comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs: 5, 6, 7, 8, 9, 10, 11, 12, 13, 14, 15, 16, 17, 18, 19, 20, 21, 22, 23, 24, 25, 26, 27, 28, 29, 30, 31, 32, 33, 209, 210, and 222. In certain aspects, a V_{H} sequence having at least 90%, 91%, 92%, 93%, 94%, 95%, 96%, 97%, 98%, or 99% identity to an amino acid sequence selected from the group consisting of SEQ ID NOs: 5, 6, 7, 8, 9, 10, 11, 12, 13, 14, 15, 16, 17, 18, 19, 20, 21, 22, 23, 24, 25, 26, 27, 28, 29, 30, 31, 32, 33, 209, 210, and 222 contains substitutions (e.g., conservative substitutions), insertions, or deletions relative to the reference sequence, but an anti-MerTK antibody comprising that sequence retains the ability to bind to MerTK. In certain aspects, a total of 1 to 10 amino acids have been substituted, inserted, and/or deleted in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 209, 210, or 222. In certain aspects, a total of 1 to 5 amino acids have been substituted, inserted and/or deleted in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 209, 210, or 222. In certain aspects, substitutions, insertions, or deletions occur in regions outside the HVRs (i.e., in the FRs). Optionally, the anti-MerTK antibody comprises the V_{H} sequence of SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 209, 210, or 222, including post-translational modifications of that sequence. In a particular aspect, the V_{H} comprises one, two or three HVRs selected from: (a) HVR-H1 comprising an amino acid sequence selected from the group consisting of SEQ ID NOs:63-80, 213, 214, and 224 (b) HVR-H2 comprising an amino acid sequence selected from the group consisting of SEQ ID NOs:81-109, 215, 216, and 225 and (c) HVR-H3 comprising an amino acid sequence selected from the group consisting of SEQ ID NOs:110-136, 217, 218, and 226.

In another aspect, an anti-MerTK antibody is provided, wherein the antibody comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs: 34, 35, 36, 37, 38, 39, 40, 41, 42, 43, 44, 45, 46, 47, 48, 49, 50, 51, 52, 53, 54, 55, 56, 57, 58, 59, 60, 61, 62, 211, 212, and 223. In certain aspects, a V_{L} sequence having at least 90%, 91%, 92%, 93%, 94%, 95%, 96%, 97%, 98%, or 99% identity to an amino acid sequence selected from the group consisting of SEQ ID NOs: 34, 35, 36, 37, 38, 39, 40, 41, 42, 43, 44, 45, 46, 47, 48, 49, 50, 51, 52, 53, 54, 55, 56, 57, 58, 59, 60, 61, 62, 211, 212, and 223, and contains substitutions (e.g., conservative substitutions), insertions, or deletions relative to the reference sequence, but an anti-MerTK antibody comprising that sequence retains the ability to bind to MerTK. In some aspects, a total of 1 to 10 amino acids have been substituted, inserted and/or deleted in SEQ ID NO: 34, 35, 36, 37, 38, 39, 40, 41, 42, 43, 44, 45, 46, 47, 48, 49, 50, 51, 52, 53, 54, 55, 56, 57, 58, 59, 60, 61, 62, 211, 212, or 223. In certain aspects, a total of 1 to 5 amino acids have been substituted, inserted and/or deleted in SEQ ID NO: 34, 35, 36, 37, 38, 39, 40, 41, 42, 43, 44, 45, 46, 47, 48, 49, 50, 51, 52, 53, 54, 55, 56, 57, 58, 59, 60, 61, 62, 211, 212, or 223. In certain aspects, the substitutions, insertions, or deletions occur in regions outside the HVRs (i.e., in the FRs). Optionally, the anti-MerTK antibody comprises the V_{L} sequence of SEQ ID NO: 34, 35, 36, 37, 38, 39, 40, 41, 42, 43, 44, 45, 46, 47, 48, 49, 50, 51, 52, 53, 54, 55, 56, 57, 58, 59, 60, 61, 62, 211, 212, or 223, including post-translational modifications of that sequence. In a particular aspect, the V_{L} comprises one, two or three HVRs selected from (a) HVR-L1 comprising an amino acid sequence selected from the group consisting of SEQ ID NOs:137-162, and 220, (b) HVR-L2 comprising an amino acid sequence selected from the group consisting of SEQ ID NOs: 163-184, and 227, and (c) HVR-L3 comprising an amino acid sequence selected from the group consisting of SEQ ID NOs: 185-208, 219, 221, and 228.

In some aspects, an anti-MerTK antibody is provided, wherein the antibody comprises a V_{H} as in any of the aspects provided above, and a V_{L} as in any of the aspects provided above. In some aspects, provided herein are anti-MerTK antibodies, wherein the antibody comprises a V_{H} as in any of the aspects provided above, and a V_{L} as in any of the aspects provided above. In one aspect, the antibody comprises the V_{H} and V_{L} sequences in SEQ ID NOs:5-33, 209, 210, and 222 and SEQ ID NOs:34-62, 211, 212, and 223, respectively, including post-translational modifications of those sequences.

In some aspects, provided herein are anti-MerTK antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO:5 and V_{L} comprising the amino acid sequence of SEQ ID NO:34; V_{H} comprising the amino acid sequence of SEQ ID NO:6 and V_{L} comprising the amino acid sequence of SEQ ID NO:35; V_{H} comprising the amino acid sequence of SEQ ID NO:7 and V_{L} comprising the amino acid sequence of SEQ ID NO:36; V_{H} comprising the amino acid sequence of SEQ ID NO:8 and V_{L} comprising the amino acid sequence of SEQ ID NO:37; V_{H} comprising the amino acid sequence of SEQ ID NO:9 and V_{L} comprising the amino acid sequence of SEQ ID NO:38; V_{H} comprising the amino acid sequence of SEQ ID NO:10 and V_{L} comprising the amino acid sequence of SEQ ID NO:39; V_{H} comprising the amino acid sequence of SEQ ID NO:11 and V_{L} comprising the amino acid sequence of SEQ ID NO:40; V_{H} comprising the amino acid sequence of SEQ ID NO:12 and V_{L} comprising the amino acid sequence of SEQ ID NO:41; V_{H} comprising the amino acid sequence of SEQ ID NO:13 and V_{L} comprising the amino acid sequence of SEQ ID NO:42; V_{H} comprising the amino acid sequence of SEQ ID NO:14 and V_{L} comprising the amino acid sequence of SEQ ID NO:43; V_{H} comprising the amino acid sequence of SEQ ID NO:15 and V_{L} comprising the amino acid sequence of SEQ ID NO:44; V_{H} comprising the amino acid sequence of SEQ ID NO:16 and V_{L} comprising the amino acid sequence of SEQ ID NO:45; V_{H} comprising the amino acid sequence of SEQ ID NO:17 and V_{L} comprising the amino acid sequence of SEQ ID NO:46; V_{H} comprising the amino acid sequence of SEQ ID NO:18 and V_{L} comprising the amino acid sequence of SEQ ID NO:47; V_{H} comprising the amino acid sequence of SEQ ID NO:19 and V_{L} comprising the amino acid sequence of SEQ ID NO:48; V_{H} comprising the amino acid sequence of SEQ ID NO:20 and V_{L} comprising the amino acid sequence of SEQ ID NO:49; V_{H} comprising the amino acid sequence of SEQ ID NO:21 and V_{L} comprising the amino acid sequence of SEQ ID NO:50; V_{H} comprising the amino acid sequence of SEQ ID NO:22 and V_{L} comprising the amino acid sequence of SEQ ID NO:51; V_{H} comprising the amino acid sequence of SEQ ID NO:23 and V_{L} comprising the amino acid sequence of SEQ ID NO:52; V_{H} comprising the amino acid sequence of SEQ ID NO:24 and V_{L} comprising the amino acid sequence of SEQ ID NO:53; V_{H} comprising the amino acid sequence of SEQ ID NO:25 and V_{L} comprising the amino acid sequence of SEQ ID NO:54; V_{H} comprising the amino acid sequence of SEQ ID NO:26 and V_{L} comprising the amino acid sequence of SEQ ID NO:55; V_{H} comprising the amino acid sequence of SEQ ID NO:27 and V_{L} comprising the amino acid sequence of SEQ ID NO:56; V_{H} comprising the amino acid sequence of SEQ ID NO:28 and V_{L} comprising the amino acid sequence of SEQ ID NO:57; V_{H} comprising the amino acid sequence of SEQ ID NO:29 and V_{L} comprising the amino acid sequence of SEQ ID NO:58; V_{H} comprising the amino acid sequence of SEQ ID NO:30 and V_{L} comprising the amino acid sequence of SEQ ID NO:59; V_{H} comprising the amino acid sequence of SEQ ID NO:31 and V_{L} comprising the amino acid sequence of SEQ ID NO:60; V_{H} comprising the amino acid sequence of SEQ ID NO:32 and V_{L} comprising the amino acid sequence of SEQ ID NO:61; V_{H} comprising the amino acid sequence of SEQ ID NO:33 and V_{L} comprising the amino acid sequence of SEQ ID NO:62; V_{H} comprising the amino acid sequence of SEQ ID NO:209 and V_{L} comprising the amino acid sequence of SEQ ID NO:211; V_{H} comprising the amino acid sequence of SEQ ID NO:210 and V_{L} comprising the amino acid sequence of SEQ ID NO:212; and V_{H} comprising the amino acid sequence of SEQ ID NO:222 and V_{L} comprising the amino acid sequence of SEQ ID NO:223.

In some aspects, an anti-MerTK antibody of the present disclosure competitively inhibits binding of at least one reference antibody selected from MTK-201, MTK-202, MTK-203, MTK-204, MTK-205, MTK-206, MTK-207, MTK-208, MTK-209, MTK-210, MTK-211, MTK-212, MTK-213, MTK-214, MTK-215, MTK-216, MTK-217, MTK-218, MTK-219, MTK-220, MTK-221, MTK-222, MTK-223, MTK-224, MTK-225, MTK-226, MTK-227, MTK-228, MTK-229, MTK-230, MTK-231, and MTK-232 and any combination thereof, for binding to MerTK.

In some aspects, an anti-MerTK antibody of the present disclosure binds to an epitope of human MerTK that is the same as or overlaps with the MerTK epitope bound by at least one reference antibody selected from MTK-201, MTK-202, MTK-203, MTK-204, MTK-205, MTK-206, MTK-207, MTK-208, MTK-209, MTK-210, MTK-211, MTK-212, MTK-213, MTK-214, MTK-215, MTK-216, MTK-217, MTK-218, MTK-219, MTK-220, MTK-221, MTK-222, MTK-223, MTK-224, MTK-225, MTK-226, MTK-227, MTK-228, MTK-229, MTK-230, MTK-231, and MTK-232. Detailed exemplary methods for mapping an epitope to which an antibody binds are provided in Morris (1996) “Epitope Mapping Protocols,” in Methods in Molecular Biology vol. 66 (Humana Press, Totowa, NJ).

In some aspects, an anti-MerTK antibody of the present disclosure competitively inhibits binding of at least one reference antibody, or binds to an epitope of human MerTK that is the same as or overlaps with the MerTK epitope bound by at least one reference antibody, wherein the reference antibody is an anti-MerTK antibody comprising a heavy chain variable domain (V_{H}) and a light chain variable domain (V_{L}), wherein the V_{H} and V_{L} are selected from the group consisting of: V_{H} comprising the amino acid sequence of SEQ ID NO:5 and V_{L} comprising the amino acid sequence of SEQ ID NO:34; V_{H} comprising the amino acid sequence of SEQ ID NO:6 and V_{L} comprising the amino acid sequence of SEQ ID NO:35; V_{H} comprising the amino acid sequence of SEQ ID NO:7 and V_{L} comprising the amino acid sequence of SEQ ID NO:36; V_{H} comprising the amino acid sequence of SEQ ID NO:8 and V_{L} comprising the amino acid sequence of SEQ ID NO:37; V_{H} comprising the amino acid sequence of SEQ ID NO:9 and V_{L} comprising the amino acid sequence of SEQ ID NO:38; V_{H} comprising the amino acid sequence of SEQ ID NO:10 and V_{L} comprising the amino acid sequence of SEQ ID NO:39; V_{H} comprising the amino acid sequence of SEQ ID NO:11 and V_{L} comprising the amino acid sequence of SEQ ID NO:40; V_{H} comprising the amino acid sequence of SEQ ID NO:12 and V_{L} comprising the amino acid sequence of SEQ ID NO:41; V_{H} comprising the amino acid sequence of SEQ ID NO:13 and V_{L} comprising the amino acid sequence of SEQ ID NO:42; V_{H} comprising the amino acid sequence of SEQ ID NO:14 and V_{L} comprising the amino acid sequence of SEQ ID NO:43; V_{H} comprising the amino acid sequence of SEQ ID NO:15 and V_{L} comprising the amino acid sequence of SEQ ID NO:44; V_{H} comprising the amino acid sequence of SEQ ID NO:16 and V_{L} comprising the amino acid sequence of SEQ ID NO:45; V_{H} comprising the amino acid sequence of SEQ ID NO:17 and V_{L} comprising the amino acid sequence of SEQ ID NO:46; V_{H} comprising the amino acid sequence of SEQ ID NO:18 and V_{L} comprising the amino acid sequence of SEQ ID NO:47; V_{H} comprising the amino acid sequence of SEQ ID NO:19 and V_{L} comprising the amino acid sequence of SEQ ID NO:48; V_{H} comprising the amino acid sequence of SEQ ID NO:20 and V_{L} comprising the amino acid sequence of SEQ ID NO:49; V_{H} comprising the amino acid sequence of SEQ ID NO:21 and V_{L} comprising the amino acid sequence of SEQ ID NO:50; V_{H} comprising the amino acid sequence of SEQ ID NO:22 and V_{L} comprising the amino acid sequence of SEQ ID NO:51; V_{H} comprising the amino acid sequence of SEQ ID NO:23 and V_{L} comprising the amino acid sequence of SEQ ID NO:52; V_{H} comprising the amino acid sequence of SEQ ID NO:24 and V_{L} comprising the amino acid sequence of SEQ ID NO:53; V_{H} comprising the amino acid sequence of SEQ ID NO:25 and V_{L} comprising the amino acid sequence of SEQ ID NO:54; V_{H} comprising the amino acid sequence of SEQ ID NO:26 and V_{L} comprising the amino acid sequence of SEQ ID NO:55; V_{H} comprising the amino acid sequence of SEQ ID NO:27 and V_{L} comprising the amino acid sequence of SEQ ID NO:56; V_{H} comprising the amino acid sequence of SEQ ID NO:28 and V_{L} comprising the amino acid sequence of SEQ ID NO:57; V_{H} comprising the amino acid sequence of SEQ ID NO:29 and V_{L} comprising the amino acid sequence of SEQ ID NO:58; V_{H} comprising the amino acid sequence of SEQ ID NO:30 and V_{L} comprising the amino acid sequence of SEQ ID NO:59; V_{H} comprising the amino acid sequence of SEQ ID NO:31 and V_{L} comprising the amino acid sequence of SEQ ID NO:60; V_{H} comprising the amino acid sequence of SEQ ID NO:32 and V_{L} comprising the amino acid sequence of SEQ ID NO:61; V_{H} comprising the amino acid sequence of SEQ ID NO:33 and V_{L} comprising the amino acid sequence of SEQ ID NO:62; V_{H} comprising the amino acid sequence of SEQ ID NO:209 and V_{L} comprising the amino acid sequence of SEQ ID NO:211; V_{H} comprising the amino acid sequence of SEQ ID NO:210 and V_{L} comprising the amino acid sequence of SEQ ID NO:212; and V_{H} comprising the amino acid sequence of SEQ ID NO:222 and V_{L} comprising the amino acid sequence of SEQ ID NO:223.

In some aspects, the anti-MerTK antibody according to any of the above aspects is a monoclonal antibody, including a humanized and/or human antibody. In some aspects, the anti-MerTK antibody is an antibody fragment, e.g., a Fv, Fab, Fab′, scFv, diabody, or F(ab′)2 fragment. In some aspects, the anti-MerTK antibody is a substantially full-length antibody, e.g., an IgG1 antibody, IgG2a antibody or other antibody class or isotype as defined herein.

In some aspects, an anti-MerTK antibody according to any of the above aspects may incorporate any of the features, singly or in combination, as described in Sections 1-7 below:

### (1) Anti-MerTK Antibody Binding Affinity

In some aspects of any of the antibodies provided herein, the antibody has a dissociation constant (K_{D}) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M). Dissociation constants may be determined through any analytical technique, including any biochemical or biophysical technique such as ELISA, surface plasmon resonance (SPR), bio-layer interferometry (see, e.g., Octet System by ForteBio), isothermal titration calorimetry (ITC), differential scanning calorimetry (DSC), circular dichroism (CD), stopped-flow analysis, and colorimetric or fluorescent protein melting analyses. In one aspect, Kd is measured by a radiolabeled antigen binding assay (RIA). In some aspect, an RIA is performed with the Fab version of an antibody of interest and its antigen, for example as described in Chen et al. J. Mol. Biol. 293:865-881(1999)). In some aspects, K_{D} is measured using a BIACORE surface plasmon resonance assay, for example, an assay using a BIACORE-2000 or a BIACORE-3000 (BIAcore, Inc., Piscataway, NJ) is performed at 25° C. with immobilized antigen CM5 chips at ˜10 response units (RU). In some aspects, the K_{D} is determined using a monovalent antibody (e.g., a Fab) or a full-length antibody. In some aspects, the K_{D} is determined using a full-length antibody in a monovalent form.

In some aspects, an anti-MerTK antibody of the present disclosure binds to human MerTK, wherein the K_{D} of binding to human MerTK is from about 1.4 nM to about 81 nM. In some aspects, an anti-MerTK antibody binds to cyno MerTK, wherein the K_{D} of binding to cyno MerTK is from about 1.6 nM to about 107 nM. In some aspects, an anti-MerTK antibody of the present disclosure binds to murine MerTK, wherein the K_{D} of binding to murine MerTK is from about 30 nM to about 186 nM.

### (2) Antibody Fragments

In some aspects of any of the antibodies provided herein, the antibody is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In certain aspects, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### (3) Chimeric and Humanized Antibodies

In some aspects of any of the antibodies provided herein, the antibody is a chimeric antibody. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some aspects of any of the antibodies provided herein, the antibody is a humanized antibody. Typically, a non-human antibody is humanized to reduce immunogenicity to humans, while retaining the specificity and affinity of the parental non-human antibody. In certain aspects, a humanized antibody is substantially non-immunogenic in humans. In certain aspects, a humanized antibody has substantially the same affinity for a target as an antibody from another species from which the humanized antibody is derived. See, e.g., U.S. Pat. Nos. 5,530,101, 5,693,761; 5,693,762; and 5,585,089. In certain aspects, amino acids of an antibody variable domain that can be modified without diminishing the native affinity of the antigen binding domain while reducing its immunogenicity are identified. See, e.g., U.S. Pat. Nos. 5,766,886 and 5,869,619. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

### (4) Human Antibodies

In some aspects of any of the antibodies provided herein, the antibody is a human antibody. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human MAbs with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al. J. Immunol. 147:86 (1991)). Human antibodies generated via human B-cell hybridoma technology are also described in Li et al. Proc. Natl. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20(3):927-937 (2005) and Vollmers et al. Methods and Findings in Experimental and Clinical Pharmacology 27(3):185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

In some aspects of any of the antibodies provided herein, the antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast display (Adimab), and the like. In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol. 12: 433-455 (1994). For example, a variety of methods are known in the art for generating phage display libraries and screening such libraries for antibodies possessing the desired binding characteristics. See also Sidhu et al. J. Mol. Biol. 338(2): 299-310, 2004; Lee et al. J. Mol. Biol. 340(5): 1073-1093, 2004; Fellouse Proc. Natl. Acad. Sci. USA 101(34):12467-12472 (2004); and Lee et al. J. Immunol. Methods 284(-2):1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J 12: 725-734 (1993). Finally, naive libraries can also be made synthetically by cloning unrearranged V-gene segments from stem cells, and using PCR primers comprising random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

### (5) Constant Regions Including Fc Regions

In some aspects of any of the antibodies provided herein, the antibody comprises an Fc. In some aspects, the Fe is a human IgG1, IgG2, IgG3, and/or IgG4 isotype. In some aspects, the antibody is of the IgG class, the IgM class, or the IgA class.

In certain aspects of any of the antibodies provided herein, the antibody has an IgG2 isotype. In some aspects, the antibody contains a human IgG2 constant region. In some aspects, the human IgG2 constant region includes an Fc region. In some aspects, the antibody induces the one or more MerTK activities or independently of binding to an Fc receptor. In some aspects, the antibody binds an inhibitory Fc receptor. In certain aspects, the inhibitory Fc receptor is inhibitory Fc-gamma receptor IIB (FcγIIB).

In certain aspects of any of the antibodies provided herein, the antibody has an IgG1 isotype. In some aspects, the antibody contains a mouse IgG1 constant region. In some aspects, the antibody contains a human IgG1 constant region. In some aspects, the human IgG1 constant region includes an Fc region. In some aspects, the antibody binds an inhibitory Fc receptor. In certain aspects, the inhibitory Fc receptor is inhibitory Fc-gamma receptor IIB (FcγIIB).

In certain aspects of any of the antibodies provided herein, the antibody has an IgG4 isotype. In some aspects, the antibody contains a human IgG4 constant region. In some aspects, the human IgG4 constant region includes an Fc region. In some aspects, the antibody binds an inhibitory Fc receptor. In certain aspects, the inhibitory Fc receptor is inhibitory Fc-gamma receptor IIB (FcγIIB).

In certain aspects of any of the antibodies provided herein, the antibody has a hybrid IgG2/4 isotype. In some aspects, the antibody includes an amino acid sequence comprising amino acids 118 to 260 according to EU numbering of human IgG2 and amino acids 261-447 according to EU numbering of human IgG4 (WO 1997/11971; WO 2007/106585).

In some aspects, the Fc region increases clustering without activating complement as compared to a corresponding antibody comprising an Fc region that does not comprise the amino acid substitutions. In some aspects, the antibody induces one or more activities of a target specifically bound by the antibody. In some aspects, the antibody binds to MerTK.

It may also be desirable to modify an anti-MerTK antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some aspects, the effector function is impaired by removing N-glycosylation of the Fc region (e.g., in the CH2 domain of IgG) of the antibody. In some aspects, the effector function is impaired by modifying regions such as 233-236, 297, and/or 327-331 of human IgG as described in WO 99/58572 and Armour et al. Molecular Immunology 40: 585-593 (2003); Reddy et al. J. Immunology 164:1925-1933 (2000). In other aspects, it may also be desirable to modify an anti-MerTK antibody of the present disclosure to modify effector function to increase finding selectivity toward the ITIM-containing FcgRIIb (CD32b) to increase clustering of MerTK antibodies on adjacent cells without activating humoral responses including Antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Other amino acid sequence modifications.

### (6) Antibody Variants

In some aspects of any of the antibodies provided herein, amino acid sequence variants of the antibodies are contemplated. For example, it may be desirable to improve the binding affinity and/or other biological properties of the antibody.

### (i) Substitution, Insertion, and Deletion Variants

In some aspects of any of the antibodies provided herein, antibody variants having one or more amino acid substitutions are provided. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

TABLE A
Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the polypeptide or antibody described herein, according to certain aspects, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al. J. Mol. Biol., 157:105-131 (1982). It is known that certain amino acids can be substituted for other amino acids having a similar hydropathic index or score and still retain a similar biological activity. In making changes based upon the hydropathic index, in certain aspects, the substitution of amino acids whose hydropathic indices are within ±2 is included. In certain aspects, those which are within ±1 are included, and in certain aspects, those within ±0.5 are included.

It is also understood in the art that the substitution of like amino acids can be made effectively on the basis of hydrophilicity, particularly where the biologically functional protein or peptide thereby created is intended for use in immunological aspects, as in the present case. In certain aspects, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain aspects, the substitution of amino acids whose hydrophilicity values are within ±2 is included, in certain aspects, those which are within ±1 are included, and in certain aspects, those within +0.5 are included. One can also identify epitopes from primary amino acid sequences on the basis of hydrophilicity. These regions are also referred to as “epitopic core regions”.

In certain aspects of the variant VH and VL sequences provided above, each HVR is unaltered.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Any cysteine residue outside the HVRs and not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

### (ii) Glycosylation Variants

In some aspects of any of the antibodies provided herein, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn297 according to Kabat numbering of the CH2 domain of the Fc region. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the disclosure may be made in order to create antibody variants with certain improved properties.

In one aspect, antibody variants are provided having a carbohydrate structure that lacks fucose attached (directly or indirectly) to an Fc region. See, e.g., US Patent Publication Nos. 2003/0157108 and 2004/0093621. Examples of publications related to “defucosylated” or “fucose-deficient” antibody variants include: US 2003/0157108; US 2003/0115614; US 2002/0164328; US 2004/0093621; US 2004/0132140; US 2004/0110704; US 2004/0110282; US 2004/0109865; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Led 3 CHO cells deficient in protein fucosylation (Ripka et al. Arch. Biochem. Biophys. 249:533-545 (1986); US 2003/0157108), and knockout cell lines, such as alpha-1,6-fucosyltransferase gene, FUT8, knockout CHO cells (see, e.g., Yamane-Ohnuki et al. Biotech. Bioeng. 87: 614 (2004) and Kanda et al. Biotechnol. Bioeng. 94(4):680-688 (2006)).

(iii) Modified Constant Regions

In some aspects of any of the antibodies provided herein, the antibody Fe is an antibody Fe isotypes and/or modifications. In some aspects, the antibody Fc isotype and/or modification is capable of binding to Fc gamma receptor.

In some aspects of any of the antibodies provided herein, the modified antibody Fc is an IgG1 modified Fc. In some aspects, the IgG1 modified Fc comprises one or more modifications. For example, in some aspects, the IgG1 modified Fc comprises one or more amino acid substitutions (e.g., relative to a wild-type Fc region of the same isotype). In some aspects, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention.

In some aspects of any of the IgG1 modified Fc, the Fc comprises N297A mutation according to EU numbering. In some aspects of any of the IgG1 modified Fc, the Fc comprises D265A and N297A mutations according to EU numbering. In some aspects of any of the IgG1 modified Fc, the Fc comprises D270A mutations according to EU numbering. In some aspects, the IgG1 modified Fc comprises L234A and L235A mutations according to EU numbering. In some aspects of any of the IgG1 modified Fc, the Fc comprises L234A and G237A mutations according to EU numbering. In some aspects of any of the IgG1 modified Fc, the Fc comprises L234A, L235A and G237A mutations according to EU numbering. In some aspects of any of the IgG1 modified Fc, the Fc comprises one or more (including all) of P238D, L328E, E233, G237D, H268D, P271G and A330R mutations according to EU numbering. In some aspects of any of the IgG1 modified Fc, the Fc comprises one or more of S267E/L328F mutations according to EU numbering. In some aspects of any of the IgG1 modified Fc, the Fc comprises P238D, L328E, E233D, G237D, H268D, P271G and A330R mutations according to EU numbering. In some aspects of any of the IgG1 modified Fe, the Fc comprises P238D, L328E, G237D, H268D, P271G and A330R mutations according to EU numbering. In some aspects of any of the IgG1 modified Fc, the Fc comprises P238D, S267E, L328E, E233D, G237D, H268D, P271G and A330R mutations according to EU numbering. In some aspects of any of the IgG1 modified Fc, the Fc comprises P238D, S267E, L328E, G237D, H268D, P271G and A330R mutations according to EU numbering. In some aspects of any of the IgG1 modified Fc, the Fc comprises C226S, C229S, E233P, L234V, and L235A mutations according to EU numbering. In some aspects of any of the IgG1 modified Fc, the Fc comprises L234F, L235E, and P331S mutations according to EU numbering. In some aspects of any of the IgG1 modified Fc, the Fc comprises S267E and L328F mutations according to EU numbering. In some aspects of any of the IgG1 modified Fc, the Fc comprises N325S and L328F mutations according to EU numbering. In some aspects of any of the IgG1 modified Fc, the Fc comprises S267E mutations according to EU numbering. In some aspects of any of the IgG1 modified Fc, the Fc comprises a substitute of the constant heavy 1 (CH1) and hinge region of IgG1 with CH1 and hinge region of IgG2 (amino acids 118-230 of IgG2 according to EU numbering) with a Kappa light chain.

In some aspects of any of the IgG1 modified Fc, the Fc includes two or more amino acid substitutions that increase antibody clustering without activating complement as compared to a corresponding antibody having an Fc region that does not include the two or more amino acid substitutions. Accordingly, in some aspects of any of the IgG1 modified Fc, the IgG1 modified Fc is an antibody comprising an Fc region, where the antibody comprises an amino acid substitution at position E430G and one or more amino acid substitutions in the Fc region at a residue position selected from: L234F, L235A, L235E, S267E, K322A, L328F, A330S, P331S, and any combination thereof according to EU numbering. In some aspects, the IgG1 modified Fc comprises an amino acid substitution at positions E430G, L243A, L235A, and P331S according to EU numbering. In some aspects, the IgG1 modified Fc comprises an amino acid substitution at positions E430G and P331S according to EU numbering. In some aspects, the IgG1 modified Fc comprises an amino acid substitution at positions E430G and K322A according to EU numbering. In some aspects, the IgG1 modified Fc comprises an amino acid substitution at positions E430G, A330S, and P331S according to EU numbering. In some aspects, the IgG1 modified Fc comprises an amino acid substitution at positions E430G, K322A, A330S, and P331S according to EU numbering. In some aspects, the IgG1 modified Fc comprises an amino acid substitution at positions E430G, K322A, and A330S according to EU numbering. In some aspects, the IgG1 modified Fc comprises an amino acid substitution at positions E430G, K322A, and P331S according to EU numbering.

In some aspects of any of the IgG1 modified Fc, the IgG1 modified Fc may further comprise herein may be combined with an A330L mutation (Lazar et al. Proc Natl Acad Sci USA, 103:4005-4010 (2006)), or one or more of L234F, L235E, and/or P331S mutations (Sazinsky et al. Proc Natl Acad Sci USA, 105:20167-20172 (2008)), according to the EU numbering convention, to eliminate complement activation. In some aspects of any of the IgG1 modified Fc, the IgG1 modified Fc may further comprise one or more of A330L, A330S, L234F, L235E, and/or P331S according to EU numbering. In some aspects of any of the IgG1 modified Fc, the IgG1 modified Fc may further comprise one or more mutations to enhance the antibody half-life in human serum (e.g., one or more (including all) of M252Y, S254T, and T256E mutations according to the EU numbering convention). In some aspects of any of the IgG1 modified Fc, the IgG1 modified Fc may further comprise one or more of E430G, E430S, E430F, E430T, E345K, E345Q, E345R, E345Y, S440Y, and/or S440W according to EU numbering.

Other aspects of the present disclosure relate to antibodies having modified constant regions (i.e., Fc regions). An antibody dependent on binding to FcgR receptor to activate targeted receptors may lose its agonist activity if engineered to eliminate FcgR binding (see, e.g., Wilson et al. Cancer Cell 19:101-113 (2011); Armour at al. Immunology 40:585-593 (2003); and White et al. Cancer Cell 27:138-148 (2015)). As such, it is thought that an anti-MerTK antibody of the present disclosure with the correct epitope specificity can activate the target antigen, with minimal adverse effects, when the antibody has an Fc domain from a human IgG2 isotype (CH1 and hinge region) or another type of Fc domain that is capable of preferentially binding the inhibitory FcgRIIB r receptors, or a variation thereof.

In some aspects of any of the antibodies provided herein, the modified antibody Fc is an IgG2 modified Fc. In some aspects, the IgG2 modified Fc comprises one or more modifications. For example, in some aspects, the IgG2 modified Fc comprises one or more amino acid substitutions (e.g., relative to a wild-type Fc region of the same isotype). In some aspects of any of the IgG2 modified Fc, the one or more amino acid substitutions are selected from V234A (Alegre et al. Transplantation 57:1537-1543 (1994); Xu et al. Cell Immunol, 200:16-26 (2000)); G237A (Cole et al. Transplantation, 68:563-571 (1999)); H268Q, V309L, A330S, P331S (US 2007/0148167; Armour et al. Eur J Immunol 29: 2613-2624 (1999); Armour et al. The Haematology Journal 1(Suppl.1):27 (2000); Armour et al. The Haematology Journal 1(Suppl.1):27 (2000)), C219S, and/or C220S (White et al. Cancer Cell 27, 138-148 (2015)); S267E, L328F (Chu et al. Mol Immunol, 45:3926-3933 (2008)); and M252Y, S254T, and/or T256E according to the EU numbering convention. In some aspects of any of the IgG2 modified Fc, the Fc comprises an amino acid substitution at positions V234A and G237A according to EU numbering. In some aspects of any of the IgG2 modified Fc, the Fc comprises an amino acid substitution at positions C219S or C220S according to EU numbering. In some aspects of any of the IgG2 modified Fc, the Fc comprises an amino acid substitution at positions A330S and P331S according to EU numbering. In some aspects of any of the IgG2 modified Fc, the Fc comprises an amino acid substitution at positions S267E and L328F according to EU numbering.

In some aspects of any of the IgG2 modified Fc, the Fc comprises a C127S amino acid substitution according to the EU numbering convention (White et al., (2015) Cancer Cell 27, 138-148; Lightle et al. Protein Sci. 19:753-762 (2010); and WO 2008/079246). In some aspects of any of the IgG2 modified Fc, the antibody has an IgG2 isotype with a Kappa light chain constant domain that comprises a C214S amino acid substitution according to the EU numbering convention (White et al. Cancer Cell 27:138-148 (2015); Lightle et al. Protein Sci. 19:753-762 (2010); and WO 2008/079246).

In some aspects of any of the IgG2 modified Fc, the Fc comprises a C220S amino acid substitution according to the EU numbering convention. In some aspects of any of the IgG2 modified Fc, the antibody has an IgG2 isotype with a Kappa light chain constant domain that comprises a C214S amino acid substitution according to the EU numbering convention.

In some aspects of any of the IgG2 modified Fc, the Fc comprises a C219S amino acid substitution according to the EU numbering convention. In some aspects of any of the IgG2 modified Fc, the antibody has an IgG2 isotype with a Kappa light chain constant domain that comprises a C214S amino acid substitution according to the EU numbering convention.

In some aspects of any of the IgG2 modified Fc, the Fc comprises an IgG2 isotype heavy chain constant domain 1 (CH1) and hinge region (White et al. Cancer Cell 27:138-148 (2015)). In certain aspects of any of the IgG2 modified Fe, the IgG2 isotype CH1 and hinge region comprise the amino acid sequence of 118-230 according to EU numbering. In some aspects of any of the IgG2 modified Fc, the antibody Fc region comprises a S267E amino acid substitution, a L328F amino acid substitution, or both, and/or a N297A or N297Q amino acid substitution according to the EU numbering convention.

In some aspects of any of the IgG2 modified Fc, the Fc further comprises one or more amino acid substitution at positions E430G, E430S, E430F, E430T, E345K, E345Q, E345R, E345Y, S440Y, and S440W according to EU numbering. In some aspects of any of the IgG2 modified Fc, the Fc may further comprise one or more mutations to enhance the antibody half-life in human serum (e.g., one or more (including all) of M252Y, S254T, and T256E mutations according to the EU numbering convention). In some aspects of any of the IgG2 modified Fc, the Fc may further comprise A330S and P331S.

In some aspects of any of the IgG2 modified Fc, the Fc is an IgG2/4 hybrid Fc. In some aspects, the IgG2/4 hybrid Fc comprises IgG2 aa 118 to 260 and IgG4 aa 261 to 447. In some aspects of any IgG2 modified Fc, the Fc comprises one or more amino acid substitutions at positions H268Q, V309L, A330S, and P331S according to EU numbering.

In some aspects of any of the IgG1 and/or IgG2 modified Fc, the Fc comprises one or more additional amino acid substitutions selected from A330L, L234F; L235E, or P331S according to EU numbering; and any combination thereof.

In certain aspects of any of the IgG1 and/or IgG2 modified Fc, the Fc comprises one or more amino acid substitutions at a residue position selected from C127S, L234A, L234F, L235A, L235E, S267E, K322A, L328F, A330S, P331S, E345R, E430G, S440Y, and any combination thereof according to EU numbering. In some aspects of any of the IgG1 and/or IgG2 modified Fc, the Fc comprises an amino acid substitution at positions E430G, L243A, L235A, and P331S according to EU numbering. In some aspects of any of the IgG1 and/or IgG2 modified Fc, the Fc comprises an amino acid substitution at positions E430G and P331S according to EU numbering. In some aspects of any of the IgG1 and/or IgG2 modified Fc, the Fc comprises an amino acid substitution at positions E430G and K322A according to EU numbering. In some aspects of any of the IgG1 and/or IgG2 modified Fc, the Fc comprises an amino acid substitution at positions E430G, A330S, and P331S according to EU numbering. In some aspects of any of the IgG1 and/or IgG2 modified Fe, the Fc comprises an amino acid substitution at positions E430G, K322A, A330S, and P331S according to EU numbering. In some aspects of any of the IgG1 and/or IgG2 modified Fc, the Fc comprises an amino acid substitution at positions E430G, K322A, and A330S according to EU numbering. In some aspects of any of the IgG1 and/or IgG2 modified Fc, the Fc comprises an amino acid substitution at positions E430G, K322A, and P331S according to EU numbering. In some aspects of any of the IgG1 and/or IgG2 modified Fc, the Fc comprises an amino acid substitution at positions S267E and L328F according to EU numbering. In some aspects of any of the IgG1 and/or IgG2 modified Fc, the Fc comprises an amino acid substitution at position C127S according to EU numbering. In some aspects of any of the IgG1 and/or IgG2 modified Fc, the Fc comprises an amino acid substitution at positions E345R, E430G and S440Y according to EU numbering.

In some aspects of any of the antibodies provided herein, the modified antibody Fc is an IgG4 modified Fc. In some aspects, the IgG4 modified Fc comprises one or more modifications. For example, in some aspects, the IgG4 modified Fc comprises one or more amino acid substitutions (e.g., relative to a wild-type Fc region of the same isotype). In some aspects of any of the IgG4 modified Fc, the one or more amino acid substitutions are selected from L235A, G237A, S229P, L236E (Reddy et al. J Immunol 164:1925-1933(2000)), S267E, E318A, L328F, M252Y, S254T, and/or T256E according to the EU numbering convention. In some aspects of any of the IgG4 modified Fc, the Fc may further comprise L235A, G237A, and E318A according to the EU numbering convention. In some aspects of any of the IgG4 modified Fc, the Fc may further comprise S228P and L235E according to the EU numbering convention. In some aspects of any of the IgG4 modified Fc, the IgG4 modified Fc may further comprise S267E and L328F according to the EU numbering convention.

In some aspects of any of the IgG4 modified Fc, the IgG4 modified Fc comprises may be combined with an S228P mutation according to the EU numbering convention (Angal et al. Mol Immunol. 30:105-108 (1993)) and/or with one or more mutations described in (Peters et al. J Biol Chem. 287(29):24525-33 (2012)) to enhance antibody stabilization.

In some aspects of any of the IgG4 modified Fc, the IgG4 modified Fc may further comprise one or more mutations to enhance the antibody half-life in human serum (e.g., one or more (including all) of M252Y, S254T, and T256E mutations according to the EU numbering convention).

In some aspects of any of the IgG4 modified Fc, the Fc comprises L235E according to EU numbering. In certain aspects of any of the IgG4 modified Fc, the Fc comprises one or more amino acid substitutions at a residue position selected from C127S, F234A, L235A, L235E, S267E, K322A, L328F, E345R, E430G, S440Y, and any combination thereof, according to EU numbering. In some aspects of any of the IgG4 modified Fc, the Fc comprises an amino acid substitution at positions E430G, L243A, L235A, and P331S according to EU numbering. In some aspects of any of the IgG4 modified Fc, the Fc comprises an amino acid substitution at positions E430G and P331S according to EU numbering. In some aspects of any of the IgG4 modified Fc, the Fc comprises an amino acid substitution at positions E430G and K322A according to EU numbering. In some aspects of any of the IgG4 modified Fc, the Fc comprises an amino acid substitution at position E430 according to EU numbering. In some aspects of any of the IgG4 modified Fc, the Fc region comprises an amino acid substitution at positions E430G and K322A according to EU numbering. In some aspects of any of the IgG4 modified Fc, the Fc comprises an amino acid substitution at positions S267E and L328F according to EU numbering. In some aspects of any of the IgG4 modified Fc, the Fc comprises an amino acid substitution at position C127S according to EU numbering. In some aspects of any of the IgG4 modified Fc, the Fc comprises an amino acid substitution at positions E345R, E430G and S440Y according to EU numbering.

### (7) Other Antibody Modifications

In some aspects of any of the antibodies, the antibody is a derivative. The term “derivative” refers to a molecule that includes a chemical modification other than an insertion, deletion, or substitution of amino acids (or nucleic acids). In certain aspects, derivatives comprise covalent modifications, including, but not limited to, chemical bonding with polymers, lipids, or other organic or inorganic moieties. In certain aspects, a chemically modified antigen binding protein can have a greater circulating half-life than an antigen binding protein that is not chemically modified. In certain aspects, a chemically modified antigen binding protein can have improved targeting capacity for desired cells, tissues, and/or organs. In some aspects, a derivative antigen binding protein is covalently modified to include one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain aspects, a derivative antigen binding protein comprises one or more polymer, including, but not limited to, monomethoxy-polyethylene glycol, dextran, cellulose, copolymers of ethylene glycol/propylene glycol, carboxymethylcellulose, polyvinyl pyrrolidone, poly-1, 3-dioxolane, poly-1,3,6-trioxane, ethylene/maleic anhydride copolymer, polyaminoacids (either homopolymers or random copolymers), poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain aspects, a derivative is covalently modified with polyethylene glycol (PEG) subunits. In certain aspects, one or more water-soluble polymer is bonded at one or more specific position, for example at the amino terminus, of a derivative. In certain aspects, one or more water-soluble polymer is randomly attached to one or more side chains of a derivative. In certain aspects, PEG is used to improve the therapeutic capacity for an antigen binding protein. In certain aspects, PEG is used to improve the therapeutic capacity for a humanized antibody. Certain such methods are discussed, for example, in U.S. Pat. No. 6,133,426, which is hereby incorporated by reference for any purpose.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. Adv. Drug Res., 15:29 (1986); and Evans et al. J. Med. Chem., 30:1229 (1987), which are incorporated herein by reference for any purpose. Such compounds are often developed with the aid of computerized molecular modeling. Peptide mimetics that are structurally similar to therapeutically useful peptides can be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain aspects to generate more stable peptides. In addition, constrained peptides comprising a consensus sequence or a substantially identical consensus sequence variation can be generated by methods known in the art (Rizo and Gierasch Ann. Rev. Biochem., 61:387 (1992), incorporated herein by reference for any purpose); for example, by adding internal cysteine residues capable of forming intramolecular disulfide bridges which cyclize the peptide.

Drug conjugation involves coupling of a biological active cytotoxic (anticancer) payload or drug to an antibody that specifically targets a certain tumor marker (e.g. a polypeptide that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue OncLive Jul. 5, 2012; ADC Review on antibody-drug conjugates; and Ducry et al. Bioconjugate Chemistry 21 (1):5-13 (2010).

### III. Nucleic Acids, Vectors, and Host Cells

Anti-MerTK antibodies of the present disclosure may be produced using recombinant methods and compositions, e.g., as described in U.S. Pat. No. 4,816,567. In some aspects, isolated nucleic acids having a nucleotide sequence encoding any of the anti-MerTK antibodies of the present disclosure are provided. Such nucleic acids may encode an amino acid sequence comprising the V_{L} and/or an amino acid sequence comprising the V_{H} of the anti-MerTK antibody (e.g., the light and/or heavy chains of the antibody). In some aspects, one or more vectors (e.g., expression vectors) comprising such nucleic acids are provided. In some aspects, a host cell comprising such nucleic acid is also provided. In some aspects, the host cell comprises (e.g., has been transduced with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In some aspects, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-MerTK antibody of the present disclosure are provided. In some aspects, the method comprises culturing a host cell of the present disclosure comprising a nucleic acid encoding the anti-MerTK antibody, under conditions suitable for expression of the antibody. In some aspects, the antibody is subsequently recovered from the host cell (or host cell culture medium).

For recombinant production of an anti-MerTK antibody of the present disclosure, a nucleic acid encoding the anti-MerTK antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors comprising a nucleic acid sequence encoding any of the anti-MerTK antibodies of the present disclosure, or cell-surface expressed fragments or polypeptides thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mpl8, mpl9, pBR322, pMB9, ColEl, pCR1, RP4, phage DNAs, and shuttle vectors such as pSA3 and pAT28. These and many other cloning vectors are available from commercial vendors such as BioRad, Strategene, and Invitrogen.

Suitable host cells for cloning or expression of antibody-encoding vectors include prokaryotic or eukaryotic cells. For example, anti-MerTK antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross Nat. Biotech. 22:1409-1414 (2004); and Li et al. Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al. Annals N.Y. Acad. Sci. 383:44-68 (1982); MRC 5 cells; and FS4 cells. Other useful mammalian host cell lines include Chinese hamster ovary (CHO) cells, including DHFR− CHO cells (Urlaub et al. Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### IV. Pharmaceutical Compositions/Formulations

Provided herein are pharmaceutical compositions and/or pharmaceutical formulations comprising the anti-MerTK antibodies of the present disclosure and a pharmaceutically acceptable carrier.

In some aspects, pharmaceutically acceptable carrier preferably are nontoxic to recipients at the dosages and concentrations employed. The pharmaceutical compositions and/or pharmaceutical formulations to be used for in vivo administration can be sterile. This is readily accomplished by filtration through e.g., sterile filtration membranes.

Pharmaceutical composition and/or pharmaceutical formulations provided herein are useful as a medicament, e.g., for treating an autoimmune disorder.

### V. Therapeutic and Prophylactic Uses

As disclosed herein, anti-MerTK antibodies of the present disclosure may be used for preventing, reducing risk, or treating diseases and disorders. In some aspects, the present disclosure provides methods for preventing, reducing risk, or treating an autoimmune disorder in an individual, such as, for example, multiple sclerosis, comprising administering to the individual a therapeutically effective amount of an anti-MerTK antibody of the present disclosure.

MerTK has been associated with multiple sclerosis; SNP polymorphisms in MerTK are associated with multiple sclerosis susceptibility. Accordingly, modulating the activity of MerTK with an anti-MerTK antibody of the present disclosure is an effective means of preventing or treating multiple sclerosis.

In certain aspects, provided herein are methods for treating multiple sclerosis in a subject in need thereof, the method comprising administering to the subject an anti-MerTK antibody of the present disclosure, or a pharmaceutical composition comprising an anti-MerTK antibody of the present disclosure. In some aspects, a method is provided for treating multiple sclerosis in a subject in need thereof, the method comprising administering to the subject an anti-MerTK antibody of the present disclosure, wherein the anti-MerTK antibody increases phagocytosis of myelin. In some aspects, a method is provided for treating multiple sclerosis in a subject in need thereof, the method comprising administering to the subject an anti-MerTK antibody of the present disclosure, wherein the anti-MerTK antibody increases phosphorylation of MerTK.

Several types of multiple sclerosis (MS) have been described. Relapsing-remitting MS (RRMS), primary progressive MS (PPMS), secondary progressive MS (SPMS), and clinically isolated syndrome (CIS). Relapsing-remitting MS is characterized by unpredictable relapses followed by periods of months to years of relative quite (remission) with no signs of disease activity. This describes the initial course of approximately 80% of individuals with MS. The relapsing-remitting subtype of MS usually begins with a clinically isolated syndrome, in which an individual has an attack suggestive of demyelination, but does not yet fulfill the criteria for MS. 30-70% of individuals who experience CIS later develop MS.

Primary progressive MS occurs in approximately 10-20% of individuals, with no remission after the initial symptoms. It is characterized by progression of disability from onset, with no, or only occasional and minor, remissions and improvements. Secondary progressive MS occurs in around 65% of those individuals with initial relapsing-remitting MS, who eventually have progressive neurologic decline between acute attacks without any definite periods of remission, although occasional relapses and minor remissions may appear.

In some aspects, an anti-MerTK antibody of the present disclosure is effective at decreasing the number of relapses in RRMS. In some aspects, an anti-MerTK antibody of the present disclosure is effective at decreasing the frequency of relapses in RRMS. In some aspects, an anti-MerTK antibody of the present disclosure is effective at decreasing the number and frequency of relapses in RRMS. In some aspects, an anti-MerTK antibody of the present disclosure is effective at preventing or reducing conversion from RRMS to SPMS. In some aspects, an anti-MerTK antibody of the present disclosure is effective at inhibiting or reducing disease progression in PPMS.

MerTK mutations are associated with various retinal ganglia degenerative disorders, including retinitis pigmentosa. Often, such retinal disorders are associated with reduction in the ability of retinal pigment epithelial (RPE) cells to phagocytose photoreceptor outer segments, which leads to accumulation of debris separating photoreceptors from RPE cells, resulting in their degradation and loss of vision. Additionally, mutations of the MerTK gene are associated with loss of night vision in early childhood, gradual constriction of the visual field, and eventual loss of visual acuity before adulthood (Lorach et al, 2018, Nature Scientific Reports, 8:11312). In some aspects, an anti-MerTK antibody of the present disclosure is effective at treating a retinal ganglia degenerative disorder. In some aspects, an anti-MerTK antibody of the present disclosure is effective at increasing phagocytosis of photoreceptor outer segments. In other aspects, an anti-MerTK antibody of the present disclosure is effective at treating retinitis pigmentosa.

In some aspects, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some aspects, the subject or individual is a human.

An antibody provided herein (and any additional therapeutic agent) can be administered by any suitable means.

### VI. Diagnostic Uses

In some aspects of any of the antibodies, any of the anti-MerTK antibodies provided herein is useful for detecting the presence of MerTK in a sample or an individual. The term “detecting” as used herein encompasses quantitative or qualitative detection. Provided herein are methods of using the antibodies of this disclosure for diagnostic purposes, such as the detection of MerTK in an individual or in tissue samples derived from an individual. In some aspects, the individual is a human. In some aspects, the tissue sample is phagocytic cells (e.g., macrophages, dendritic cells), tumor tissue, cancer cells, etc.

The detection method may involve quantification of the antigen-bound antibody. Antibody detection in biological samples may occur with any method known in the art, including immunofluorescence microscopy, immunocytochemistry, immunohistochemistry, ELISA, FACS analysis, immunoprecipitation, or micro-positron emission tomography. In certain aspects, the antibody is radiolabeled, for example with ¹⁸F and subsequently detected utilizing micro-positron emission tomography analysis. Antibody-binding may also be quantified in a patient by non-invasive techniques such as positron emission tomography (PET), X-ray computed tomography, single-photon emission computed tomography (SPECT), computed tomography (CT), and computed axial tomography (CAT).

### VII. Articles of Manufacture

Provided herein are articles of manufacture (e.g., kit) comprising an anti-MerTK antibody described herein. Article of manufacture may include one or more containers comprising an antibody described herein. Containers may be any suitable packaging including, but is not limited to, vials, bottles, jars, flexible packaging (e.g., sealed Mylar or plastic bags), and the like. The containers may be unit doses, bulk packages (e.g., multi-dose packages) or sub-unit doses.

In some aspects, the kits may further comprise a second agent. In some aspects, the second agent is a pharmaceutically-acceptable buffer or diluting agent.

In some aspects of any of the articles of manufacture, the article of manufactures further comprises instructions for use in accordance with the methods of this disclosure. The instructions generally include information as to dosage, dosing schedule, and route of administration for the intended treatment. In some aspects, these instructions comprise a description of administration of the isolated antibody of the present disclosure (e.g., an anti-MerTK antibody described herein) to prevent, reduce risk, or treat an individual having a disease, disorder, or injury, such as for example cancer, according to any methods of this disclosure.

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Production of his-Conjugated and Murine Fc-Conjugated MerTK Polypeptides

Human, cynomolgus (cyno), and murine MerTK polypeptides containing polyHis or TEVS/Thrombin/murine IgG2a-Fc tagged fusion proteins for use in the generation and characterization of anti-MerTK antibodies of the present disclosure were generated as follows. Nucleic acids encoding the extracellular domains (ECDs) of human MerTK (SEQ ID NO:2), cyno MerTK (SEQ ID NO:3), and murine MerTK (SEQ ID NO:4) were each cloned into a mammalian expression vector containing a nucleic acid encoding a heterologous signal peptide as well as containing either a PolyHis Fe tag or TEVS/Thrombin/murine IgG2a Fe tag.

The amino acid sequences of human MerTK, human MerTK extracellular domain, cyno MerTK extracellular domain, and murine MerTK extracellular domain are set forth below.

Human MerTK amino acid sequence (SEQ ID NO:1):

MGPAPLPLLLGLFLPALWRRAITEAREEAKPYPLFPGPFPGSLQTDHTP
LLSLPHASGYQPALMFSPTQPGRPHTGNVAIPQVTSVESKPLPPLAFKH
TVGHIILSEHKGVKFNCSISVPNIYQDTTISWWKDGKELLGAHHAITQF
YPDDEVTAIIASFSITSVQRSDNGSYICKMKINNEEIVSDPIYIEVQGL
PHFTKQPESMNVTRNTAFNLTCQAVGPPEPVNIFWVQNSSRVNEQPEKS
PSVLTVPGLTEMAVFSCEAHNDKGLTVSKGVQINIKAIPSPPTEVSIRN
STAHSILISWVPGFDGYSPFRNCSIQVKEADPLSNGSVMIFNTSALPHL
YQIKQLQALANYSIGVSCMNEIGWSAVSPWILASTTEGAPSVAPLNVTV
FLNESSDNVDIRWMKPPTKQQDGELVGYRISHVWQSAGISKELLEEVGQ
NGSRARISVQVHNATCTVRIAAVTRGGVGPFSDPVKIFIPAHGWVDYAP
SSTPAPGNADPVLIIFGCFCGFILIGLILYISLAIRKRVQETKFGNAFT
EEDSELVVNYIAKKSFCRRAIELTLHSLGVSEELQNKLEDVVIDRNLLI
LGKILGEGEFGSVMEGNLKQEDGTSLKVAVKTMKLDNSSQREIEEFLSE
AACMKDFSHPNVIRLLGVCIEMSSQGIPKPMVILPFMKYGDLHTYLLYS
RLETGPKHIPLQTLLKFMVDIALGMEYLSNRNFLHRDLAARNCMLRDDM
TVCVADFGLSKKIYSGDYYRQGRIAKMPVKWIAIESLADRVYTSKSDVW
AFGVTMWEIATRGMTPYPGVQNHEMYDYLLHGHRLKQPEDCLDELYEIM
YSCWRTDPLDRPTFSVLRLQLEKLLESLPDVRNQADVIYVNTQLLESSE
GLAQGSTLAPLDLNIDPDSIIASCTPRAAISVVTAEVHDSKPHEGRYIL
NGGSEEWEDLTSAPSAAVTAEKNSVLPGERLVRNGVSWSHSSMLPLGSS
LPDELLFADDSSEGSEVLM

Human MerTK ECD amino acid sequence (SEQ ID NO:2):

MGPAPLPLLLGLFLPALWRRAITEAREEAKPYPLFPGPFPGSLQTDHTP
LLSLPHASGYQPALMFSPTQPGRPHTGNVAIPQVTSVESKPLPPLAFKH
TVGHIILSEHKGVKFNCSISVPNIYQDTTISWWKDGKELLGAHHAITQF
YPDDEVTAIIASFSITSVQRSDNGSYICKMKINNEEIVSDPIYIEVQGL
PHFTKQPESMNVTRNTAFNLTCQAVGPPEPVNIFWVQNSSRVNEQPEKS
PSVLTVPGLTEMAVFSCEAHNDKGLTVSKGVQINIKAIPSPPTEVSIRN
STAHSILISWVPGFDGYSPFRNCSIQVKEADPLSNGSVMIFNTSALPHL
YQIKQLQALANYSIGVSCMNEIGWSAVSPWILASTTEGAPSVAPLNVTV
FLNESSDNVDIRWMKPPTKQQDGELVGYRISHVWQSAGISKELLEEVGQ
NGSRARISVQVHNATCTVRIAAVTRGGVGPFSDPVKIFIPAHGWVDYAP
SSTPAPGNADPVLII

Cyno MerTK ECD amino acid sequence (SEQ ID NO:3):

MGLAPLPLPLLLGLFLPALWSRAITEAREEAKPYPLFPGPLPGSLQTDH
TSLLSLPHTSGYQPALMFSPTQPGRPYTGNVAIPRVTSAGSKLLPPLAF
KHTVGHIILSEHKDVKFNCSISVPNIYQDTTISWWKDGKELLGAHHAIT
QFYPDDEVTAIIASFSITSVQRSDNGSYICKMKINNEEIVSDPIYIEVQ
GLPHFTKQPESMNVTRNTAFNLTCQAVGPPEPVNIFWVQNSSRVNEQPE
KSPSVLTVPGLTEMAVFSCEAHNDKGLTVSKGVQINIKAIPSPPTEVSI
HNSTAHSILISWVPGFDGYSPFRNCSVQVKEVDPLSNGSVMIFNTSASP
HMYQIKQLQALANYSIGVSCMNEIGWSAVSPWILASTTEGAPSVAPLNV
TVFLNESRDNVDIRWMKPLTKRQAGELVGYRISHVWQSAGISKELLEEV
GQNNSRAQISVQVHNATCTVRIAAVTKGGVGPFSDPVKIFIPAHGWVDH
APSSTPAPGNADPVLII

Murine MerTK ECD amino acid sequence (SEQ ID NO:4):

MVLAPLLLGLLLLPALWSGGTAEKWEETELDQLFSGPLPGRLPVNHRPF
SAPHSSRDQLPPPQTGRSHPAHTAAPQVTSTASKLLPPVAFNHTIGHIV
LSEHKNVKFNCSINIPNTYQETAGISWWKDGKELLGAHHSITQFYPDEE
GVSIIALFSIASVQRSDNGSYFCKMKVNNREIVSDPIYVEVQGLPYFIK
QPESVNVTRNTAFNLTCQAVGPPEPVNIFWVQNSSRVNEKPERSPSVLT
VPGLTETAVFSCEAHNDKGLTVSKGVHINIKVIPSPPTEVHILNSTAHS
ILVSWVPGFDGYSPLQNCSIQVKEADRLSNGSVMVENTSASPHLYEIQQ
LQALANYSIAVSCRNEIGWSAVSPWILASTTEGAPSVAPLNITVFLNES
NNILDIRWTKPPIKRQDGELVGYRISHVWESAGTYKELSEEVSQNGSWA
QIPVQIHNATCTVRIAAITKGGIGPFSEPVNIIIPEHSKVDYAPSSTPA
PGNTDSM

The human, cyno, and murine MerTK nucleic acid fusion constructs were transiently transfected into HEK293 cells. The recombinant fusion polypeptides were purified from the supernatants of the cells using Mabselect resin (GE Healthcare, Cat #17519902) following the manufacturer's instructions. Additionally, commercially available DDDDK-tagged human MerTK fusion polypeptide (Sino Biological, Wayne, PA, Cat #10298-HCCH) or human IgG1 Fc-tagged murine MerTK fusion proteins (R&D systems, Minneapolis, MA, Cat #591-MR-100) were also used for anti-MerTK antibody characterization as described below.

### Example 2: Generation of Human and Murine MerTK Overexpressing CHO Cell Lines

Human MerTK and murine MerTK overexpressing CHO cell lines were prepared as follows. Human MerTK open reading frame (ORF) clone Lentivirus particle (Cat #RC215289L4V) and mouse MerTK ORF clone Lentivirus particle (Cat #MR225392L4V) (Origene, Rockville, MD) (both mGFP-tagged) were used for preparing human MerTK overexpressing CHO-K1 and murine MerTK overexpressing CHO-K1 stable cell line generation, respectively.

CHO cells were cultured in F12-K media (ATCC, Cat #ATCC 30-2004) containing 10% FBS (Gibco) until >80% confluent. The cells were then dissociated with Trypsin buffer (0.25% EDTA/Trypsin, Gibco, Cat #25200056) and plated at 70-80% confluency in 6-well plates 24 hours prior to transduction with either the human or murine MerTK lentivirus construct. The following day, cells were incubated with the lentiviral particle at 4° C. for 2 hours, and then the plates were incubated at 37° C. in 5% CO₂. Two days later, puromycin (Invivogen, San Diego, CA, Cat #ant-pr-1) was added for selection; selected puromycin-resistant cells were frozen in Cell Recovery Freezing Medium (Gibco, Cat #12648010) for subsequent use.

For FACS analysis of these cell lines, human MerTK overexpressing CHO cells (CHO-huMerTK OE cells) and mouse MerTK overexpressing CHO cells (CHO-muMerTK OE cells) generated as descried above were plated at 1-2×10⁵ cells per well in 96-well U-bottom plates and incubated with a commercially available mouse anti-human MerTK monoclonal antibody “H1” (BioLegend, Clone: 590H11G1E3, Cat #367608, San Diego, CA) or a commercially available rat anti-mouse MerTK monoclonal antibody (ThermoFisher, Clone: DS5MMER, Cat #12-5751-82) for 30 minutes on ice. Cells were rinsed twice with ice-cold FACS buffer (2% FBS+PBS) and then incubated with APC-conjugated goat anti-mouse antibody (Jackson ImmunoResearch, West Grove, PA, Cat #115-606-071) or goat-anti-rat antibody (Jackson ImmunoResearch, Cat #112-606-071) for 30 minutes on ice. Following the secondary antibody incubation, the cells were washed with ice-cold FACS buffer and then resuspended in a final volume of 50-200 μl of FACS buffer containing 0.25 μl/well propidium iodide (BD, Cat #556463). Analysis was performed using a FACS CantoII system (BD Biosciences).

The resulting human MerTK and murine MerTK overexpressing CHO cell lines were used for subsequent studies to characterize anti-MerTK antibodies as described below.

### Example 3: Generation of Anti-MerTK Hybridoma Antibodies

In order to obtain antibodies against MerTK, the following experiments were performed to generate anti-MerTK hybridomas. BALB/c mice (Charles River Laboratories, Wilmington, MA) or MerTK knock-out (KO) mice (Jackson Laboratories, Bar Harbor, ME) were immunized twice a week by subcutaneous or intraperitoneal injections of purified extracellular domain polypeptides of human, cyno, and mouse MerTK (obtained as described above in Example 1) with or without adjuvant. A total of 8 injections were performed over 4 weeks. Three days following the final injection, spleens and lymph nodes were harvested from the mice for hybridoma cell line generation.

Lymphocytes from the spleens and lymph nodes of the immunized mice were isolated and then fused with P3X63Ag8.653 (CRL-1580, American Type Culture Collection, Rockville, MD) or SP2/mIL-6 (CRL-2016, American Type Culture Collection, Rockville, MD) mouse myeloma cells via electrofusion (Hybrimmune, BTX, Holliston, MA) and incubated at 37° C., 5% CG2, overnight in Clonacell-HY Medium C (STEMCELL Technologies, Vancouver, BC, Canada, Cat #03803). The following day, the fused cells were centrifuged and resuspended in 10 ml of ClonaCell-HY Medium C with anti-mouse IgG Fc-FITC (Jackson ImmunoResearch, West Grove, PA) and then gently mixed with 90 ml of methylcellulose-based ClonaCell-HY Medium D (STEMCELL Technologies, Cat #03804) containing HAT components. The cells were plated into Nunc OmniTrays (Thermo Fisher Scientific, Rochester, NY) and allowed to grow at 37° C., 5% CO2 for seven days. Fluorescent colonies were then selected and transferred into 96-well plates containing Clonacell-HY Medium E (STEMCELL Technologies, Cat #03805) using a Clonepix 2 (Molecular Devices, Sunnyvale, CA). In total, 1,728 IgG-secreting hybridoma clones were selected. After six days of culture, tissue culture supernatants from the hybridomas were screened by FACS analysis for specificity to bind human or mouse MerTK as described below.

### Example 4: Screening of Anti-MerTK Antibody Hybridoma Supernatants by FACS

Hybridoma culture supernatants from 1,728 hybridomas obtained as described above were screened for their ability to bind MerTK on various cell types, including CHO cells stably overexpressing human MerTK (CHO-huMerTK OE cells) or stably overexpressing mouse MerTK (CHO-muMerTK OE cells) (generated as described above), and CHO parental cells; U937 cells (ATCC CRL-1593.2), SK-MEL-5 cells (ATCC HTB-70) (which endogenously express human MerTK), J774A.1 cells (ATCC TIB-67) (which endogenously express mouse MerTK), and A375 cells (ATCC CRL-1619).

For screening of the hybridoma cell culture supernatants, a multiplexed FACS experimental design was utilized to determine binding of anti-MerTK antibodies to these multiple cell lines. Briefly, cells were stained with various concentrations and combinations of CellTrace cell proliferation dyes CFSE and Violet (ThermoFisher, Cat #C34554 and Cat #34557, respectively) to create uniquely barcoded cell populations. 70,000 cells of each barcoded cell type were aliquoted into 96-well U-bottom plates and incubated with 501 of hybridoma cell culture supernatant or 5 μg/ml of commercially available purified mouse anti-human MerTK monoclonal antibody (BioLegend, Cat #367602; serving as a positive anti-MerTK antibody) on ice for 30 minutes. After this primary anti-MerTK hybridoma supernatant incubation with the various MerTK expressing cell types, the supernatants were removed via centrifugation, the cells were washed twice with 175 μl of ice-cold FACS buffer (PBS+1% FBS+2 mM EDTA), and the cells were then further incubated on ice for 20 minutes with anti-mouse IgG Fc-allophycocyanin (APC) (Jackson Labs, Cat #115-136-071) (diluted 1:1000). Following this secondary antibody incubation, the cells were again washed twice with ice-cold FACS buffer and resuspended in a final volume of 30 μl of FACS buffer containing 0.25 μl/well propidium iodide (BD Biosciences, Cat #556463). Binding intensity on cells was analyzed using the FACS Canto system (BD Biosciences), with sorting gates drawn to exclude dead (i.e., propidium iodide-positive) cells. The ratio of APC Mean Fluorescence Intensity (MFI) on each barcoded cell population was determined for each anti-MerTK hybridoma supernatant tested.

From this specific hybridoma supernatant screen, a total of 308 anti-MerTK hybridoma clones were identified that displayed greater than 2-fold difference in binding (as determined by MFI) to cells stably overexpressing or endogenously expressing human or mouse MerTK compared to the binding observed on parental or negative control cell types. Anti-MerTK antibodies identified using this screen were further characterized as described below.

### Example 5: Screening of Anti-MerTK Antibody Hybridoma Supernatants by Recombinant MerTK Protein Binding Assay

Hybridoma culture supernatants from 1,728 hybridomas obtained as described above were screened for their ability to bind polyHis-tagged human, cyno, and mouse MerTK (prepared as described above in Example 1) as compared to binding to an irrelevant His-tagged control protein. Briefly, 96-well polystyrene plates were coated with 1 μg/ml of human, cyno, or mouse poly-His-tagged MerTK polypeptide in coating buffer (0.05M carbonate buffer, pH 9.6, Sigma, Cat #C3041) overnight at 4° C. Coated plates were then blocked with ELISA diluent (PBS+0.5% BSA+0.05% Tween20) for one hour and washed three times with 300 μl of PBST (PBS+0.05% Tween20, Thermo 28352). The hybridoma cell culture supernatants or two commercially available purified mouse anti-human MerTK monoclonal antibodies (BioLegend Cat #367602; R&D Cat #MAB8912) were added (50 μl/well) to each well. After 30 mins incubation (room temperature, with shaking), the plates were washed three times with 300 μl of PBST. Anti-mouse IgG Fc-HRP (Jackson Immunoresearch, Cat #115-035-071) secondary antibody was diluted 1:5000 in ELISA diluent, added to each well at 50 μl/well, and incubated for 30 minutes at room temperature with shaking. After a final set of washes (3×300 μl in PBST), 50 μl/well of TMB substrate (BioFx, Cat #TMBW-1000-01) was added to the wells. The reaction was then quenched after 5-10 mins with 50 μl/well of stop solution (BioFx, Cat #BSTP-1000-01). The quenched reaction wells were detected for absorbance at 650 nm with a BioTek Synergy Microplate Reader using GEN5 2.04 software. From this hybridoma supernatant screen, a total of 326 anti-MerTK hybridoma clones were identified that displayed greater than 10-fold difference in binding to recombinant MerTK over background. Anti-MerTK antibodies identified using this screen were further characterized as described below.

### Example 6: MerTK Ligand Gas6 and Ligand ProS Blocking Assay Using Anti-MerTK Hybridoma Supernatants

Anti-MerTK antibody hybridoma supernatants identified as described above were screened by ELISA to identify anti-MerTK antibodies that did not block binding of human Gas6 ligand to human MerTK and/or did not block binding of human ProS ligand to human MerTK. Briefly, rabbit anti-human IgG antibody (Jackson ImmunoResearch, Cat #309-005-008) was coated at 2 μg/ml onto high-protein binding plates at 4° C. overnight. After washing with 0.05% Tween20 in PBS three times, 5% BSA in PBS was added for 1 hour. Recombinant human MerTK-human Fe Chimera protein (R&D systems, Cat #391-MR-100) was added at 2 μg/ml for 1 hour and plates were washed before the addition of 40 μl of anti-MerTK hybridoma supernatants and 40 μl of His tag-conjugated recombinant Gas6 (R&D systems, Cat #885-GSB-050) at 3 μg/ml or His tag-conjugated recombinant ProS (R&D systems, Cat #9489-PS-100) at 20 μg/ml. After a further incubation for 1 hour, plates were washed and incubated for 1 hour with HRP-conjugated anti-6×His tagged antibody (Abcam, Cambridge, MA, Cat #ab1187). Plates were then washed and an HRP substrate, TMB, was added to develop the plates. The reaction was stopped by adding 50 μl 2N H₂SO₄ and the OD was measured using a spectrophotometer (BioTek).

A total of 308 anti-MerTK hybridoma supernatant clones were screened to identify anti-MerTK antibodies that did not block binding of human Gas6 ligand to human MerTK and/or did not block binding of human ProS ligand to human MerTK. Twenty-nine (29) anti-MerTK hybridoma clones blocked the binding of both ProS ligand and Gas6 ligand to recombinant human MerTK protein. One hundred forty-five (145) anti-MerTK hybridoma clones blocked binding of ProS ligand to human MerTK protein only and did not block the binding of Gas6 ligand to recombinant human MerTK protein. The remaining 134 of the 308 anti-MerTK hybridoma clones did not block either ProS ligand or Gas6 ligand binding to recombinant human MerTK protein in this assay. The hybridoma supernatants were characterized further as described below.

### Example 7: Efferocytosis Blocking Assay Using Anti-MerTK Hybridoma Supernatants

Anti-MerTK antibody hybridoma supernatants identified above as positive for MerTK binding reactivity were screened to identify anti-MerTK antibodies that did not block efferocytosis by human macrophages as follows. Human macrophages were differentiated from human monocytes for 7 days in the presence of human M-CSF. Macrophages were harvested (by scraping), resuspended in PBS, and plated on 96-well plates at 5×10⁴ cells/well. Cells were starved for 1 hour followed by the addition of anti-MerTK hybridoma supernatants to each well for 30 min at 37° C. Jurkat cells were treated with 1 μM staurosporin (SigmaAldrich) for 3 hours at 37° C. (to induce apoptosis) and labeled with pHrodo (ThermoFisher) for 30 min at room temperature. After washing with PBS, pHrodo labeled Jurkat cells were added into each well at 1:4 ratio (1 macrophage cell to 4 Jurkat cells) for 1 hour. The plates were washed with PBS, and then cells were stained with APC-conjugated anti-human CD14 for 30 minutes on ice in the dark. Cells were then washed twice in FACS buffer (PBS+2% FBS), and flow cytometry was performed on a BD FACS CantoII. Data were analyzed using FlowJo software.

In these experiments, efferocytosis-positive macrophages were identified by setting pHrodo CD14 double positive cells as an analysis gate and then applying this exact gate to all the samples. Baseline efferocytosis levels were established using macrophages cultured with media alone and this was set to 100% efferocytosis activity. Relative efferocytosis levels were calculated as a percent of efferocytosis observed in cells treated with media alone compared to that observed in cells treated with anti-MerTK hybridoma supernatants. In these experiments, the following additional anti-MerTK antibodies were used: mouse anti-human MerTK antibody H1 (BioLegend, Clone ID: 590H11G1E3, mouse IgG1) and human anti-human MerTK antibody M6 (disclosed in WO2016/106221).

Table 1 and Table 2 below show results from these efferocytosis experiments, shown as percent efferocytosis (media alone was set to 100% efferocytosis). In Table 1 below, exemplary hybridoma supernatants tested are indicated on the left and labeled as hybridoma supernatant ID number; these supernatants were from hybridoma clones identified from immunization of wildtype BALB/c mice. In Table 2 below, exemplary hybridoma supernatants tested are indicated on the left and labeled as hybridoma supernatant ID number; these supernatants were from hybridoma clones identified from immunization of MerTK KO mice. In both Table 1 and Table 2, antibody ID refers to anti-MerTK antibodies of the present disclosure that were selected for additional characterization and thus given a specific anti-MerTK antibody name, as indicated. Note that in comparison to efferocytosis in macrophages in the absence of antibody (media treatment alone), no significant change in efferocytosis was observed in cells treated with isotype control mouse IgG1 antibody.

Taken together, these results showed that certain anti-MerTK antibodies of the present disclosure are effective at increasing efferocytosis by macrophages. As efferocytosis is one aspect of phagocytosis activity, the results indicated that anti-MerTK antibodies of the present disclosure are effective at increasing phagocytosis by a phagocytic cell. These results also showed that anti-MerTK antibodies of the present disclosure did not reduce efferocytosis activity by macrophages by more than 4000 compared to that observed by macrophages in the absence of anti-MerTK antibody treatment (e.g., media alone control or isotype control antibody addition).

TABLE 1

| Hybridoma | Antibody | Donor | Donor | Donor | Donor |
|---|---|---|---|---|---|
| supernatant ID | ID | 689 | 692 | 805 | 806 |
| 1 | MTK-205 | 77.6 | 63.9 | 67.7 | 89.8 |
| 2 | MTK-231 | 70.6 | 74.4 | 81.9 | 84.0 |
| 3 | MTK-232 | 26.9 | 67.2 | 65.7 | 91.7 |
| | Media | 100.0 | 100.0 | 100.0 | 100.0 |
| | mIgG1 | 104.5 | 95.6 | 79.5 | 96.0 |
| | H1 | 31.2 | 16.5 | 59.8 | 57.9 |
| | M6 | 14.9 | n.a. | 60.2 | 35.1 |

n.a. data not available

TABLE 2

| Hybridoma | | | | | |
|---|---|---|---|---|---|
| supernatant | Antibody | Donor | Donor | Donor | Donor |
| ID | ID | 914 | 915 | 916 | 783 |
| 1 | MTK-201 | 71.4 | 51.0 | 50.0 | 78.6 |
| 2 | MTK-202 | 86.6 | 66.8 | 72.7 | 106.7 |
| 3 | MTK-203 | 88.7 | 59.1 | 72.7 | 113.0 |
| 4 | MTK-204 | 99.4 | 69.6 | 99.2 | 133.8 |
| 5 | MTK-206 | 87.5 | 71.7 | 93.0 | 110.4 |
| 6 | MTK-207 | 97.9 | 73.7 | 81.0 | 122.1 |
| 7 | MTK-208 | 77.7 | 55.1 | 64.5 | 55.5 |
| 8 | MTK-209 | 77.1 | 71.7 | 82.2 | 124.1 |
| 9 | MTK-210 | 80.1 | 67.6 | 66.9 | 100.0 |
| 10 | MTK-211 | 84.8 | 83.4 | 88.8 | 112.4 |
| 11 | MTK-212 | 101.8 | 81.0 | 100.8 | 136.5 |
| 12 | MTK-213 | 90.5 | 62.3 | 72.3 | 115.4 |
| 13 | MTK-214 | 74.4 | 53.4 | 67.8 | 78.6 |
| 14 | MTK-215 | 87.8 | 73.3 | 72.7 | 53.5 |
| 15 | MTK-216 | 69.3 | 66.0 | 69.8 | 121.1 |
| 16 | MTK-217 | 71.7 | 55.5 | 52.5 | 73.9 |
| 17 | MTK-218 | 86.9 | 68.4 | 81.0 | 108.7 |
| 18 | MTK-219 | 91.1 | 66.0 | 81.0 | 128.1 |
| 19 | MTK-220 | 112.8 | 87.4 | 118.2 | 139.1 |
| 20 | MTK-221 | 71.4 | 53.0 | 55.4 | 58.2 |
| 21 | MTK-222 | 88.7 | 74.1 | 79.3 | 112.7 |
| 22 | MTK-223 | 66.7 | 64.0 | 59.1 | 91.6 |
| 23 | MTK-224 | 68.8 | 59.9 | 86.8 | 106.0 |
| 24 | MTK-225 | 61.3 | 47.0 | 50.8 | 116.7 |
| 25 | MTK-226 | 77.1 | 73.7 | 74.8 | 116.7 |
| 26 | MTK-227 | 86.6 | 59.5 | 48.3 | 85.3 |
| 27 | MTK-228 | 64.6 | 64.8 | 64.0 | 90.0 |
| 28 | MTK-229 | 63.1 | 56.3 | 54.1 | 40.1 |
| 29 | MTK-230 | 58.0 | 50.6 | 57.4 | 114.4 |
| | Media alone | 100.0 | 100.0 | 100.0 | 100.0 |
| | M6 | 17.5 | 20.4 | 28.3 | 14.8 |
| | Isotype | 89.0 | 104.0 | 104.5 | 93.6 |
| | control | | | | |
| | H1 | 24.8 | 34.3 | 40.8 | 34.8 |

In Table 1 and Table 2, the percent of efferocytosis by human macrophages obtained from different donors is shown, with media alone (no antibody addition) set to 100% efferocytosis in both Tables. Although the degree of efferocytosis blocking (or non-blocking) activity by various anti-MerTK antibody hybridoma supernatants was different in macrophages obtained from each donor, the efferocytosis blocking (or non-blocking) trend for anti-MerTK antibodies was consistent among macrophages from different donors. As shown in Tables 1 and 2 above, anti-MerTK hybridoma supernatants of the present disclosure displayed lower ability to block or reduce efferocytosis by human macrophages compared to that observed by anti-MerTK antibody H1 and anti-MerTK antibody M6, both of which inhibit efferocytosis. These results indicated that certain anti-MerTK antibodies obtained as described herein were not effective at inhibiting or blocking efferocytosis by phagocytic cells.

### Example 8: Molecular Cloning of Anti-MerTK Antibodies

Anti-MerTK antibodies from the hybridomas described above were subcloned as follows. 5×10⁵ hybridoma cells were harvested and washed with PBS and then the cell pellets were flash frozen in dry ice and stored at −20° C. Total RNA was extracted by using RNeasy Mini Kit (QIAGEN, Cat #74104) following the manufacturer's protocol. cDNA was generated using Clontech's SMARTer RACE 5′/3′ Kit (Takara Bio USA, Cat #634859) following the manufacturer's protocol. Variable heavy and light immunoglobulin regions were cloned separately by touchdown PCR using the 5′ UPM primer provided in the RACE kit and reverse primers recognizing the heavy chain and light chain constant regions. The resulting PCR products were purified and ligated into a pCR2.1-TOPO cloning vector (TOPO TA cloning Kit, Invitrogen Cat #450641) and transformed into Escherichia coli (E. coli) cells. Transformed colonies were isolated and the variable heavy chain (VH) and variable light chain (VL) nucleic acids were sequenced for each corresponding hybridoma cell line. Following the sequence determinations, variable heavy chain regions and variable light chain regions were amplified by PCR using primers containing endonuclease restriction sites and then subcloned into pLEV-123 (LakePharma, San Carlos, CA) mammalian expression vector encoding human IgG1-Fc and IgG Kappa.

Amino acid sequences of the variable heavy chains and variable light chains of anti-MerTK antibodies of the present disclosure are provided below in Table 3. In Table 3, the CDR sequences (according to Kabat) are underlined.

TABLE 3

| | | SEQ | | SEQ |
|---|---|---|---|---|
| | | ID | | ID |
| Antibody | Heavy Chain Variable | NO: | Light Chain Variable | NO: |
| MTK-201 | QVQLRQSGPGLVQPSQSLSI | 5 | QIVLTQSPPIMSAFLGERVTM | 34 |
| | TCTVSGFSLSNYGLHWVRQS | | TCTASSSVSSSYLHWYRQMPG | |
| | PGKGLEWLGVIWSGGITDYN | | SSPKLWIYDTSNLASGVPARF | |
| | AAFISRLSIIKDNSRSQVFF | | SGSGSGTSYSLTISSMEAEDA | |
| | KMNSLQADDTAIYYCARKGH | | ATYYCHQYHHSPYTFGGGTKL | |
| | DPYAMDFWGQGTSVTVSS | | EIK | |
| MTK-202 | EVQLVESGGGLVKPGGSLKL | 6 | NIMMTQSPSSLAVSAGEKVTM | 35 |
| | SCAASGFTFSDFGMHWVRQV | | NCKSSQSVLYSSNQKNYLAWY | |
| | PEKGLEWVAFISGGSNTIYY | | QQKPGQSPKLLIYWASTRESG | |
| | TDTVKGRFTISRDNAKNTLF | | VPDRFTGSGSGTDFTLTISNI | |
| | LQMTSLRSEDTAMYYCVRNG | | QAEDLAIYYCHQYFFSWTFGG | |
| | NSFAYWGQGTLVTVSA | | GTKLEIK | |
| MTK-203 | QVQLQQPGAELVKPGASVKV | 7 | ETTVTQSPASLSVATGEKVTI | 36 |
| | SCKASGYTFTNYWMHWVKQR | | RCITSTDIGDDMNWYQQKPGE | |
| | PGQGLEWIGRIHPFDTETND | | PPKVLISEGNSLRPGVPSRFS | |
| | NQKFKGKATLTVDKSSSTAY | | SSGYGTDFVFTIENTLSEDVA | |
| | MQLSSLTSEDSAVYYCVIPA | | DYFCLQSDNLPLTFGSGTKLE | |
| | NGGFAYWGQGTLVTVSA | | IK | |
| MTK-204 | EVQLVESGGGLVKPGGSLKL | 8 | NIMMTQSPSSLAVSAGEKVTM | 37 |
| | SCAASGFTFSDYGMHWVRQA | | SCKSSQSVLYSSNQKNYLAWY | |
| | PEKGLEWVAYISSGTSTIYY | | QQKPGQSPKLLIQWASTRESG | |
| | EDTVKGRFTISRDNAKNTLF | | VPDRFTGSGSETDFTLTIRSV | |
| | LQMTSLRSEDTAMYYCARGG | | QAEDLAVYYCHQYFSSWTFGG | |
| | LLFDYWGQGTTLTVSS | | GTKLEIK | |
| MTK-205 | EVQLQQSGPELVKPGASVKI | 222 | DIVMTQSHKFMSTSVGDRVSI | 223 |
| | SCKTSGYTFTEYIMHWVKQS | | TCKASQDVGTSVAWYQQKPGH | |
| | HGKSLEWIGGINPNNLGIIY | | SPKLLIYWTSTRRTGVPDRFT | |
| | NQKFKGKATLTVDKSSSTAY | | GSGSGTDFTLTISNVQSEDLA | |
| | MELRSLTSEDSEVYYCARDG | | DYFCHQYNSYPLTFGAGTKLE | |
| | YFGFDYWGQGTTLKVSS | | LK | |
| MTK-206 | QVQLQQSGAELARPGASVKM | 9 | DIQMTQSPSSLSASLGERVSL | 38 |
| | SCKASGYTFTSYTMHWVKQR | | TCRASQDIGSNLNWLQQEPDG | |
| | PGQGLEWIGYINPSSGYIKN | | TIKRLIYATSSLESGVPKRFS | |
| | SQKFKDKATLTADKSSSTAY | | GSRSGSDYSLTISSLESEDFV | |
| | MQLSSLTSEDSAVYYCARTV | | DYYCLQYDSSPWTFGGGTKLE | |
| | YGYYGSSPDYWGQGTTLTVS | | IK | |
| | S | | | |
| MTK-207 | QVQLKESGPGLVAPSQSLSI | 10 | DIQMTQSPASLSASVGETVTI | 39 |
| | TCTVSGISLTSYGVSWVRQP | | TCRASGNIHNYLAWYQQKQGK | |
| | PGKGLEWLGIIWGDGSTNYH | | SPQLLVYNAKTLAAGVPSRFS | |
| | SALISRLSISKDNSKSQVFL | | GSGSGTQYSLKINSLQPEDFG | |
| | KLNSLQTDDTATYYCAKGRY | | SYYCQHFWSIPLTFGAGTKLE | |
| | GNYEDWYFDVWGTGTTVTVS | | LK | |
| | S | | | |
| MTK-208 | QVQLQQPGAELVVPGASVRL | 11 | QVVLTQSPAIMSASPGEKVTI | 40 |
| | SCKASGYTFINYWMHWVKQR | | TCSASSSVSYMLWFQQKPGTS | |
| | PGQGLEWIGEIDPSDSYANY | | PKLWIYSTSNLASGVPARFSG | |
| | NQKFKDKSTLSLDKSSSTAY | | SGSGTSYSLTISRLEAEDAAT | |
| | MQLSSLTSEDSAVYYCARCD | | YYCQQRSSYPPITFGAGTKLE | |
| | YYGRLDYWGQGTTLTVSS | | LK | |
| MTK-209 | QVQLQQPGAELVKPGASVKV | 12 | ETTVTQSPASLSMAMGEKATI | 41 |
| | SCKASGYTFTNYWIHWVKQR | | RCITSTDIDDKMNWYQQKPGK | |
| | PGQGLEWIGRIHPFDSEPNY | | PPKLLISAGNTLRPGVPSRFS | |
| | NQKFKGKATLTVDKSSSTAY | | SSGYGTDFVLTVENMLSEDVA | |
| | MHLTSLTSEDSAVYYCALAS | | DYYCLQGGALPLTFGSGTKLE | |
| | FAYWGQGTLVTVSA | | IK | |
| MTK-210 | QVQLQQSGPELVKPGASVKI | 13 | QIVLTQSPAIMSASPGVKVTM | 42 |
| | SCKASDYEFSRSWMNWVKQR | | TCSASSSVSYMHWYQQKPGSS | |
| | PGKGLEWIGRIYPGDGDTNY | | PRLLIYDTSNLASGVPVRFSG | |
| | NGKFKGKATLTADKSSNTAY | | SGSGTSYSLTISRMEAEDAAT | |
| | MQLNSLTSEDSAVYFCARKG | | YYCQQWSSYPPYTFGGGTKLE | |
| | GYYYSISNYFDYWGQGTTLT | | IK | |
| | VSS | | | |
| MTK-211 | QVQLQQSGAELMKPGASVKL | 14 | QIVLTQSPAVMSASPGEKVTM | 43 |
| | SCKATGYTFTGYWIEWVKQR | | TCSASSSITYMHWYQQKPGTS | |
| | PGHGLEWIGEILPGSGGSNY | | PKRWIYDTSKLASGVPARFSG | |
| | NEKFKGKATFTADTSSNTAY | | SGSGTSYFLTISSMEAEDVAT | |
| | IQLSSLTTEDSAIYCCARGE | | YYCHQRSTYPYTFGGGTKLEI | |
| | PSYFSDAGFAYWGQGTLVTV | | M | |
| | SA | | | |
| MTK-212 | QVQLQQSGPELVKPGASVKI | 15 | DIVMTQSHKFKSTSVGDRVSI | 44 |
| | SCKASGYAFSSSWVNWVKQR | | ACKASQDVGTSVAWYQQKPGK | |
| | PGKGLEWIGRIYPGDGDSNY | | SPKLLIYWASSRHTGVPDRFT | |
| | NGNFKGKATLTADKSSNTAY | | GSGSGTDFTLTISNVQSEDLT | |
| | LQLSSLTSEDSAVYFCARKS | | DYFCQQYSTYPYTFGGGTKLE | |
| | HFIYYTLDYWGQGTPVTVSS | | IK | |
| MTK-213 | QVQLQQPGAELVKPGASVKM | 16 | DIVMTQSHKFMSTSVGDRVSI | 45 |
| | SCKASGYTFTSYWITWVKQR | | TCKASQDVGDAVAWYQQKPGQ | |
| | PGQGHEWIGEIYPGIGSTNY | | TPKLLIYWASTRHTGVPDRFT | |
| | NEKFKSKATLTVDTSSRTAY | | GSGSGTDFTLTISNVQSEDLA | |
| | MQLSSLTSEDSAVYFCARGA | | DYFCQQYSSYLMYTFGGGTKL | |
| | YTKYYFDYWGQGTTLTVSS | | EIK | |
| MTK-214 | QVQLQQPGAELVKPGASVKV | 17 | DILLTQSPATLSVTPGETVSL | 46 |
| | SCKASGYTFTNYWMHWMKQR | | SCRASQSIYKKLHWFQQRSHR | |
| | PGQGLEWIGRIHPFDSDTNY | | SPRLLIKFASDSISGIPSRFT | |
| | NEKFTDKATLTVDKSSSTAY | | GSGSGTDYTLSINSVKPEDEA | |
| | MQLSSLTSEDSAVYYCAIGT | | IYYCLQGYSTPLTFGAGTKLE | |
| | LIYWGQGTTLTASS | | LK | |
| MTK-215 | EVQLVESGGGLVKPGGSLKL | 18 | DIVLTQSPATLSVTPGDSVSL | 47 |
| | SCAASGFIFSDYGMHWVRQA | | SCRASQSISNNLHWYQQKSHE | |
| | PEKGLEWVAYISSGSSTIYY | | SPRLLIKNPSQSISGIPSRFG | |
| | GDTVKGRFTISRDNAKNTLF | | ARGSGTDFTLIINSVETEDLG | |
| | LQMTSLRSEDTAMYYCAKGG | | VYFCQQSNSWPFTFGSGTKLE | |
| | RYGAMDYWGHGTSVTVSS | | IK | |
| MTK-216 | EVQLVESGGGLVKPGGSLKL | 19 | SIMMTQSPSSLAVSAGEKVTM | 48 |
| | SCAASGFTFSDYGMHWVRQA | | GCKSSQSVLYSANQQNYLAWY | |
| | PEKGLEWVAFISSGSTTIYY | | QQKPGQSLKLLIYWASTRESG | |
| | ADTVKGRFTISRDNAKNTLI | | VPDRFTGSGSGTDFTLTISSV | |
| | LQMTSLRSEDTAIYYCTRGG | | QAEDLAVYYCHQYFSSWTFGG | |
| | FHLDYWGQGTTLTVSS | | GTKLEIK | |
| MTK-217 | QVQLQQPGTEVVKPGASVKM | 20 | DILLTQSPAILSVSPGERVSF | 49 |
| | SCRASGYTFTSYWITWVKQR | | SCRASQTIGTSIHWYQQRTND | |
| | PGQGLEWIGDIHPGSGINNY | | SPRLLIKYASESISGIPSRFS | |
| | NEKFRSKATLTVDTSSSTVY | | GSGSGTDFTLSINSVESEDIA | |
| | MELSSLTSEDSAVYYCAKGG | | DYYCQQSNDWPYTFGGGTKLE | |
| | YYGSSYYVMDYWGQGTSVTV | | IK | |
| | SS | | | |
| MTK-218 | QVQLQQSGPELVKPGTSVKI | 21 | DAVMTQTPLSLPVSLGDQASI | 50 |
| | SCKASGYSFTSYYIHWVKQR | | SCRSSQSLENSNGHTYLHWYL | |
| | PGQGLDWIGWIFPGNRNTQY | | QKPGQSPQLLIYRVSNRFSGV | |
| | NEKFKGKATLTADTSSSTAY | | QDRFSGSGSGTDFTLKISRVE | |
| | MQLSSLTSEDSAVYYCARLG | | AEDLGVYFCLQVTHVPFTFGS | |
| | LIYALDYWGQGTSVTVSS | | GTKLEIK | |
| MTK-219 | QVQLQQSGADLMKPGASVKL | 22 | DIVLTQSPASLAVSLGQRATI | 51 |
| | SCKATGNTFTGYWIEWVKQR | | SCRASESVDSYGNSFMHWYQQ | |
| | PGHGLEWIGEILTGSGSTNY | | KPGQPPKLLIYRAYTLESGIP | |
| | NERFKGKATFTADTSSNTAY | | ARFSGSGSRTDFTLTINPVEA | |
| | MQLSSLTTEDSAIYYCAGSY | | DDVATYYCQQSNEDPRTFGGG | |
| | FTNSWFPYWGQGSLVTVSA | | TKLEIK | |
| MTK-220 | EVQLVESGGGLVKPGGSLKL | 23 | NIMLTQSPSSLAVSAGEKVTM | 52 |
| | SCAASGFTFSDYGMHWVRQA | | TCKSSQSVLYSSNQKNYLAWY | |
| | PEKGLEWVAFISGGSGTIYY | | QQKPGQSPKLLIYWASTRESG | |
| | EDTVKGRFTISRDNAKNTLF | | VPDRFTGSGSGTDFTLTISNV | |
| | LQMTSLRSEDTAMYYCTRGG | | QAEDLAVYYCHQYFSSWTFGG | |
| | NYFDYWGQGTTLTVSS | | GTKLEIK | |
| MTK-221 | QVTLKESGPGMLQSSQTLSL | 24 | DIQMTQSPSSLSASLGGKVTI | 53 |
| | TCSFSGFSLSTSGMGVSWIR | | TCKASQDIHKHIAWYQHKPGK | |
| | QPSGKGLEWLAHIYWDDDKR | | GPRLLIHYTSTLQPGIPSRFS | |
| | YNTSLQSRLTISKDTSRNQV | | GSGSGRDYSFSISNLEPEDLA | |
| | FLKITSVDTADTATYFCGRL | | TYYCLQYDNLWTFGGGTKLEI | |
| | GAYSAFPYYFDYWGQGTTLT | | K | |
| | VSS | | | |
| MTK-222 | QVQLQQSGADLMKPGASVKL | 25 | DIVLTQSPVSLAVSLGQRATI | 54 |
| | SCKATGYTFTGYWIEWVKQR | | SCRASESVDSYGSSFMHWYQQ | |
| | PGHGLEWIGEILTGSGSSNY | | KPGQPPKLLIYRASSLESGIP | |
| | NEKFKGKATFTADSSSNTAF | | ARFSGSGSRTDFTLTINPVET | |
| | MQLSSLTTEDSAIYYCAGSY | | DDVATYYCQQSNEDPRTFGGG | |
| | YSNSWFPYWGQGSLVTVSA | | TKLEIK | |
| MTK-223 | DVQLQESGPGLVKPSQSLSL | 26 | DVVMTQTPLSLPVSLGDQASI | 55 |
| | TCSVTGYSITSGYYWNWIRQ | | SCRSSQSLVHSDGNTYLHWYL | |
| | FPGNKLEWMGYISYDGTNNY | | QKPGQSPKLLIYKVSNRFSGV | |
| | NPSLKNRISITRDTSENQFF | | PDRFSGSGSGTDFTLKISRVE | |
| | LKLNSVTTEDTATYFCVRGA | | AEDLGVYFCSQSTHVPTWTFG | |
| | GYWGQGTTLTVSS | | GGTKLEIK | |
| MTK-224 | QVQLQQPGAELVKPGASVKV | 27 | DIVMTQSPATLSVTPGDRVSL | 56 |
| | SCKASGYTFTSYWMHWVKQR | | SCRASQIISDYLNWYQQKSHE | |
| | PGQGLEWIGRIHPFDSDTNY | | SPRLLIKYASQSISGIPSRFS | |
| | NQKFKGKATLTVDKSSSTAY | | GSGSGSDFTLSINSVEPEDVG | |
| | MQLSSLTSEDSAVYYCSIAG | | VYYCQNGHSFPLTFGAGTKLE | |
| | FAYWGQGTLVTVSA | | LK | |
| MTK-225 | EVQLQQSGPELVKPGASVKI | 28 | DIQMTQSPASLSVSVGETVTI | 57 |
| | FCKASGYTFTAYNMDWVKQS | | TCRASENIYSSLAWYQQKQGK | |
| | HGKSLEWIGDINPNNGGTIY | | SPQLLVYAATNLADGVPSRFS | |
| | NQKFKGKATLTVDKSSSTAY | | GSGSGTQFYLKINGLQSEDFG | |
| | MELRSLTSEDTAVYYCARRR | | SYYCQHFWGTSYTFGGGTKLE | |
| | RYGNTYYWYFDVWGTGTTVT | | IK | |
| | VSS | | | |
| MTK-226 | QVQLQQSGPELVKPGASVKI | 29 | DAVMTQTPLSLPVSLGDQASI | 58 |
| | SCKASGYSFTSYYIHWVKQR | | SCRSSQSLESSNGNTYLHWYL | |
| | PGQGLEWIGWIFPGNGNTKY | | QKPGQSPQLLIYRVSNRFSGV | |
| | NENFRGKATLTADTSSSTAY | | LDRFSGSGSGTDFTLQISRVE | |
| | MQLSSLTSEDSAVYYCARLG | | AEDLGVYFCLQVTHVPFTFGS | |
| | LIYALDYWGQGTSVTVSS | | GTKLEIK | |
| MTK-227 | EVKLVESGGGLVQPGGSLSL | 30 | DIQMTQSPASLSASVGETVTI | 59 |
| | SCAASGFTFTDNYMTWVRQP | | TCRASENIYSYLAWYQQKQGK | |
| | PGKALEWLGFSRNKANGYTT | | SPQLLVYNAKTLAEGVPSRFS | |
| | EYSASVKGRFTISRDSSQSI | | GSGSGTQFSLKINSLQPADFG | |
| | LYLQMNALRAEDSGTYYCAR | | SYYCQHYYRTPPTFGGGTKLE | |
| | FPPHYYAGGYFDVWGTGTTV | | IK | |
| | TVSS | | | |
| MTK-228 | QDQLQQSGPELVKPGASVKI | 31 | DAVMTQTPLSLPVSLGDQASI | 60 |
| | SCKASGYSFTSYYIHWVKQR | | SCRSSQSLENSNGNTYLHWYL | |
| | PGQGLEWVGWIFPGNGNTKY | | QKPGQSPQLLIYRVSNRFSGV | |
| | NERFKGKATLTADTSSSTAY | | LDRFSGSGSGTDFTLKISRVE | |
| | MQLSSLTSEDSAVYYCARLG | | AEDLGVYFCLQVTHVPFTFGS | |
| | LIYALDYWGQGTSVTVSS | | GTKLEIK | |
| MTK-229 | QVQLQQSGPELVKPGASVKI | 32 | QIVLTQSPAIMSASPGEEVTM | 61 |
| | SCKASGYEFSRSWMNWVKQR | | TCSASSSVSYMHWYQQKSGTS | |
| | PGKGLEWIGRIYPGDGDTNY | | PKRWIYDTSKLASGVSGRFSG | |
| | KGKLKGQATLTADKSSSTAY | | SGSGTSYSLTISSMEAEDAAT | |
| | MQLSSLTFEDSAVYFCARGG | | YYCQQWSSNPPITFGAGTKLE | |
| | GYYYDSRYEGFVYWGQGTVV | | LK | |
| | TVSA | | | |
| MTK-230 | EVQLQQSGPELVKPGASVRI | 33 | DIVLTQSPASLTVSLGQRATI | 62 |
| | PCKASGYTFSDYNMDWVKQS | | SCRASRSVSTSGYSYMHWYQQ | |
| | HGKSLEWIGNINPNIGGTIY | | KPGQPPKLLIYLASNLESGVP | |
| | SQKFKGKATLTVDKSSSTAY | | ARFSGSRSGTDFTLNIHPVEE | |
| | MELRSLTSEDTAVYYCARGD | | EDAATYYCQHSREYPFTFGSG | |
| | YGFASWGQGTLVTVSA | | TKLEIK | |
| MTK-231 | DVQLQESGPGLVKPSQSLSL | 209 | DVVMTQTPLSLPVSLGDQASI | 211 |
| | TCSVTGYSITSGYYWNWIRQ | | SCRSSQSLVHSDGNTYLHWYL | |
| | FPGNKLEWMGYISYDGTYNY | | QKPGQSPKLLIYKVSNRFSGV | |
| | HPSLKNRISITRDTSKNQFF | | PDRFSGSGSGTDFTLKISRLE | |
| | LTLNSVTTEDTATFYCTRGG | | AEDLGIYFCSQTTHIPLTFGS | |
| | GYWGQGTSVTVSS | | GTKLEIK | |
| MTK-232 | EVQLQESGPSLVKPSQTLSL | 210 | DSVMTQSHKFMSTLVGDRVSI | 212 |
| | TCSVTGDSITSGYWNWIRKF | | TCKASQDVGTAVAWYQQKPGQ | |
| | PGNKLEYMGYINYSGSTNYN | | SPKVLIYWASTRHTGVPDRFT | |
| | PSLKSRISITRDTSKNQYYL | | GSGSGTDFTLTITNVQSEDLA | |
| | QLNSVTTEDTATYYCARWNY | | DYFCHQYSTYPYTFGGGTKLE | |
| | LYYFDYWGQGTTLTVSS | | IK | |

The CDR sequences according to Kabat for the anti-MerTK antibodies of the present disclosure are provided below in Table 4 (heavy chain) and Table 5 (light chain).

TABLE 4

| | | SEQ | | SEQ | | SEQ |
|---|---|---|---|---|---|---|
| Antibody | HVR-H1 | ID NO: | HVR-H2 | ID NO: | HVR-H3 | ID NO: |
| MTK-201 | NYGLH | 63 | VIWSGGITD | 81 | KGHDPYAMD | 110 |
| | | | YNAAFIS | | F | |
| MTK-202 | DFGMH | 64 | FISGGSNTI | 82 | NGNSFAY | 111 |
| | | | YYTDTVKG | | | |
| MTK-203 | NYWMH | 65 | RIHPFDTET | 83 | PANGGFAY | 112 |
| | | | NDNQKFKG | | | |
| MTK-204 | DYGMH | 66 | YISSGTSTI | 84 | GGLLFDY | 113 |
| | | | YYEDTVKG | | | |
| MTK-205 | EYIMH | 224 | GINPNNLGI | 225 | DGYFGFDY | 226 |
| | | | IYNQKFKG | | | |
| MTK-206 | SYTMH | 67 | YINPSSGYI | 85 | TVYGYYGSS | 114 |
| | | | KNSQKFKD | | PDY | |
| MTK-207 | SYGVS | 68 | IIWGDGSTN | 86 | GRYGNYEDW | 115 |
| | | | YHSALIS | | YFDV | |
| MTK-208 | NYWMH | 65 | EIDPSDSYA | 87 | CDYYGRLDY | 116 |
| | | | NYNQKFKD | | | |
| MTK-209 | NYWIH | 69 | RIHPFDSEP | 88 | ASFAY | 117 |
| | | | NYNQKFKG | | | |
| MTK-210 | RSWMN | 70 | RIYPGDGDT | 89 | KGGYYYSIS | 118 |
| | | | NYNGKFKG | | NYFDY | |
| MTK-211 | GYWIE | 71 | EILPGSGGS | 90 | GEPSYFSDA | 119 |
| | | | NYNEKFKG | | GFAY | |
| MTK-212 | SSWVN | 72 | RIYPGDGDS | 91 | KSHFIYYTL | 120 |
| | | | NYNGNFKG | | DY | |
| MTK-213 | SYWIT | 73 | EIYPGIGST | 92 | GAYTKYYFD | 121 |
| | | | NYNEKFKS | | Y | |
| MTK-214 | NYWMH | 65 | RIHPFDSDT | 93 | GTLIY | 122 |
| | | | NYNEKFTD | | | |
| MTK-215 | DYGMH | 66 | YISSGSSTI | 94 | GGRYGAMDY | 123 |
| | | | YYGDTVKG | | | |
| MTK-216 | DYGMH | 66 | FISSGSTTI | 95 | GGFHLDY | 124 |
| | | | YYADTVKG | | | |
| MTK-217 | SYWIT | 73 | DIHPGSGIN | 96 | GGYYGSSYY | 125 |
| | | | NYNEKFRS | | VMDY | |
| MTK-218 | SYYIH | 74 | WIFPGNRNT | 97 | LGLIYALDY | 126 |
| | | | QYNEKFKG | | | |
| MTK-219 | GYWIE | 71 | EILTGSGST | 98 | SYFTNSWFP | 127 |
| | | | NYNERFKG | | Y | |
| MTK-220 | DYGMH | 66 | FISGGSGTI | 99 | GGNYFDY | 128 |
| | | | YYEDTVKG | | | |
| MTK-221 | TSGMGVS | 75 | HIYWDDDKR | 100 | LGAYSAFPY | 129 |
| | | | YNTSLQS | | YFDY | |
| MTK-222 | GYWIE | 71 | EILTGSGSS | 101 | SYYSNSWFP | 130 |
| | | | NYNEKFKG | | Y | |
| MTK-223 | SGYYWN | 76 | YISYDGTNN | 102 | GAGY | 131 |
| | | | YNPSLKN | | | |
| MTK-224 | SYWMH | 77 | RIHPFDSDT | 103 | AGFAY | 132 |
| | | | NYNQKFKG | | | |
| MTK-225 | AYNMD | 78 | DINPNNGGT | 104 | RRRYGNTYY | 133 |
| | | | IYNQKFKG | | WYFDV | |
| MTK-226 | SYYIH | 74 | WIFPGNGNT | 105 | LGLIYALDY | 126 |
| | | | KYNENFRG | | | |
| MTK-227 | DNYMT | 79 | FSRNKANGY | 106 | FPPHYYAGG | 134 |
| | | | TTEYSASVK | | YFDV | |
| | | | G | | | |
| MTK-228 | SYYIH | 74 | WIFPGNGNT | 107 | LGLIYALDY | 126 |
| | | | KYNERFKG | | | |
| MTK-229 | RSWMN | 70 | RIYPGDGDT | 108 | GGGYYYDSR | 135 |
| | | | NYKGKLKG | | YEGFVY | |
| MTK-230 | DYNMD | 80 | NINPNIGGT | 109 | GDYGFAS | 136 |
| | | | IYSQKFKG | | | |
| MTK-231 | SGYYWN | 213 | YISYDGTYN | 215 | GGGY | 217 |
| | | | YHPSLKN | | | |
| MTK-232 | SGYWN | 214 | YINYSGSTN | 216 | WNYLYYFDY | 218 |
| | | | YNPSLKS | | | |

TABLE 5

| | | SEQ | | SEQ | | SEQ |
|---|---|---|---|---|---|---|
| Antibody | HVR-L1 | ID NO: | HVR-L2 | ID NO: | HVR-L3 | ID NO: |
| MTK-201 | TASSSVS | 137 | DTSNLAS | 163 | HQYHHSPYT | 185 |
| | SSYLH | | | | | |
| MTK-202 | KSSQSVL | 138 | WASTRES | 164 | HQYFFSWT | 186 |
| | YSSNQKN | | | | | |
| | YLA | | | | | |
| MTK-203 | ITSTDIG | 139 | EGNSLRP | 165 | LQSDNLPLT | 187 |
| | DDMN | | | | | |
| MTK-204 | KSSQSVL | 138 | WASTRES | 164 | HQYFSSWT | 188 |
| | YSSNQKN | | | | | |
| | YLA | | | | | |
| MTK-205 | KASQDVG | 146 | WTSTRRT | 227 | HQYNSYPLT | 228 |
| | TSVA | | | | | |
| MTK-206 | RASQDIG | 140 | ATSSLES | 166 | LQYDSSPWT | 189 |
| | SNLN | | | | | |
| MTK-207 | RASGNIH | 141 | NAKTLAA | 167 | QHFWSIPLT | 190 |
| | NYLA | | | | | |
| MTK-208 | SASSSVS | 142 | STSNLAS | 168 | QQRSSYPPI | 191 |
| | YML | | | | T | |
| MTK-209 | ITSTDID | 143 | AGNTLRP | 169 | LQGGALPLT | 192 |
| | DKMN | | | | | |
| MTK-210 | SASSSVS | 144 | DTSNLAS | 163 | QQWSSYPPY | 193 |
| | YMH | | | | T | |
| MTK-211 | SASSSIT | 145 | DTSKLAS | 170 | HQRSTYPYT | 194 |
| | YMH | | | | | |
| MTK-212 | KASQDVG | 146 | WASSRHT | 171 | QQYSTYPYT | 195 |
| | TSVA | | | | | |
| MTK-213 | KASQDVG | 147 | WASTRHT | 172 | QQYSSYLMY | 196 |
| | DAVA | | | | T | |
| MTK-214 | RASQSIY | 148 | FASDSIS | 173 | LQGYSTPLT | 197 |
| | KKLH | | | | | |
| MTK-215 | RASQSIS | 149 | NPSQSIS | 174 | QQSNSWPFT | 198 |
| | NNLH | | | | | |
| MTK-216 | KSSQSVL | 150 | WASTRES | 164 | HQYFSSWT | 188 |
| | YSANQQN | | | | | |
| | YLA | | | | | |
| MTK-217 | RASQTIG | 151 | YASESIS | 175 | QQSNDWPYT | 199 |
| | TSIH | | | | | |
| MTK-218 | RSSQSLE | 152 | RVSNRFS | 176 | LQVTHVPFT | 200 |
| | NSNGHTY | | | | | |
| | LH | | | | | |
| MTK-219 | RASESVD | 153 | RAYTLES | 177 | QQSNEDPRT | 201 |
| | SYGNSFM | | | | | |
| | H | | | | | |
| MTK-220 | KSSQSVL | 138 | WASTRES | 164 | HQYFSSWT | 188 |
| | YSSNQKN | | | | | |
| | YLA | | | | | |
| MTK-221 | KASQDIH | 154 | YTSTLQP | 178 | LQYDNLWT | 202 |
| | KHIA | | | | | |
| MTK-222 | RASESVD | 155 | RASSLES | 179 | QQSNEDPRT | 201 |
| | SYGSSFM | | | | | |
| | H | | | | | |
| MTK-223 | RSSQSLV | 156 | KVSNRFS | 180 | SQSTHVPTW | 203 |
| | HSDGNTY | | | | T | |
| | LH | | | | | |
| MTK-224 | RASQIIS | 157 | YASQSIS | 181 | QNGHSFPLT | 204 |
| | DYLN | | | | | |
| MTK-225 | RASENIY | 158 | AATNLAD | 182 | QHFWGTSYT | 205 |
| | SSLA | | | | | |
| MTK-226 | RSSQSLE | 159 | RVSNRFS | 176 | LQVTHVPFT | 200 |
| | SSNGNTY | | | | | |
| | LH | | | | | |
| MTK-227 | RASENIY | 160 | NAKTLAE | 183 | QHYYRTPPT | 206 |
| | SYLA | | | | | |
| MTK-228 | RSSQSLE | 161 | RVSNRFS | 176 | LQVTHVPFT | 200 |
| | NSNGNTY | | | | | |
| | LH | | | | | |
| MTK-229 | SASSSVS | 144 | DTSKLAS | 170 | QQWSSNPPI | 207 |
| | YMH | | | | T | |
| MTK-230 | RASRSVS | 162 | LASNLES | 184 | QHSREYPFT | 208 |
| | TSGYSYM | | | | | |
| | H | | | | | |
| MTK-231 | RSSQSLV | 156 | KVSNRFS | 180 | SQTTHIPLT | 219 |
| | HSDGNTY | | | | | |
| | LH | | | | | |
| MTK-232 | KASQDVG | 220 | WASTRHT | 172 | HQYSTYPYT | 221 |
| | TAVA | | | | | |

### Example 9: Production of Anti-MerTK Antibodies

Anti-MerTK hybridoma clones were cultured in serum free hybridoma media, and the anti-MerTK antibodies in the supernatants were purified on Hamilton STAR platform (Hamilton Company, Reno, NV) using Protein A tips (Phynexus Inc, San Jose, CA). Anti-MerTK antibodies were also produced via direct cloning of the variable gene regions obtained from the hybridomas into a recombinant expression plasmid for production of chimeric antibodies containing a human IgG1 Fc domain. Using the Tuna293™ and TunaCHO™ Processes (LakePharma, San Carlos, CA), proprietary HEK293 (Tuna293™) or CHO-K1 (TunaCHO™) derived cells were seeded into shake flasks and expanded using serum-free chemically defined media. The expression plasmids were transiently transfected into the cells and the culture supernatants were harvested 7 and 14 days later. After clarification by centrifugation and filtration, the anti-MerTK antibodies in the supernatants were purified via Protein A chromatography.

### Example 10: Anti-MerTK Antibodies Bind to SK-MEL-5 Cells, CHO-muMerTK OE Cells, and Mouse Macrophages

To determine whether recombinant anti-human MerTK antibodies of the present disclosure bind to human MerTK endogenously expressed on SK-MEL-5 cells and CHO cells overexpressing mouse MerTK (CHO-muMerTK OE cells), as compared to A375 and parental CHO cells as negative controls, the following experiments were performed. SK-MEL-5 cells, A375 cells, CHO-muMerTK OE cells, and CHO parental cells were plated at 100,000 cells/well. Anti-MerTK antibodies were added to the cells at 10 μg/ml. After 60 minutes on ice, cells were washed and then stained with PE-conjugated goat anti-human IgG antibody (Southern Biotech Cat #2040-09, Birmingham, AL) in the presence of Fc block solution on ice for 30 minutes, and then washed twice with cold FACS buffer (2% FBS in PBS). Mouse anti-human MerTK-PE conjugated (Biolegend, Clone 590H11G1E3) or anti-mouse MerTK-PE conjugated (ThermoFisher, Clone DS5MMER) antibodies were used as positive controls. Stained cells were acquired on a BD FACS Canto II cytometer and the mean fluorescence intensity (MFI) was calculated with FlowJo.

To confirm anti-MerTK antibody binding to endogenous murine MerTK, bone marrow-derived macrophages (BMDM) were isolated from MerTK wild type (WT) and knockout (KO) mice (The Jackson Laboratory, Cat #011122) using standard protocols. Briefly, cells isolated from the bone marrow were plated in the presence of 50 ng/ml M-CSF (R&D Systems, Cat #416-ML) for seven days to allow for differentiation into macrophages. Cell staining, FACs, and MFI were performed and calculated as described above.

Table 6 below shows MFI values from FACS analysis of anti-MerTK antibodies binding to SK-MEL-5 cells, A375 cells, CHO-muMerTK-OE cells, CHO parental cells, and BMDM cells from MerTK WT mice (with the extent of binding/MFI obtained with BMDM cells from MerTK KO mice subtracted from that obtained from MerTK WT mice).

TABLE 6

| | | | CHO- | CHO | BMDM |
|---|---|---|---|---|---|
| Antibody | SK-MEL-5 | A375 | muMerTK OE | parentals | WT-KO |
| MTK-201 | 40345 | 519 | 252 | 341 | 1033 |
| MTK-202 | 46084 | 377 | 4407 | 254 | 4513 |
| MTK-203 | 24624 | 308 | 2083 | 775 | 2985 |
| MTK-204 | 33759 | 329 | 713 | 285 | 602 |
| MTK-205 | 31489 | 313 | 240 | 232 | 737 |
| MTK-206 | 3266 | 318 | 286 | 254 | 431 |
| MTK-207 | 1188 | 336 | 279 | 253 | 989 |
| MTK-208 | 14054 | 331 | 329 | 275 | 1173 |
| MTK-209 | 46307 | 344 | 1220 | 769 | 813 |
| MTK-210 | 29944 | 331 | 252 | 244 | 1271 |
| MTK-211 | 30523 | 331 | 5158 | 510 | 4492 |
| MTK-212 | 8081 | 363 | 12845 | 728 | 9094 |
| MTK-213 | 40345 | 321 | 12815 | 750 | 8447 |
| MTK-214 | 58179 | 558 | n.a. | n.a. | 2949 |
| MTK-215 | 19031 | 308 | 237 | 242 | 466 |
| MTK-216 | 43273 | 333 | n.a. | n.a. | 1031 |
| MTK-217 | 52405 | 313 | n.a. | n.a. | 1133 |
| MTK-218 | 48962 | 347 | 13204 | 717 | 11766 |
| MTK-219 | 26828 | 306 | 11403 | 619 | 6176 |
| MTK-220 | 28958 | 347 | 5799 | 497 | 757 |
| MTK-221 | 42753 | 322 | 374 | 252 | 993 |
| MTK-222 | 27476 | 308 | 4761 | 396 | 3682 |
| MTK-223 | 26764 | 312 | 234 | 234 | 316 |
| MTK-224 | 45309 | 362 | 616 | 398 | 1864 |
| MTK-225 | 40247 | 316 | 285 | 252 | 619 |
| MTK-226 | 49081 | 325 | 11534 | 810 | 10446 |
| MTK-227 | 40933 | 318 | 14184 | 603 | 5016 |
| MTK-228 | 60196 | 333 | 17449 | 933 | 7371 |
| MTK-229 | 32176 | 340 | 2002 | 341 | 2873 |
| MTK-230 | 42444 | 331 | 12353 | 769 | 9121 |
| MTK-231 | 39004 | 369 | 594 | 594 | 639 |
| MTK-232 | 10041 | 306 | 297 | 297 | 412 |
| H1 | 794 | 297 | | | |
| DS5MMER | | | 3497 | 219 | 2798 |

n.a. data not available

The recombinant anti-MerTK antibodies bound to SK-MEL-5 cells, indicating anti-MerTK antibody binding to human MerTK. Twelve recombinant anti-MerTK antibodies showed strong to moderate binding of CHO-muMerTK overexpressing cells and BMDMs, indicating anti-MerTK antibody binding to mouse MerTK.

### Example 11: Anti-MerTK Antibody Blocking of Ligand Gas6 and Ligand ProS Binding to MerTK

To identify anti-MerTK antibodies that do not inhibit the ability of Gas6 and/or ProS ligand binding to human MerTK, ligand blocking assays were performed as described above. Anti-MerTK antibodies were titrated to a final concentration from 66.6 nM to 4 pM and tested against anti-human MerTK antibody M6 (disclosed in WO2016/106221) or isotype control antibody.

Table 7 shows Gas6 binding and ProS binding to human MerTK at the highest concentration (66.6 nM) of antibody tested in these studies; data is presented as a percentage of Gas6 binding to MerTK and percentage of ProS binding to human MerTK compared to the extent of ligand binding of isotype control huIgG1 antibody, which was set to 100% binding.

Anti-MerTK antibodies of the present disclosure did not inhibit (i.e., did not block) Gas6 binding to human MerTK, as shown below in Table 7. The majority of anti-MerTK antibodies of the present disclosure did not block ProS ligand binding to human MerTK. Additionally, anti-MerTK antibodies MTK-202, MTK-212, MTK-213, MTK-220, MTK-223, and MTK-230 displayed a modest inhibition (>30% inhibition) of ligand binding to MerTK in this assay. As shown, anti-MerTK antibody M6 was very effective at blocking binding of both Gas6 and ProS to human MerTK, which blocked ligand binding by more than 90%. Also as shown in Table 7, certain anti-MerTK antibodies of the present disclosure appeared to enhance binding of Gas 6 and/or ProS to MerTK.

TABLE 7

| | Percent Gas6 | Percent ProS |
|---|---|---|
| Antibody | ligand binding | ligand binding |
| MTK-201 | 112.4 | 94.3 |
| MTK-202 | 100.6 | 53.4 |
| MTK-203 | 103.7 | 79.7 |
| MTK-204 | 120.8 | 101.4 |
| MTK-205 | 104.9 | 70.3 |
| MTK-206 | 103.0 | 131.6 |
| MTK-207 | 115.7 | 95.5 |
| MTK-208 | 107.2 | 71.7 |
| MTK-209 | 98.9 | 82.7 |
| MTK-210 | 113.5 | 116.2 |
| MTK-211 | 115.1 | 106.4 |
| MTK-212 | 122.0 | 45.8 |
| MTK-213 | 102.1 | 43.2 |
| MTK-214 | 109.8 | 92.1 |
| MTK-215 | 110.1 | 81.6 |
| MTK-216 | 104.1 | 72.8 |
| MTK-217 | 107.4 | 112.8 |
| MTK-218 | 112.2 | 179.4 |
| MTK-219 | 106.4 | 100.0 |
| MTK-220 | 97.0 | 63.4 |
| MTK-221 | 106.0 | 72.8 |
| MTK-222 | 104.7 | 88.2 |
| MTK-223 | 106.7 | 37.4 |
| MTK-224 | 105.9 | 86.8 |
| MTK-225 | 119.8 | 185.7 |
| MTK-226 | 108.8 | 174.7 |
| MTK-227 | 99.9 | 84.5 |
| MTK-228 | 110.2 | 216.3 |
| MTK-229 | 98.9 | 117.0 |
| MTK-230 | 105.9 | 57.6 |
| MTK-231 | 116.0 | 152.1 |
| MTK-232 | 92.4 | 71.7 |
| Isotype control | 100.0 | 100.0 |
| M6 | 1.2 | 5.3 |

### Example 12: Direct Anti-MerTK Antibody Binding of Gas6 and ProS Ligands

To identify anti-MerTK antibodies that directly bind Gas6 or ProS, binding assays were performed as follows. Briefly, rabbit anti-human IgG antibody (Jackson ImmunoResearch, Cat #309-005-008) was coated at 2 μg/ml onto high-protein binding plates at 4° C. overnight. After washing with 0.05% Tween20 in PBS three times, 5% BSA in PBS was added for 1 hour. Recombinant human MerTK-human Fc Chimera protein (R&D systems, Cat #391-MR-100) or anti-MerTK antibodies of the present disclosure were added at 2 μg/ml for 1 hour and plates were washed before the addition of His tag-conjugated recombinant Gas6 (R&D systems, Cat #885-GSB-050) at 1.5 μg/ml or His tag-conjugated recombinant ProS (R&D systems, Cat #9489-PS-100) at 10 μg/ml. After a further incubation for 1 hour, plates were washed and incubated for 1 hour with HRP-conjugated anti-6×His tagged antibody (Abcam, Cambridge, MA, Cat #ab1187). Plates were then washed and an HRP substrate, TMB, was added to develop the plates. The reaction was stopped by adding 50 μl 2N H₂SO₄ and the OD was measured using a spectrophotometer (BioTek).

Of the 32 antibodies tested for their ability to bind either or both Gas6 and ProS ligands in the absence of MerTK, only anti-MerTK antibody MTK-231 was able to strongly bind both ligands. Additionally, anti-MerTK antibodies MTK-207, MTK-211, MTK-213, MTK-214, MTK-224, and MTK-232 showed little to moderate binding to ProS ligand. These results indicated that certain anti-MerTK antibodies of the present disclosure have the additional property of binding both MerTK and one or more of its ligands.

### Example 13: Modulating MerTK Tyrosine Phosphorylation

The effect of anti-MerTK antibodies on MerTK tyrosine phosphorylation (p-MerTK) in myeloid cells was examined as follows. To generate monocyte-derived macrophages, human primary monocytes were isolated from heparinized human blood (Blood Centers of the Pacific) using RosetteSep Human Monocyte Enrichment Cocktail (STEMCELL Technologies), according to the manufacturer's protocol. Monocytes were seeded in RPMI (Invitrogen) containing 10% Fetal Bovine Serum (Hyclone) and 50 ng/mL M-CSF (Biolegend) to induce differentiation of macrophages. After 6 days, macrophages were harvested by removing media, incubating with 3 mM EDTA for 5 min at 37° C. and subsequently scraping cells. Macrophages were then plated on 96-well plates at 0.1×10⁶/well and cultured with 50 ng/mL M-CSF (Biolegend), 100 nM Dexamethasone (Tocris), 50 ng/mL human Tgfβ (R&D Systems) and 20 ng/mL of IL-10 (Pepro Tech). Two days later (˜48 hours), cells were treated with anti-MerTK antibodies by removing media from cells and adding anti-MerTK antibodies in PBS at 10 μg/mL and incubating cells for 8 min at 37° C. Cells were then harvested by removing treatment antibodies, washing cells once with ice-cold PBS, and lysing cells with 150 μL of ice-cold 1× lysis buffer (Cell Signaling Technology). 96-well plates were incubated in lysis buffer for 30 min on shaker at 4° C. Plates were then cleared of cellular debris by centrifugation at 4,300×g for 10 min at 4° C. Supernatant was collected for phospho-Mer (panTyr) ELISA (Cell Signaling Technology) and BCA (ThermoFisher Scientific). Lysates were subsequently processed according to manufacturer's instructions and p-MerTK levels were normalized to total protein levels.

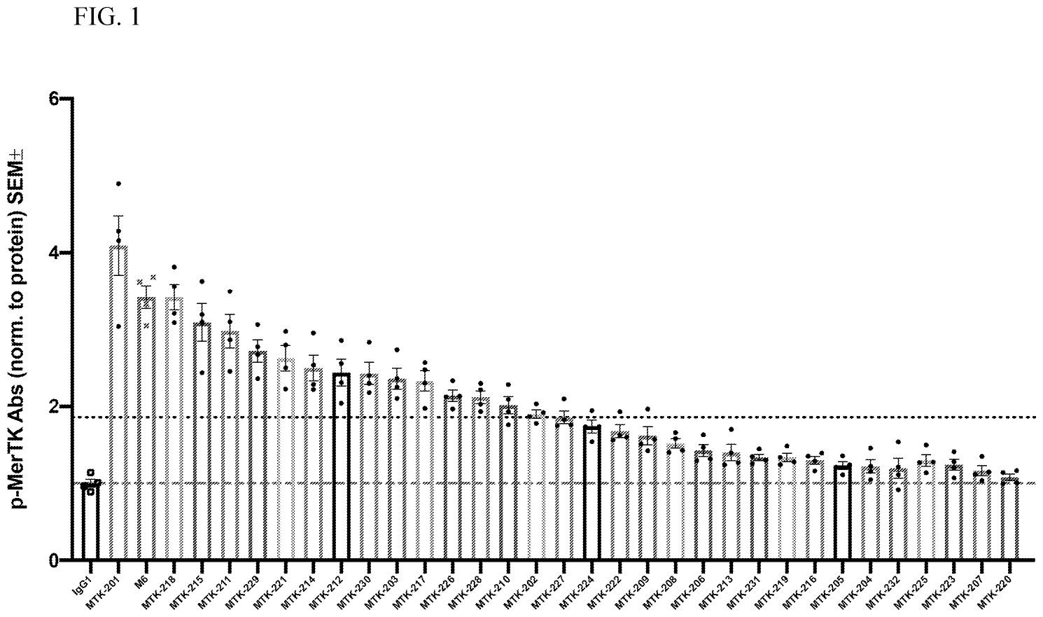

Many of the anti-MerTK antibodies of the present disclosure were able to induce p-MerTK as tested in this assay (See FIG. 1). Anti-MerTK antibodies showing a significant increase in p-MerTK levels (compared to that observed in isotype control huIgG1 antibody treated cells, as indicated by the lower dashed line in FIG. 1), using a one-way ANOVA Tukey's multiple comparison test) at or above the standard error of the mean (indicated by the upper dotted line in FIG. 1). Isotype control huIgG1 antibody was set to a value of 1. N=4 with each N being one human donor.

These results showed that anti-MerTK antibodies of the present disclosure agonize MerTK activity, at least in part, by inducing or increasing phosphorylation of MerTK.

### Example 14: Binding Kinetics of Anti-MerTK Antibodies

Binding kinetics of anti-MerTK IgG1 antibodies of the present disclosure to human, cyno, and murine MerTK were evaluated using a Carterra LSA instrument (Carterra, Salt Lake City, UT). Briefly, anti-MerTK antibodies were prepared by diluting 50-, 250- and 500-fold into 10 mM Acetate, pH 4.25 (Carterra), to give final concentrations ranging from 1 to 113 μg/ml. A HC200M sensor chip (Carterra) was activated using the single channel flow cell with a 7-minute injection of a 1:1:1 mixture of 100 mM MES pH 5.5, 100 mM sulfo-NHS, 400 mM EDC (all reconstituted in MES pH 5.5; 100 μl of each mixed in vial immediately before running assay). After switching to the multi-channel array flow cell, the antibodies were injected over the activated chip in three 96-spot arrays for 10 minutes each. The remaining unconjugated active groups on the chip were then blocked by injecting 1M Ethanolamine pH 8.5 (Carterra) for 7 minutes using the single channel flow cell. The resulting sensor chip contained three spots for each antibody, at three different densities. Two independent experiments were performed as follows, resulting in an N of between one and six determinations for each antibody. Spots that yielded less than 25 RU of analyte binding were excluded from further analysis.

After priming with running buffer (HBS-TE, Carterra) with 0.5 mg/ml BSA (Sigma), the immobilized anti-MerTK antibodies were tested for their ability to bind to several forms of recombinant MerTK extracellular domain, including human, cynomolgus, and mouse orthologs as described above. Estimates of affinity were generated by injecting each analyte over the entire antibody array using the single channel flow cell. MerTK analytes were diluted with running buffer, in a series of six, three-fold serial dilutions starting from 1 μM for human and cynomolgus MerTK, and 600 nM for mouse MerTK. Analytes were injected for 5 minutes, and dissociation was followed for 10 minutes. After each analyte injection, antibodies were regenerated with either Protein A/G Elution Buffer (Thermo) with 1M NaCl (Teknova), or with 10 mM Glycine pH 2.5. Three buffer blanks were run between each series (one species per series). Data were processed and analyzed using NextGenKIT high-throughput kinetics analysis software (Carterra).

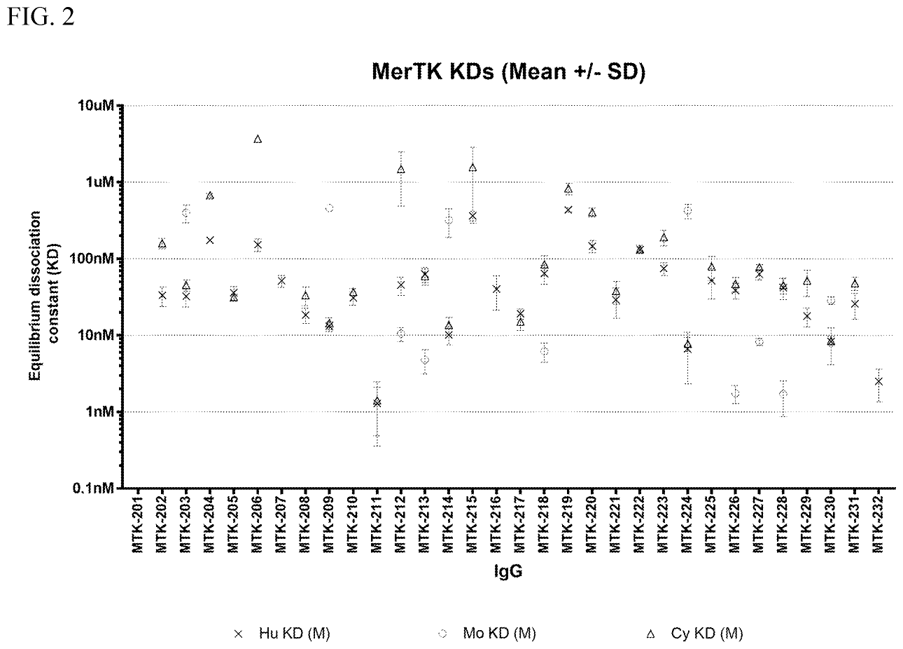

The equilibrium dissociation constants (K_{D}) were calculated from the fitted association and dissociation rate constants (k-on and k-off, respectively) for each of the anti-MerTK antibodies tested. The values were combined, means and standard deviation calculated, and graphs prepared using GraphPad Prism. The K_{D} values are summarized in Table 8 below. Also, see FIG. 2.

TABLE 8

| | Human MerTK | | | Cynomolgus MerTK | | | Mouse MerTK | | |
|---|---|---|---|---|---|---|---|---|---|
| | Mean | | | Mean | | | Mean | | |
| Antibody | KD (M) | SD | N | KD (M) | SD | N | KD (M) | SD | N |
| MTK-201 | NT | NA | NA | NT | NA | NA | NT | NA | NA |
| MTK-202 | 3.3E−08 | 9.6E−09 | 5 | 1.6E−07 | 2.4E−08 | 4 | LB | NA | NA |
| MTK-203 | 3.2E−08 | 8.8E−09 | 6 | 4.6E−08 | 7.6E−09 | 6 | 4.0E−07 | 1.0E−07 | 4 |
| MTK-204 | 2.4E−07 | 1.0E−07 | 6 | 6.8E−07 | 3.5E−08 | 2 | LB | NA | NA |
| MTK-205 | 3.6E−08 | 7.3E−09 | 6 | 3.2E−08 | 3.0E−09 | 6 | LB | NA | NA |
| MTK-206 | 1.5E−07 | 2.9E−08 | 6 | 3.7E−06 | NA | 1 | NB | NA | NA |
| MTK-207 | 5.2E−08 | 9.2E−09 | 2 | LB | NA | NA | LB | NA | NA |
| MTK-208 | 1.9E−08 | 4.1E−09 | 4 | 3.4E−08 | 9.2E−09 | 2 | NB | NA | NA |
| MTK-209 | 1.3E−08 | 1.9E−09 | 6 | 1.4E−08 | 2.5E−09 | 6 | 4.6E−07 | NA | 1 |
| MTK-210 | 3.1E−08 | 6.2E−09 | 6 | 3.7E−08 | 3.9E−09 | 6 | NB | NA | NA |
| MTK-211 | 1.3E−09 | 8.0E−10 | 6 | 1.4E−09 | 1.1E−09 | 6 | NB | NA | NA |
| MTK-212 | 8.2E−08 | 5.7E−08 | 6 | 1.5E−06 | 1.0E−06 | 2 | 1.1E−08 | 2.2E−09 | 6 |
| MTK-213 | 6.4E−08 | 1.4E−08 | 6 | 6.0E−08 | 1.4E−08 | 6 | 4.8E−09 | 1.7E−09 | 6 |
| MTK-214 | 1.0E−08 | 2.7E−09 | 6 | 1.4E−08 | 3.5E−09 | 6 | 3.2E−07 | 1.3E−07 | 4 |
| MTK-215 | 3.7E−07 | 5.2E−08 | 4 | 1.6E−06 | 1.3E−06 | 3 | NB | NA | NA |
| MTK-216 | 4.1E−08 | 1.9E−08 | 4 | LB | NA | NA | LB | NA | NA |
| MTK-217 | 1.9E−08 | 2.9E−09 | 6 | 1.5E−08 | 3.5E−09 | 6 | NB | NA | NA |
| MTK-218 | 6.4E−08 | 1.8E−08 | 6 | 8.5E−08 | 2.5E−08 | 6 | 6.2E−09 | 1.7E−09 | 6 |
| MTK-219 | 4.4E−07 | 2.9E−08 | 4 | 8.3E−07 | 1.4E−07 | 4 | NB | NA | NA |
| MTK-220 | 1.5E−07 | 2.7E−08 | 6 | 4.1E−07 | 5.4E−08 | 6 | LB | NA | NA |
| MTK-221 | 2.9E−08 | 1.3E−08 | 6 | 3.8E−08 | 1.3E−08 | 6 | NB | NA | NA |
| MTK-222 | 1.3E−07 | 1.2E−08 | 6 | 1.3E−07 | 1.6E−08 | 6 | LB | NA | NA |
| MTK-223 | 7.5E−08 | 1.4E−08 | 6 | 1.9E−07 | 4.4E−08 | 6 | LB | NA | NA |
| MTK-224 | 6.7E−09 | 4.3E−09 | 6 | 7.8E−09 | 1.6E−09 | 6 | 4.3E−07 | 9.3E−08 | 4 |
| MTK-225 | 5.2E−08 | 2.2E−08 | 6 | 8.0E−08 | 2.8E−08 | 6 | LB | NA | NA |
| MTK-226 | 3.9E−08 | 9.0E−09 | 6 | 4.7E−08 | 1.0E−08 | 6 | 1.8E−09 | 4.6E−10 | 6 |
| MTK-227 | 9.9E−08 | 5.7E−08 | 6 | 7.8E−08 | 6.2E−09 | 4 | 8.3E−09 | 8.5E−10 | 6 |
| MTK-228 | 4.2E−08 | 1.3E−08 | 6 | 4.6E−08 | 1.1E−08 | 6 | 1.7E−09 | 8.3E−10 | 6 |
| MTK-229 | 1.8E−08 | 5.0E−09 | 6 | 5.2E−08 | 2.0E−08 | 6 | LB | NA | NA |
| MTK-230 | 8.3E−09 | 4.2E−09 | 6 | 8.5E−09 | 1.2E−09 | 6 | 2.9E−08 | 3.3E−09 | 6 |
| MTK-231 | 2.6E−08 | 9.5E−09 | 6 | 4.8E−08 | 9.5E−09 | 6 | LB | NA | NA |
| MTK-232 | 2.5E−09 | 1.1E−09 | 3 | LB | NA | NA | NB | NA | NA |

N = number of determinations; NT = not tested; NA = not applicable; LB = low binding (below limit of measurement); NB = no binding detected.

These results illustrated that anti-MerTK antibodies of the present disclosure exhibited a range of binding affinities to MerTK of approximately 1 nM to 4 μM. Additionally, these results showed that anti-MerTK antibodies of the present disclosure displayed a range of species binding specificity within this binding affinity range, including human-specific, human and cynomolgus cross-reactive only, or human, cynomolgus, and mouse cross-reactive. In particular, affinity of anti-MerTK antibodies of the present disclosure for binding to human MerTK ranged from approximately 1.3 nM to 440 nM; affinity of anti-MerTK antibodies of the present disclosure for binding to cynomolgus MerTK ranged from 1.4 nM to 3.6 μM; and affinity of anti-MerTK antibodies the present disclosure for binding to murine MerTK ranged from 1.7 nM to 460 nM.

### Example 15: Cross-Reactivity of Anti-MerTK Antibodies to Human, Cyno, and Mouse MerTK

Species cross-reactivity of anti-MerTK antibodies of the present disclosure was determined from the binding kinetic analysis data described above. The results of species binding cross-reactivity analysis of anti-MerTK antibodies of the present disclosure are summarized below in Table 9.

TABLE 9

| Human MerTK | | |
|---|---|---|
| Strong | Human-Cyno MerTK | Human-Cyno-Mouse MerTK |
| preference | Cross Reactive | Cross Reactive |
| MTK-207 | MTK-202, MTK-204, MTK-205, | MTK-203, MTK-209, MTK-212, |
| MTK-216 | MTK-206, MTK-208, MTK-210, | MTK-213, MTK-214, MTK-218, |
| MTK-232 | MTK-211, MTK-215, MTK-217, | MTK-224, MTK-226, MTK-227, |
| | MTK-219, MTK-220, MTK-221, | MTK-228, MTK-230 |
| | MTK-222, MTK-223, MTK-225, | |
| | MTK-229, MTK-231 | |

The relative strength of anti-MerTK antibody binding to cynomolgus and mouse MerTK compared to that observed to human MerTK is summarized in Table 10 below. Note that a higher KD value represents a lower binding affinity.

TABLE 10

| | Ratio of KD | Ratio of KD |
|---|---|---|
| | for Cynomolgus vs | for Mouse vs |
| Antibody | Human MerTK | Human MerTK |
| MTK-201 | NT | NT |
| MTK-202 | 4.8 | LB |
| MTK-203 | 1.4 | 12.5 |
| MTK-204 | 2.8 | LB |
| MTK-205 | 0.9 | LB |
| MTK-206 | 24.7 | NB |
| MTK-207 | LB | LB |
| MTK-208 | 1.8 | NB |
| MTK-209 | 1.1 | 35.4 |
| MTK-210 | 1.2 | NB |
| MTK-211 | 1.1 | NB |
| MTK-212 | 18.3 | 0.13 |
| MTK-213 | 0.9 | 0.08 |
| MTK-214 | 1.4 | 32.0 |
| MTK-215 | 4.3 | NB |
| MTK-216 | LB | LB |
| MTK-217 | 0.8 | NB |
| MTK-218 | 1.3 | 0.10 |
| MTK-219 | 1.9 | NB |
| MTK-220 | 2.7 | LB |
| MTK-221 | 1.3 | NB |
| MTK-222 | 1.0 | LB |
| MTK-223 | 2.5 | LB |
| MTK-224 | 1.2 | 64.2 |
| MTK-225 | 1.5 | LB |
| MTK-226 | 1.2 | 0.05 |
| MTK-227 | 0.8 | 0.08 |
| MTK-228 | 1.1 | 0.04 |
| MTK-229 | 2.9 | LB |
| MTK-230 | 1.0 | 3.5 |
| MTK-231 | 1.8 | LB |
| MTK-232 | LB | NB |

NT = not tested;
LB = low binding (below limit of measurement);
NB = no binding detected

The results from these binding experiments showed that three anti-MerTK antibodies of the present disclosure displayed a strong preference for binding to human MerTK only (and did not show binding to cynomolgus or mouse MerTK in a quantifiable range); these were anti-MerTK antibodies MTK-207, MTK-216, and MTK-232.

The majority of anti-MerTK antibodies of the present disclosure showed binding cross-reactivity to both human and cynomolgus MerTK; these were anti-MerTK antibodies MTK-202, MTK-204, MTK-205, MTK-206, MTK-208, MTK-210, MTK-211, MTK-215, MTK-217, MTK-219, MTK-220, MTK-221, MTK-222, MTK-223, MTK-225, MTK-229, and MTK-231. Of these anti-MerTK antibodies, six showed weak preference of 2- of 5-fold for reactivity to human MerTK over reactivity to cynomolgus MerTK (anti-MerTK antibodies MTK-202, MTK-204, MTK-215, MTK-220, MTK-223, and MTK-229), and two showed moderate preference of 24- and 18-fold for reactivity to human MerTK over reactivity to cynomolgus MerTK (anti-MerTK MTK-206 and MTK-212, respectively).

Eleven anti-MerTK antibodies of the present disclosure displayed cross-reactivity to all three species tested (human, cynomolgus, and mouse); there were anti-MerTK antibodies MerTK (MTK-203, MTK-209, MTK-212, MTK-213, MTK-214, MTK-218, MTK-224, MTK-226, MTK-227, MTK-228, and MTK-230. Of the mouse cross-reactive antibodies, one showed weak preference of 3.5-fold for human MerTK relative to mouse MerTK (anti-MerTK antibody MTK-230) and three showed moderate preference for human over mouse MerTK binding of 12.5-, 35.4-, and 64.2-fold (anti-MerTK antibodies MTK-203, MTK-209, and MTK-224). Six antibodies showed moderate preference for mouse MerTK relative to human MerTK with a range of relative binding of 5- to 30-fold (anti-MerTK antibodies MTK-212, MTK-213, MTK-218, MTK-226, MTK-227, and MTK-228).

### Example 16: Epitope Binning Analysis of Anti-MerTK Antibodies

Epitope binning analysis was performed on anti-MerTK antibodies of the present disclosure by performing tandem injection experiments using a Carterra LSA instrument (Carterra, Salt Lake City, UT). Briefly, the chip used for the kinetic evaluations described above was then tested in a binning assay, in which the immobilized antibodies were tested for their ability to form sandwich pairs with recombinant human MerTK extracellular domain and injected antibodies. For each cycle, 100 nM MerTK was injected over the chip for 5 minutes, followed by a test antibody (diluted to 30 μg/ml in running buffer) for 5 minutes, then by two 30-second injections of 10 mM Glycine pH2.5 (Carterra) for regeneration. Data were processed and analyzed using Epitope high-throughput binning analysis software (Carterra). Antibodies which were able to bind antigen captured by an immobilized antibody were designated as “sandwich” or “pairing” antibodies, and these antibodies were assigned into a different epitope bin from that of the immobilized antibody. A matrix of pairing and non-pairing antibodies was constructed from the binding results of these experiments, which allowed for an epitope bin landscape of the anti-MerTK antibodies to be generated. Some of antibodies have overlapping binning profiles suggesting that they may recognize adjacent, but not completely overlapping epitopes. Slight heterogeneity within bins are indicated by adding letters a, b, c, and d to the bin designations. For antibodies that appear to overlap two epitope bins, the bin numbers are designated with an underscore to indicate the overlapping bin. The epitope bins as determined by this assay are summarized in Table 11 below.

TABLE 11

| Bin | Sample ID |
|---|---|
| 1a | MTK-201 |
| 1 | MTK-211, MTK-217 |
| 1_2 | MTK-208 |
| 2 | MTK-210, MTK-229 |
| 3a | MTK-221 |
| 3b | MTK-222, MTK-227 |
| 3c | MTK-219 |
| 3_4 | MTK-205 |
| 4a | MTK-203, MTK-209 |
| 4b | MTK-230 |
| 4c | MTK-214, MTK-224 |
| 5 | MTK-212 |
| 6 | MTK-215 |
| 7 | MTK-213, MTK-218, MTK-228, MTK-226 |
| 7_8 | MTK-232 |
| 8 | MTK-225 |
| 9a | MTK-202, MTK-204, MTK-216, MTK-220 |
| 9b | MTK-231, MTK-223 |
| 10 | MTK-206, MTK-207 |

As part of the epitope binning experiments described herein, additional studies were performed to investigate the ability of Gas6 and ProS to bind to MerTK captured by the immobilized anti-MerTK antibodies. Anti-MerTK antibodies that do not block binding of either of these two ligands are expected to show sandwich pairing, while anti-MerTK antibodies that block Gas6 and/or ProS would not form binding pairs. The results of this analysis are shown in Table 12 below.

Twenty-two anti-MerTK antibodies of the present disclosure paired with Gas6 but did not pair with ProS assessed by this assay. These results were consistent with experimental results described above showing anti-MerTK antibodies that were ProS only blockers; MTK-202, MTK-203, MTK-205, MTK-206, MTK-207, MTK-213, MTK-214, MTK-215, MTK-218, MTK-219, MTK-220, MTK-221, MTK-222, MTK-223, MTK-224, MTK-225, MTK-227, MTK-228, MTK-229, MTK-230, and MTK-232. Four anti-MerTK antibodies paired with both Gas6 and ProS, which is consistent with non-blockers (MTK-208, MTK-212, MTK-216, and MTK-231). Five anti-MerTK antibodies paired with neither Gas6 nor ProS, which is consistent with double-blockers (MTK-209, MTK-210, MTK-211, MTK-217, and MTK-226). It should be noted that the lack of pairing of an antibody with Gas6 or ProS is suggestive, but may not completely predict, functional blocking as this property will also depend on the relative kinetics and concentrations of the antibody and the ligand for binding to the native receptors on cells.

TABLE 12

| | | Pairs with neither |
|---|---|---|
| Pairs with Gas6 but not | Pairs with both Gas6 | Gas6 nor ProS |
| with ProS (consistent | and ProS (consistent | (consistent with |
| with ProS blockers) | with non-blockers) | double-blockers) |
| MTK-202, MTK-203, MTK-204, | MTK-208, MTK-212, | MTK-209, MTK-210, |
| MTK-205, MTK-206, MTK-207, | MTK-216, MTK-231 | MTK-211, MTK-217, |
| MTK-213, MTK-214, MTK-215, | | MTK-226 |
| MTK-218, MTK-219, MTK-220, | | |
| MTK-221, MTK-222, MTK-223, | | |
| MTK-224, MTK-225, MTK-227, | | |
| MTK-228, MTK-229, MTK-230, | | |
| MTK-232 | | |

### Example 17: Effect of Anti-MerTK Antibodies on MCP-1 Production

Monocyte Chemoattractant Protein-1 (MCP-1) recruits immune cells to sites of injury. In the mouse EAE model of demyelination, deficiency of MCP-1 protects mice from disease progression as inflammatory immune cells are not recruited to the site of injury due to lack of MCP-1 and related inflammatory response.

The effect of anti-MerTK antibodies of the present disclosure on the production of the chemokine MCP-1 was determined as follows. Human primary monocytes were isolated from peripheral blood mononuclear cells using Human Monocyte Isolation Kit (STEMCELL Technologies), according to the manufacturer's instructions. The monocytes were differentiated for 6 days to macrophages in RPMI1640 (Gibco) supplemented with 2% Hepes (Life technologies), 2% Glutamax (Life technologies), 2% penicillin/streptomycin (Life technologies), 2% sodium pyruvate (Life technologies), 2% non-essential amino acids (Life technologies), 10% heat-inactivated fetal bovine serum (HyClone), 8% human serum AB (Sigma), and 50 ng/ml macrophage colony-stimulating factor (M-CSF, R&D Systems). The macrophages were collected by removing media, washing with PBS, then incubating in PBS containing 3 mM EDTA for 5 min at 37° C. before scraping the cells for their removal. The cells were counted and seeded in 96-well flat bottom culture dishes at 50,000 cells per well in 100 μl media. The cells were polarized to M2c macrophage phenotype in the same basal media as for the differentiation with the exception of serum and M-CSF which were omitted. That basal media was supplemented with 50 ng/ml transforming growth factor beta (TGF-beta, Peprotech), 20 ng/ml interleukin-10 (IL-10, Peprotech), 100 nM dexamethasone (Tocris), and anti-MerTK antibodies (10 μg/ml) for two days. In some instances, the cells were incubated in the presence of MerTK ligand ProS (100 nM recombinant human Protein S) during polarization.

Apoptotic cells can activate MerTK expressed on M2c macrophages by its interaction with endogenous ligands associated with the apoptotic cells (e.g., Gas6, ProS). In parallel studies designed to examine the effects of anti-MerTK antibodies of the present disclosure on increasing MCP-1 levels in M2c cells concurrently activated via the presence of apoptotic cells, macrophages were polarized to M2c macrophage phenotype in the presence of apoptotic Jurkat cells generated by overnight culture in RPMI1640 (Gibco) supplemented with 10% fetal bovine serum (HyClone), 1% penicillin/streptomycin (Life technologies), and 0.5 μM staurosporine (R&D Systems). After two days, cell supernatants were collected. MCP-1 concentration in the supernatants was determined using U-PLEX Human MCP-1 Assay (MesoScale Diagnostics).

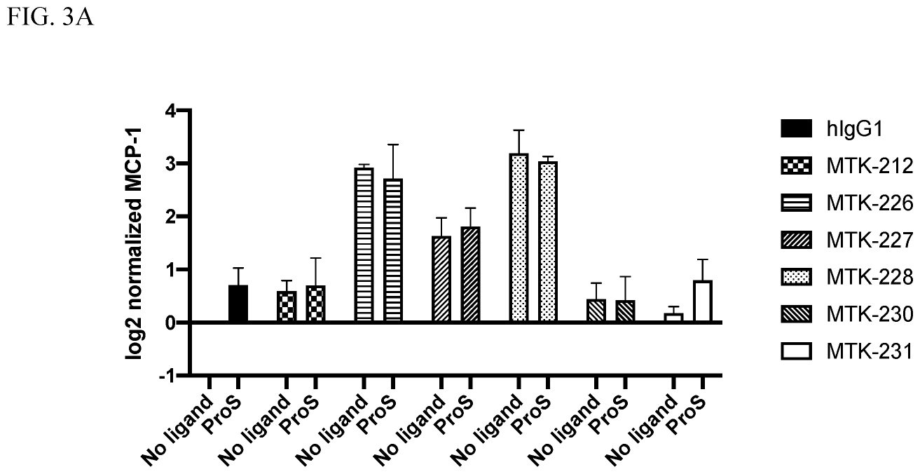

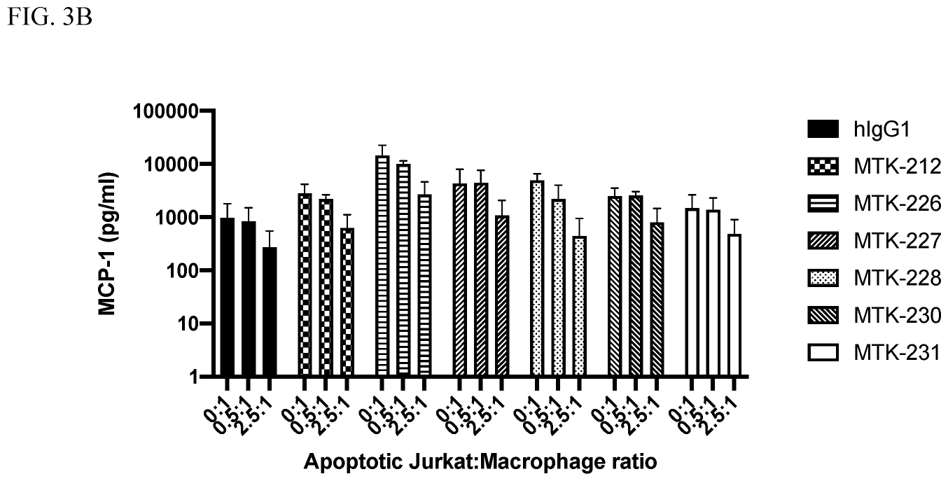

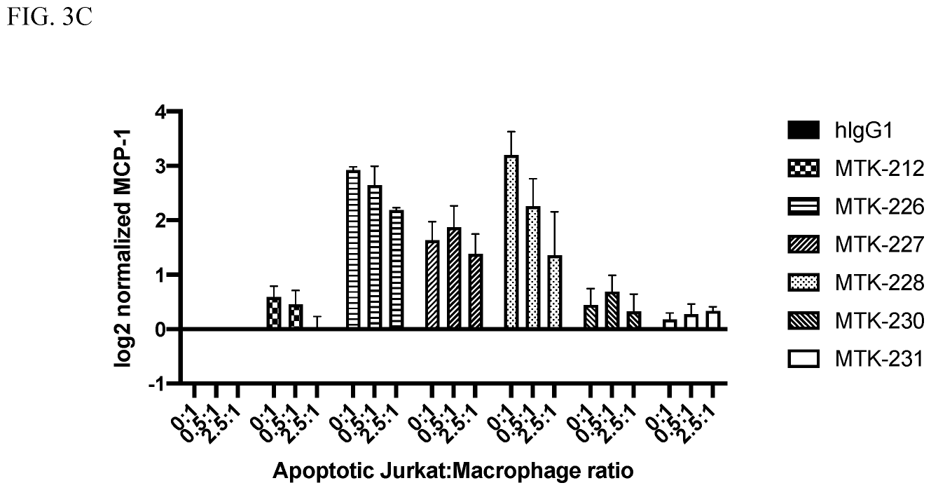

FIG. 3A shows the MCP-1 levels in supernatants, normalized to that observed in cells treated with hIgG1 isotype control antibody in the absence of MerTK ligand ProS. The values in FIG. 3A are plotted on a log 2 scale such that a doubling of the MCP-1 concentration compared to the isotype would have a value of 1, while a halving of the analyte concentration would have a value of −1. The recombinant MerTK ligand ProS alone induced an increase in MCP-1 production. As shown in FIG. 3A, anti-MerTK antibodies of the present disclosure induced an increase in MCP-1 production that was not further enhanced by the addition of the MerTK ligand ProS. In this set of experiments, however, anti-MerTK antibody MTK-231 appeared to require ProS ligand to show an increased MCP-1 levels as measured using this specific assay.

FIG. 3B shows MCP-1 levels measured in the supernatants from these studies. As shown in FIG. 3B, anti-MerTK antibodies of the present disclosure increased MCP-1 levels in the supernatants of macrophages in culture.

FIG. 3C shows the MCP-1 production normalized and plotted on a log 2 scale. As shown in FIG. 3C, addition of increasing amounts of apoptotic Jurkat cells (without ProS) reduced the fold-increase in MCP-1 levels production of MCP-1 associated with anti-MerTK antibodies MTK-226 and MTK-228.

Taken together, these results showing anti-MerTK antibodies of the present disclosure increase MCP-1 levels indicated that anti-MerTK antibodies are effective at agonizing MerTK (i.e., activating or increasing the activity of MerTK) and thus effective at enhancing phagocytosis/efferocytosis.

### Example 18: Effect of Anti-MerTK Antibodies on MerTK Tyrosine Phosphorylation in the Absence or Presence of Gas6 Ligand

The effect of anti-MerTK antibodies on MerTK tyrosine phosphorylation (pMerTK) in myeloid cells in the presence or absence of the MerTK ligand Gas6 was examined as follows. To generate monocyte-derived macrophages, human primary monocytes were isolated from heparinized human blood (Blood Centers of the Pacific) using RosetteSep Human Monocyte Enrichment Cocktail (STEMCELL Technologies), according to the manufacturer's protocol. Monocytes were cultured in RPMI (Invitrogen) containing 10% Fetal Bovine Serum (FBS; Hyclone) and 50 ng/mL M-CSF (Biolegend) to induce differentiation of macrophages. After 6 days, the macrophages were harvested by removing media, incubating with 3 mM EDTA for 5 min at 37° C., and subsequently scraping cells. The macrophages were then plated on 96-well plates at 0.1×10⁶/well and cultured with 50 ng/mL M-CSF (Biolegend), 100 nM Dexamethasone (Tocris), 50 ng/mL human TGFβ (R&D Systems) and 20 ng/mL of IL-10 (Pepro Tech). Two days later (˜48 hours), the cells were serum starved for two hours by removing cell media and replacing with complete growth media minus 10% FBS (serum-free media).

At the end of the two-hour serum starvation period, cell media was removed, and the cells were incubated with various anti-MerTK antibodies in serum-free media in the presence or absence of 200 nM Gas6 protein (R&D Systems). Anti-MerTK antibodies were used at a final concentration of 10 μg/mL and the cells were incubated for 8 min at 37° C. The cells were then harvested by removing treatment media, washed once with ice-cold PBS, and lysed with 150 μL of ice-cold 1× lysis buffer (Cell Signaling Technology). Each 96-well plate was incubated in lysis buffer for 30 min on shaker at 4° C. The plates were then cleared of cellular debris by centrifugation at 4,300×g for 10 min at 4° C. The supernatant was collected for phospho-Mer (panTyr) ELISA (Cell Signaling Technology) and BCA (ThermoFisher Scientific). Lysates were subsequently processed according to manufacturer's instructions and pMerTK levels determined and normalized to total protein measured by BCA (Thermo Fisher Scientific).

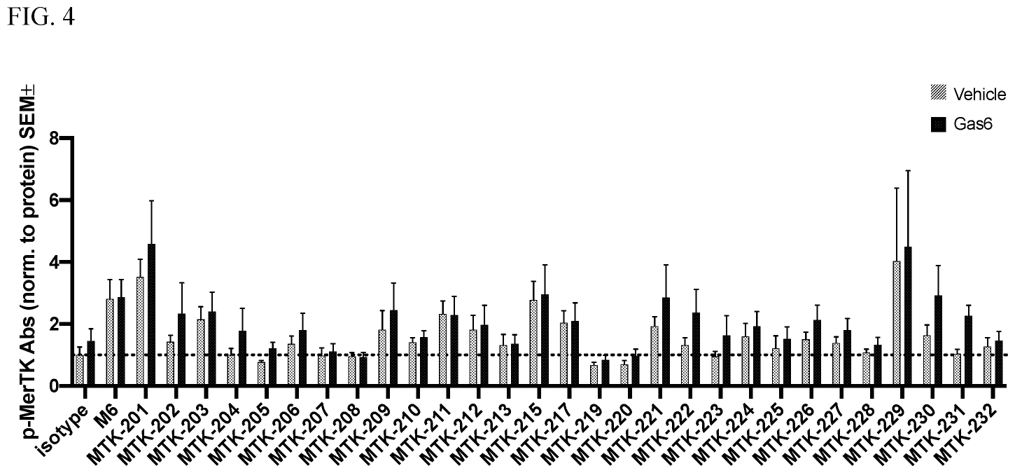

As shown in FIG. 4, several anti-MerTK antibodies of the present disclosure were able to induce pMerTK in the presence (black columns) or absence (grey columns) of Gas6. As expected, Gas6 alone was able to increase pMerTK levels to some degree. Anti-MerTK antibodies MTK-201, MTK-202, MTK-203, MTK-206, MTK-209, MTK-210, MTK-211, MTK-212, MTK-213, MTK-215, MTK-217, MTK-221, MTK-222, MTK-224, MTK-226, MTK-227, MTK-229, MTK-230, and MTK-232 were all effective at increasing pMerTK levels in macrophages on their own, in the absence of Gas6 ligand. For certain anti-MerTK antibodies, addition of Gas6 ligand resulted in a further increase of pMerTK levels above that observed in the absence of Gas6. Anti-MerTK antibodies increased pMerTK levels in macrophages between about 1-fold to about 4-fold above that observed in cells treated with isotype control antibody.

### Example 19: Effect of Anti-MerTK Antibodies on pAKT

The AKT (protein kinase B) signaling pathway is a signal transduction pathway that promotes cell survival and growth. The effect of anti-MerTK antibodies on phosphorylation of AKT (pAKT) was examined as follows. SK-MEL-5 cells from an exponentially growing culture were seeded at a density of 50,000 cells/well on a 96-well plate and incubated overnight. Cells were serum starved for 4 hours. Anti-MerTK antibodies of the present disclosure were added to the cells (66.6 nM final antibody concentration) for 15 minutes at 37° C. The media was then removed from the cells and the cells were lysed for 30 minutes with shaking. pAKT measurements were determined from cell lysates using the Cisbio phosph-AKT (Ser473) Kit (Cisbio, #64AKSPEG) following the manufacturer's instructions for two-plate assay protocol in a 20 mL final volume.

Table 13 below shows fold increase in pAKT levels in SK-MEL-5 cells incubated with anti-MerTK antibodies of the present disclosure over that observed in SK-MEL-5 cells incubated with control IgG antibody. Cells incubated with 200 nM recombinant human Gas6 (R&D Systems) increased pAKT levels by approximately 8-12-fold over that observed in the presence of control IgG antibody.

TABLE 13

| | Fold increase in | |
|---|---|---|
| Antibody | PAKT over IgG control | EC50 (nM) |
| MTK-227 | 4.7 | 1.89 |
| MTK-205 | 4.7 | nd |
| MTK-222 | 4.6 | nd |
| MTK-218 | 4.5 | nd |
| MTK-225 | 4.5 | nd |
| MTK-221 | 4.4 | nd |
| MTK-224 | 4.3 | nd |
| MTK-209 | 4.3 | nd |
| MTK-201 | 4.3 | 0.65 |
| MTK-203 | 4.2 | nd |
| MTK-226 | 4.2 | 0.53 |
| MTK-228 | 4.2 | 0.53 |
| MTK-223 | 4.1 | nd |
| MTK-229 | 4.1 | 1.89 |
| MTK-217 | 4.1 | nd |
| MTK-211 | 4.0 | nd |
| MTK-214 | 4.0 | nd |
| MTK-230 | 3.9 | 2.05 |
| MTK-213 | 3.7 | nd |
| MTK-220 | 3.6 | nd |
| MTK-219 | 3.5 | nd |
| MTK-210 | 3.3 | 14.98 |
| MTK-212 | 3.3 | 96.87 |
| MTK-232 | 3.2 | nd |
| MTK-216 | 3.2 | nd |
| MTK-206 | 3.1 | nd |
| MTK-202 | 3.0 | 1.01 |
| MTK-204 | 3.0 | nd |
| MTK-231 | 3.0 | 0.65 |
| MTK-215 | 2.6 | 208.4 |
| MTK-208 | 2.6 | nd |
| MTK-207 | 1.0 | nd |

nd = not determined

The EC50 values of certain anti-MerTK antibodies of the present disclosure were determined from the pAKT assay described above. The EC50 values are shown above in Table 13.

### Example 20: Domain Binding Analysis of Anti-MerTK Antibodies

The following studies were performed to analyze the binding sites of various anti-MerTK antibodies of the present disclosure on human MerTK.

MerTK is a member of the TAM family, whose members share a unique domain structure containing an N-terminal region (NT), two Immunoglobulin-like domains (Ig1 and Ig2), two Fibronectin type III domains (FN1 and FN2), a juxta-membrane region (JM), and an intracellular tyrosine kinase domain. Cleavage within the juxta-membrane region leads to release of soluble MerTK extracellular domain (ECD). Human MerTK ECD can be divided into the following domains, the amino acid sequences of which are shown below in Table 14:

TABLE 14

| Human MerTK | | SEQ ID |
|---|---|---|
| domain | Amino Acid Sequence | NO: |
| N-terminal domain | AITEAREEAKPYPLFPGPFPGSLQTDHTPLLSLPHAS | 229 |
| (NT) of human MerTK | GYQPALMFSPTQPGRPHTGNVAIPQVTSVE | |
| ECD | | |
| Immunoglobulin-like | SKPLPPLAFKHTVGHIILSEHKGVKFNCSISVPNIYQ | 230 |
| domain (Ig1) of human | DTTISWWKDGKELLGAHHAITQFYPDDEVTAIIASFS | |
| MerTK ECD | ITSVQRSDNGSYICKMKINNEEIVSDPIYIEVQ | |
| Immunoglobulin-like | GLPHFTKQPESMNVTRNTAFNLTCQAVGPPEPVNIFW | 231 |
| domain (Ig2) of human | VQNSSRVNEQPEKSPSVLTVPGLTEMAVFSCEAHNDK | |
| MerTK ECD | GLTVSKGVQIN | |
| Fibronectin type III | IKAIPSPPTEVSIRNSTAHSILISWVPGFDGYSPFRN | 232 |
| domain (FN1) of | CSIQVKEADPLSNGSVMIFNTSALPHLYQIKQLQALA | |
| human MerTK ECD | NYSIGVSCMNEIGWSAVSPWILAST | |
| Fibronectin type III | TEGAPSVAPLNVTVFLNESSDNVDIRWMKPPTKQQDG | 233 |
| domain (FN2) of | ELVGYRISHVWQSAGISKELLEEVGQNGSRARISVQV | |
| human MerTK ECD | HNATCTVRIAAVTRGGVGPFSDPV | |
| Juxta membrane | KIFIPAHGWVDYAPSSTPAPGNADPVLII | 234 |
| domain region (JM) of | | |
| human MerTK ECD | | |

Axl protein, another member of the TAM family, shares a common domain structure to that of MerTK, having the following domains and associated amino acid sequences in its ECD as shown below in Table 15:

TABLE 15

| | | SEQ ID |
|---|---|---|
| Human Axl domain | Amino Acid Sequence | NO: |
| N-terminal domain (NT) | APRGTQAEESPFVGNPGNITGARGLTG | 235 |
| of human Axl ECD | | |
| Immunoglobulin-like | TLRCQLQVQGEPPEVHWLRDGQILELADSTQTQVP | 236 |
| domain (Ig1) of | LGEDEQDDWIVVSQLRITSLQLSDTGQYQCLVFLG | |
| human Axl ECD | HQTFVSQPGYVGLE | |
| Immunoglobulin-like | GLPYFLEEPEDRTVAANTPFNLSCQAQGPPEPVDL | 237 |
| domain (Ig2) of | LWLQDAVPLATAPGHGPQRSLHVPGLNKTSSFSCE | |
| human Axl ECD | AHNAKGVTTSRTATITVLP | |
| Fibronectin type III | QQPRNLHLVSRQPTELEVAWTPGLSGIYPLTHCTL | 238 |
| domain (FN1) of | QAVLSDDGMGIQAGEPDPPEEPLTSQASVPPHQLR | |
| human Axl ECD | LGSLHPHTPYHIRVACTSSQGPSSWTHWLPVETPE | |
| | G | |
| Fibronectin type III | VPLGPPENISATRNGSQAFVHWQEPRAPLQGTLLG | 239 |
| domain (FN2) of | YRLAYQGQDTPEVLMDIGLRQEVTLELQGDGSVSN | |
| human Axl ECD | LTVCVAAYTAAGDGPWS | |
| Juxta membrane domain | LPVPLEAWRPGQAQPVHQLVKEPSTPAFSWPWW | 240 |
| (JM) of human Axl ECD | | |

In these experiments, a series of chimeric proteins were recombinantly expressed in which various domains of human MerTK were swapped with corresponding human Axl domains. The resulting chimeric proteins were recombinantly expressed in Expi293 cells and binding of various anti-MerTK antibodies of the present disclosure were then analyzed for their ability to bind the Axl/MerTK domain-swapped chimeras. DNA encoding the domain-swapped chimeras and deletion mutants, with a signal sequence (MGWSCIILFLVATATGVHS (SEQ ID NO:241) in MerTK constructs, and MGWSCIILFLVATATG (SEQ ID NO:242) in Axl constructs) and a linker+His+Avi tag (GGSGHHHHHHGGGLNDIFEAQKIEWHE; SEQ ID NO:243) were prepared by gene synthesis and cloned into the expression vector pcDNAtopo3.4 (GeneArt, ThermoFisher). Expi293 cells were transfected with 20 μg of plasmids and ExpiFectamine following recommended procedures (ThermoFisher). Transfected cells were grown in 20 mL cultures with shaking at 37° C. and 5% CO2 for four days. Cells were pelleted by centrifugation and the supernatants were filtered through 0.2 μM filters by vacuum.

Table 16 shows the configurations of the various Axl/MerTK domain-swapped chimeras that were generated for use in these studies. In this table an M (MerTK) or an A (Axl) indicates the protein from which that particular domain was included (i.e., swapped) in the corresponding chimeric protein construct; NT (N-terminal domain), Ig1 (immunoglobulin-like domain 1), Ig2 (immunoglobulin-like domain 2), FN1 (fibronectin type III domain 1), FN2 (fibronectin type III domain 2), and JM (juxta-membrane domain).

TABLE 16

| Axl/MerTK Chimeric | NT | Ig1 | Ig2 | FN1 | FN2 | JM |
|---|---|---|---|---|---|---|
| Full MerTK | M | M | M | M | M | M |
| MerTK with swapped NT | A | M | M | M | M | M |
| MerTK with swapped Ig1 | M | A | M | M | M | M |
| MerTK with swapped Ig2 | M | M | A | M | M | M |
| MerTK with swapped FN1 | M | M | M | A | M | M |
| MerTK with swapped FN2 | M | M | M | M | A | M |
| MerTK with swapped JM | M | M | M | M | M | A |
| Full Axl | A | A | A | A | A | A |
| Axl with swapped NT | M | A | A | A | A | A |
| Axl with swapped Ig1 | A | M | A | A | A | A |
| Axl with swapped Ig2 | A | A | M | A | A | A |
| Axl with swapped FN1 | A | A | A | M | A | A |
| Axl with swapped FN2 | A | A | A | A | M | A |
| Axl with swapped JM | A | A | A | A | A | M |
| MerTK with deleted NT | | M | M | M | M | M |
| MerTK with deleted NT/Ig1 | | | M | M | M | M |
| MerTK with deleted | | | | M | M | M |
| NT/Ig1/Ig2 | | | | | | |
| MerTK with deleted | | | | | M | M |
| NT/Ig1/Ig2/FN1 | | | | | | |
| MerTK with deleted | | | | | | M |
| NT/Ig1/Ig2/FN1/FN2 | | | | | | |
| MerTK N-terminal ECD | M | M | M | A | A | A |
| Half-and-half chimera | | | | | | |
| MerTK C-terminal ECD Half- | A | A | A | M | M | M |
| and-half chimera | | | | | | |

Binding of anti-MerTK antibodies of the present disclosure to these domain-swapped chimera or deletion mutants was tested by Surface Plasmon Resonance (SPR) using Carterra LSA. Purified anti-MerTK antibodies were immobilized in duplicate on a HC30M chip (Carterra) by amine coupling, following the manufacturer's instructions (described previously).

Supernatants containing the constructs were diluted 1:1 with running buffer containing 0.5 mg/mL BSA (HBS-TE, Carterra; BSA, Sigma) and injected over the immobilized antibodies. The surface was regenerated with 10 mM Glycine pH2.0 after each construct injection. Sensorgrams were analyzed using Carterra Epitope software to identify patterns of construct binding. Loss of binding to MerTK constructs with domains deleted or swapped in from Axl and/or gain of binding to Axl constructs with MerTK domains swapped in were evaluated. Data from these studies were used to construct a domain binding map for anti-MerTK antibodies and their binding to various domains of human MerTK.

In addition to anti-MerTK antibodies of the present disclosure, the following anti-MerTK antibodies were also used in these studies: mouse anti-human MerTK antibody H1 (BioLegend, Clone ID: 590H11G1E3, mouse IgG1), mouse anti-human MerTK antibody H2 (R&D systems, Clone ID: 125518, mouse IgG2b), mouse anti-human MerTK antibody H3 (R&D systems, Clone ID 125508, mouse IgG2b), mouse anti-human MerTK antibody H6 (eBioscience, Clone ID: A3KCAT, mouse IgG1), mouse anti-human MerTK antibody H7 (Sino Biological, Clone ID: 09, mouse IgG2b), human anti-human MerTK antibody M6 (disclosed in WO2016/106221, huIgG1 LALAPS), human anti-human MerTK antibody CDX Ab2000 (disclosed in WO2019/084307, huIgG1 LALAPS), and human anti-human MerTK antibody CDX Ab3000 (disclosed in WO2020/106461, huIgG1 LALAPS).

The results of these binding studies are shown below in Table 17. The domain(s) required for binding are listed first, and domains identified in parentheses affect binding to a lesser extent, possibly indirectly through conformational effects. Evaluation of binding to the two half-and-half chimera helps to establish and/or confirm the primary binding domain for the antibodies tested here.

TABLE 17

| | huMerTK Domain(s) | |
|---|---|---|
| | required for binding | Half-and-half |
| Antibody | (or affect binding) | Chimera binding |
| H6 | NT | MerTK N-terminal |
| MTK-204 | | ECD |
| MTK-220 | | (NT-Ig1-Ig2) |
| MTK-223 | | + |
| MTK-202 | NT (FN1) | Axl C-terminal ECD |
| MTK-231 | | (FN1-FN2- JM) |
| MTK-213 | Ig1 | |
| MTK-218 | | |
| MTK-225 | | |
| MTK-226 | | |
| MTK-228 | | |
| M6 | | |
| MTK-215 | Ig1 (FN1) | |
| H1 | Ig2, FN1 | Neither half-and- |
| H2 | | half chimera |
| CDX | | |
| AB3000 | | |
| MTK-212 | Ig2, FN1 (FN2) | |
| MTK-203 | FN1 | MerTK C-terminal |
| MTK-209 | | ECD (FN1-FN2-JM) + |
| MTK-214 | | Axl N-terminal ECD |
| MTK-224 | | (NT-Ig1-Ig2) |
| MTK-222 | FN1 (FN2, Ig1) | |
| MTK-227 | | |
| MTK-230 | | |
| MTK-221 | | |
| MTK-206 | FN2, JM (FN1) | |
| MTK-217 | | |
| MTK-229 | | |
| H3 | JM | |
| H7 | | |
| MTK-205 | Fn1, Fn2, JM (Ig1) | |
| MTK-210 | | |
| MTK-219 | | |

As shown in Table 17, anti-MerTK antibodies MTK-204, MTK-220, MTK-223, and H6 bound to chimeric protein constructs containing the N-terminal region of human MerTK. This included the MerTK N-term, but not the MerTK C-term, half-and-half chimera. Anti-MerTK antibodies MTK-202 and MTK-231 also bound to constructs containing the N-terminal region of MerTK, including the MerTK N-term, but not the MerTK C-term half-and-half chimera, but binding was reduced when the MerTK FN1 domain was swapped with the Axl FN1 domain.

Anti-MerTK antibodies MTK-213, MTK-218, MTK-225, MTK-226, and M6 bound to constructs that contained the MerTK Ig1 including the MerTK N-term, but not the MerTK C-term half-and-half chimera. Anti-MerTK antibody MTK-215 bound to chimeric protein constructs that contained the MerTK Ig1, including the MerTK N-term, but not the MerTK C-term half-and-half chimera; however, its binding was reduced when the MerTK FN1 domain was swapped with the Axl FN1 domain.

Antibodies H1, H2, and CDX Ab3000 required the presence of both MerTK Ig2 and Fn1 domains for binding, and did not bind to either half-and-half chimera, suggesting that their binding sites may extend over the junction between these two domains. Similarly, binding by anti-MerTK antibody MTK-212 required the presence of both the Ig2 and FN1 domains (and it also failed to bind either half-and-half chimera), but unlike the above Ig2/FN1 binding antibodies, antibody MTK-212 binding was reduced when the MerTK FN2 domain was swapped with the Axl FN2 domain.

Antibodies MTK-203, MTK-209, MTK-214, and MTK-224 bound to constructs containing the MerTK FN1 domain, including the MerTK C-terminal, but not the MerTK N-terminal half-and-half chimera. Antibodies MTK-222, MTK-227, and MTK-230 also bound constructs containing the MTK FN1 domain, including the MerTK C-term half-and-half, but not the MerTK N-term half-and-half chimera, but their binding was reduced when the MerTK FN2 or Ig1 domains were not present.

Antibodies MTK-206, MTK-217, and MTK-229 bound to constructs containing the MerTK FN2 and JM domains, including the MerTK C-term half-and-half, but not the MerTK N-term half-and-half chimera, while their binding was reduced when the MerTK FN1 domain was not present. Antibodies H3 and H7 bound to constructs that contained the MerTK JM domain as well as the MerTK C-term ECD chimera. Binding of antibodies MTK-205, MTK-210, and MTK-219 was reduced when any of the MerTK C-terminal domains (FN1, FN2, or JM) were not present, and they bound to the MerTK C-term, but not the MerTK N-term half-and-half chimera. Their binding was also reduced in the absence of the MerTK Ig1 domain. None of the anti-MerTK antibodies of the present disclosure bound to the ECD domain of human Axl (data not shown).

In comparing the bin assignments from Table 11 above, with the domain binding from Table 17, anti-MerTK antibodies in epitope bins 1, 2, 3, 4, and 10 include binders to the C-terminal region of the MerTK ECD, while antibodies in epitope bins 6, 7, 8, and 9 include binders to the N-terminal region of the MerTK ECD. Antibodies in epitope bin 5 bind MerTK in a region that requires both the N- and C-terminal regions of the MerTK ECD, potentially spanning the junction between the two regions.

1-4. (canceled)

5. An isolated antibody that binds to human MerTK, wherein the antibody comprises a heavy chain variable region and a light chain variable region, wherein the heavy chain variable region and the light chain variable region comprise:
an HVR-H1, HVR-H2, HVR-H3, HVR-L1, HVR-L2, and HVR-L3 comprising the amino acid sequences of
(i) SEQ ID NOs: 213, 215, 217, 156, 180, and 219, respectively;
(ii) SEQ ID NOs: 64, 82, 111, 138, 164, 186, respectively;
(iii) SEQ ID NOs: 65, 83, 112, 139, 165, 187, respectively;
(iv) SEQ ID NOs: 66, 84, 113, 138, 164, 188, respectively;
(v) SEQ ID NOs: 224, 225, 226, 146, 227, and 228 respectively;
(vi) SEQ ID NOs: 67, 85, 114, 140, 166, and 189, respectively;
(vii) SEQ ID NOs: 68, 86, 115, 141, 167, and 190, respectively;
(viii) SEQ ID NOs: 65, 87, 116, 142, 168, and 191, respectively;
(ix) SEQ ID NOs: 69, 88, 117, 143, 169, and 192, respectively;
(x) SEQ ID NOs: 70, 89, 118, 144, 163, and 193, respectively;
(xi) SEQ ID NOs: 71, 90, 119, 145, 170, 194, respectively;
(xii) SEQ ID NOs: 72, 91, 120, 146, 171, 195, respectively;
(xiii) SEQ ID NOs: 73, 92, 121, 147, 172, 196, respectively;
(xiv) SEQ ID NOs: 65, 93, 122, 148, 173, 197, respectively;
(xv) SEQ ID NOs: 66, 94, 123, 149, 174, 198, respectively;
(xvi) SEQ ID NOs: 66, 95, 124, 150, 164, 188, respectively;
(xvii) SEQ ID NOs: 73, 96, 125, 151, 175, and 199, respectively;
(xviii) SEQ ID NOs: 74, 97, 126, 152, 176, and 200, respectively;
(xix) SEQ ID NOs: 71, 98, 127, 153, 177, and 201, respectively;
(xx) SEQ ID NOs: 66, 99, 128, 138, 164, and 188, respectively;
(xxi) SEQ ID NOs: 75, 100, 129, 154, 178, and 202, respectively;
(xxii) SEQ ID NOs: 71, 101, 130, 155, 179, and 201, respectively;
(xxiii) SEQ ID NOs: 76, 102, 131,155, 179, 201, respectively;
(xxiv) SEQ ID NOs: 77, 103, 132, 157, 181, and 204, respectively;
(xxv) SEQ ID NOs: 78, 104, 133, 158, 182, and 205, respectively;
(xxvi) SEQ ID NOs: 74, 105, 126, 159, 176, and 200, respectively;
(xxvii) SEQ ID NOs: 79, 106, 134, 160, 183, and 206, respectively;
(xxviii) SEQ ID NOs: 74, 107, 126, 161, 176, and 200, respectively;
(xxix) SEQ ID NOs: 70, 108, 135, 144, 170, and 207, respectively;
(xxx) SEQ ID NOs: 80, 109, 136, 162, 184, and 208, respectively;
(xxxi) SEQ ID NOs: 63, 81, 110, 137, 163, and 185, respectively; or
(xxxii) SEQ ID NOs: 214, 216, 218, 220, 172, and 221, respectively.

6-11. (canceled)

12. The antibody of claim 5, wherein the antibody comprises a heavy chain variable region and a light chain variable region comprising the amino acid sequences of SEQ ID NOs:209 and 211, respectively; SEQ ID NOs:5 and 34, respectively; SEQ ID NOs:6 and 35, respectively; SEQ ID NOs:7 and 36, respectively; respectively; SEQ ID NOs:8 and 37, respectively; SEQ ID NOs:222 and 223, respectively; SEQ ID NOs:9 and 38, respectively; SEQ ID NOs:10 and 39, respectively; SEQ ID NOs:11 and 40, respectively; SEQ ID NOs:12 and 41, respectively; SEQ ID NOs:13 and 42, respectively; SEQ ID NOs:14 and 43, respectively; SEQ ID NOs:15 and 44, respectively; SEQ ID NOs:16 and 45, respectively; SEQ ID NOs:17 and 46, respectively; SEQ ID NOs:18 and 47, respectively; SEQ ID NOs:19 and 48, respectively; SEQ ID NOs:20 and 49, respectively; SEQ ID NOs:21 and 50, respectively; SEQ ID NOs:22 and 51, respectively; SEQ ID NOs:23 and 52, respectively; SEQ ID NOs:24 and 53, respectively; SEQ ID NOs:25 and 54, respectively; SEQ ID NOs:26 and 55, respectively; SEQ ID NOs:27 and 56, respectively; SEQ ID NOs:28 and 57, respectively; SEQ ID NOs:29 and 58, respectively; SEQ ID NOs:30 and 59, respectively; SEQ ID NOs:31 and 60, respectively; SEQ ID NOs:32 and 61, respectively; SEQ ID NOs:33 and 62, respectively; and SEQ ID NOs:210 and 212, respectively.

13. An isolated antibody that binds to human MerTK, wherein the antibody competitively inhibits binding of the antibody of claim 5 for binding to human MerTK.

14. An isolated antibody that binds to human MerTK, wherein the antibody binds, the same, essentially the same, or an overlapping epitope on MerTK as the antibody of claim 5.

15. The antibody of claim 5, wherein the antibody does not reduce efferocytosis by more than 40%.

16-20. (canceled)

21. The antibody of claim 5, wherein the antibody increases phagocytosis by a phagocytic cell.

22-23. (canceled)

24. The antibody of claim 5, wherein the antibody does not reduce binding of Gas6 or ProS to MerTK by more than 30%, by more than 20%, by more than 10%, or by more than 5%.

25. The antibody of claim 5, wherein the antibody increases phosphorylation of MerTK.

26-28. (canceled)

29. The antibody of claim 5, wherein the antibody increases monocyte chemoattractant protein-1 (MCP-1) expression in macrophages.

30-31. (canceled)

32. The antibody of claim 5, wherein the antibody binds to the N-terminal domain of MerTK, Ig-like domain 1, Ig-like domain 2, fibronectin type III domain 1, fibronectin type III domain 2, and/or juxtamembrane domain of MerTK.

33. The antibody of claim 5, wherein the antibody binds to cynomolgus MerTK, but not murine MerTK, binds to murine MerTK, but not cynomolgus MerTK, or binds to cynomolgus and murine MerTK.

34. The antibody of claim 5, wherein the antibody binds human MerTK with an affinity of less than 440 nM, less than 400 nM, less than 350 nM, less than 300 nM, less than 250 nM, less than 200 nM, less than 150 nM, less than 100 nM, or less than 50 nM.

35-36. (canceled)

37. The antibody of claim 5, wherein the antibody is a murine antibody, a human antibody, a humanized antibody, a bispecific antibody, a monoclonal antibody, a multivalent antibody, a conjugated antibody, or a chimeric antibody.

38. The antibody of claim 5, wherein the antibody is of the IgG class, the IgM class, or the IgA class.

39. The antibody of claim 38, wherein the antibody is of the IgG class and has an IgG1, an IgG2, or an IgG4 isotype.

40. The antibody of claim 5, wherein the antibody is a full-length antibody.

41. The antibody of claim 5, wherein the antibody is an antibody fragment.

42. The antibody of claim 41, wherein the fragment is a Fab, Fab′, Fab′-SH, F(ab′)2, Fv or scFv fragment.

43. An isolated nucleic acid comprising a nucleic acid sequence encoding the antibody of claim 5.

44. A vector comprising the nucleic acid of claim 43.

45. An isolated host cell comprising the nucleic acid of claim 43.

46. (canceled)

47. A method of producing an antibody that binds to human MerTK, the method comprising culturing the cell of claim 45 so that the antibody is produced.

48-49. (canceled)

50. A pharmaceutical composition comprising the antibody of claim 5 and a pharmaceutically acceptable carrier.

51. A method of preventing, reducing risk, or treating an autoimmune disorder in an individual, the method comprising administering to an individual in need thereof a therapeutically effective amount of the antibody of claim 5.

52. (canceled)

53. A method of preventing, reducing risk, or treating a retinal ganglion degenerative disorder or retinitis pigmentosa in an individual, the method comprising administering to an individual in need thereof a therapeutically effective amount of the antibody of claim 5.

54. A method of preventing, reducing risk, or treating vision loss in an individual, the method comprising administering to an individual in need thereof a therapeutically effective amount of the antibody of claim 5.

55. A method for detecting MerTk in a sample comprising contacting said sample with the antibody of claim 5.

56. A method of increasing phagocytosis comprising contacting a phagocytic cell with the antibody of claim 5.

57-60. (canceled)
